(19) United States
(12) Patent Application Publication
(10) Pub. No.: US 2004/0221327 A1
(43) Pub. Date: Nov. 4, 2004

(54) Antibodies against autoantigens of primary billary cirrhosis and methods of making and using them

(76) Inventor: M. Eric Gershwin, Davis, CA (US)

Correspondence Address: Jane T Gunnison, Fish & Neave, 1251 Avenue of the Americas, New York, NY, 10020-1104, US

(21) Appl. No.: 10/473,287

(22) PCT Filed: Mar. 27, 2002
(86) PCT No.: PCT/US02/09694

(30) Foreign Application Priority Data
Mar. 27, 2001 (US) 60279052
Sep. 21, 2001 (US) 60323920

(51) Int. Cl. C12P 21/04; A01K 67/027

(52) U.S. Cl. 800/6; 435/70.21

(57) ABSTRACT

The present invention provides a method of making an isolated human monoclonal antibody, said antibody binds a mitochondrial antigen bound by a human autoantibody found in patients with PBC. Said method involves using a transgenic non-human animal that has the ability to make human antibodies.

The present invention also relates to a human monoclonal antibody that binds a mitochondrial antigen bound by a human autoantibody found in patients with PBC as well as a cell line making this antibody.

The present invention also relates to a transgenic non-human animal that has the ability to make human antibodies, wherein said animal makes a human monoclonal antibody that specifically binds a mitochondrial antigen bound by a human autoantibody found in patients with primary biliary cirrhosis. This invention also relates to methods of identifying antagonists of the antibodies that bind a mitochondrial antigen bound by a human autoantibody found in patients with PBC.

The present invention also provides an isolated protein bound by human monoclonal antibody 3E7, wherein said isolated protein is a mitochondrial protein and has a molecular weight of approximately 100 kilodaltons as determined by SDS-PAGE.

This application claims benefit under 35 U.S.C. § 119(e) of U.S. provisional application No. 60/279,052, filed Mar. 27, 2001 and U.S. provisional application No. 60/323,920, filed Sep. 21, 2001. The disclosures of U.S. provisional applications Nos. 60/279,052 and 60/323,920 are incorporated by reference herein.

This invention was made in part with United States government support under grant number NIH DK39588 awarded by the National Institutes of Health. The United States government may have certain rights in the invention.

### TECHNICAL FIELD OF THE INVENTION

The present invention relates to the field of autoimmune diseases. The specific autoimmune disease is primary biliary cirrhosis.

### BACKGROUND OF THE INVENTION

Anti-mitochondrial antibodies are detected in greater than 95% of sera from patients with primary biliary cirrhosis (“PBC”). These autoantibodies recognize the highly conserved mitochondrial 2-oxo-acid dehydrogenase complex (OADC enzyme complex), including the E2 component (subunit) of the pyruvate dehydrogenase complex (“PDC-E2”), the E2 component (subunit) of the branched-chain 2-oxo-acid dehydrogenase complex (“BCOADC-E2”), the E2 component (subunit) of the 2-oxoglutarate dehydrogenase complex (“OGDC-E2”) and E3-binding protein (“E3BP”)(or protein X). See Van de Water J et al., Journal of Immunology 141:2321-2324 (1988); Gershwin M. E. et al., Immunol Rev 174: 210-225 (2000); Fussey S P et al., Proc. Natl. Acad. of Sci. USA 85:8654-8658 (1988); Fregeau D R et al., Hepatology 11:975-981 (1990) and Surh C D et al., Hepatology 10:127-133 (1989).

Analysis of gene usage of PDC-E2-specific human monoclonal autoantibody immunoglobulin genes showed that sequences of the V_{H} region were restricted, in a way consistent with a model of antigen-driven clonal selection.

Thus, utilization of the specific members of the human immunoglobulin (Ig) gene repertoire may play a critical role in specific epitope recognition of the antigen PDC-E2, resulting in the initiation of a cascade of mucosal autoimmune response against the mitochondrial autoantigens. This may be due either to specific chemical features of the autoantigens or to specific oddities of the etiologic process.

The Ig gene usage of murine monoclonal antibodies directed against these human mitochondrial antigens have not duplicated the human Ig gene repertoire of the human monoclonal autoantibodies. Human monoclonal antibodies that can duplicate the gene usage of autoantibodies from patients with PBC do not exist and are needed.

### SUMMARY OF THE INVENTION

The present invention relates to an isolated antibody that binds one or more mitochondrial antigens bound by a human autoantibody found in patients with PBC. The antibody is preferably produced by methods of this invention. The present invention relates to the heavy chain of said antibody, or the variable region thereof, or portions thereof (including, without limitation, the CDR1-CDR3 and the individual CDRS), wherein said variable region may or may not have the leader sequence attached. The present invention also relates to antibodies that are anti-idiotype antibodies of the isolated antibody that binds one or more mitochondrial antigens bound by a human autoantibody found in patients with PBC.

The present invention provides a method of making an isolated human monoclonal antibody, said antibody binds a mitochondrial antigen bound by a human autoantibody found in patients with PBC. The present invention also relates to a method of obtaining antagonist of a human antibody that specifically binds a mitochondrial antigen bound by a human autoantibody found in patients with primary biliary cirrhosis. Said methods involve using a transgenic non-human animal that has the ability to make human antibodies and that is preferably deficient in the ability to make said animal's antibodies.

The present invention also relates to an immortalized hybridoma cell line making the antibodies of this invention.

The present invention also relates to a transgenic non-human animal that has the ability to make human antibodies and that preferably is deficient in the ability to make said animal's antibodies, wherein said animal makes a human antibody that specifically binds a mitochondrial antigen bound by a human autoantibody found in patients with primary biliary cirrhosis.

The present invention also relates to methods of identifying an antagonist of an antibody that binds a mitochondrial antigen bound by a human autoantibody found in patients with PBC.

The present invention also provides a method of treating or preventing primary biliary cirrhosis, comprising the step of modifying or blocking the epitope on some or all mitochondrial antigens for an antibody that binds to mitochondrial antigens bound by a human autoantibody found in patients with primary biliary cirrhosis.

The present invention also provides an isolated protein bound by human monoclonal antibody 3E7, wherein said isolated protein is a mitochondrial protein (could be, for example, human) and has a molecular weight of approximately 100 kilodaltons as determined by SDS-PAGE.

The foregoing and other objects, features and advantages of the present invention, as well as the invention itself, will be more fully understood from the following description of preferred embodiments.

### BRIEF DESCRIPTION OF THE DRAWINGS

FIG. 1.

Immunoreactivity of human mAbs obtained from XENOMOUSE® animals by immunoblot against beef heart mitochondria (BHM). Lanes: 1, 2C8; 2, 2E6; 3,3D8; 4,4B10; 5, PBC sera (positive control); 6, control human sera. Human mabs 2E6 and 2C8 obtained from XENOMOUSE® animals recognized only PDC-E2 (74 kDa), while 3D8 and 4B10 recognized both PDC-E2 and OGDC (48 kDa).

FIGS. 2A, 2B and 2C.

Hep-2: Classic anti-mitochondrial antibody.

Reactivity of human mabs obtained from XENOMOUSE® animals on Hep-2 cell.

FIGS. 2A, 2B, 2C. Human mAbs (such as human mAb 2E6) obtained from XENOMOUSE® animals showed cytoplasmic speckled pattern corresponding to mitochondrial staining. FIG. 2A: Human mAb 2E6 (Human Mab2E6=human mAb 2E6); FIG. 2B: PBC sera; FIG. 2C: anti-KLH (negative control).

FIGS. 3A, 3B, 3C and 3D. Immunohistochemical staining using human mAbs obtained from XENOMOUSE® animals.

FIG. 3A. Apical staining of septal bile ducts by human mAb 2E6. PBC liver.

Apical regions of a septal bile duct PBC liver (arrow) is positive for Human mAb 2E6 against PDC-E2.

FIG. 3B. 2E6 staining small bile duct cells (PBC). Bile ductules and scattered periportal hepatocytes of PBC liver are also positively stained by human mAb 2E6 in their cytoplasms.

FIG. 3C. Normal liver by 2E6.

Apical regions of a septal bile duct in normal liver(arrow) is positively stained by human mAb 2E6.

FIG. 3D. 3E7 control liver.

Apical region of septal bile of primary sclerosing cholangitis (“PSC”) liver is positively stained by human mAb 3E7 against 100 kD protein of mitochondria.

FIG. 4.

Amino Acid Sequence Comparison of the anti-mitochondrial antigen human mAbs 1F8, 4B10 and 3D8 obtained from XENOMOUSE® animals. Ig H chain.

Comparisons are made to the closest germ line gene. indicates identity to the corresponding amino acid residue in the top line (V_{H}3-21).

Amino acid sequence of the VDJ region of the heavy chain of human mAb 1F8 (SEQ ID NO: 1):

GGLVKPGGSLRLSCAASGFT[FSSYSMN]WVRQAPGKGLEWVS(SISSSSSYIYY ADSVKG)RFTISRDNAKNSLYLQMNSLRAEDTAVYYCAS{SGYSSSWSDY}WGQG TLVTVSS

The sequence within [ ] is CDR1. The sequence within ( ) is CDR2. The sequence within { } is CDR3.

Amino acid sequence of the VDJ region of the heavy chain of human mAb 4B10 (SEQ ID NO: 2):

GGLVKPGGSLRLSCAASGFT[FSSYSMN]WVRQAPGKGLEWVS(SISSSSSYIYY ADSVKG)RFTISRDNAKNSLYLQMNSLRAEDTAVYYCAS{SGYSSSWSDY}WGQG TLVTVSS

The sequence within [ ] is CDR1. The sequence within ( ) is CDR2. The sequence within { } is CDR3.

Amino acid sequence of the VDJ region of the heavy chain of human mAb 3D8 (SEQ ID NO: 3):

GGLVKPGGSLRLSEVSGFT[FSSYSMN]WVRQAPGKGLEWVS(SISSSSSYIYYA DSVKG)RFTISRDNAKNSLYLQMNSLRAEDTAVYYCAI{SVVDY}WGQGTLVTVSS

The sequence within [ ] is CDR1. The sequence within ( ) is CDR2. The sequence within { } is CDR3.

Amino acid sequence of the VDJ region of V_{H}3-21 (SEQ ID NO: 4):

GGLVKPGGSLRLSCAASGFT[FSSYSMN]WVRQAPGKGLEWVS(SISSSSSYIYY ADSVKG)RFTISRDNAKNSLYLQMNSLRAEDTAVYYCAR

The sequence within [ ] is CDR1. The sequence within ( ) is CDR2.

FIG. 5.

Amino Acid Sequence Comparison of the anti-mitochondrial antigen human mAbs 3B4, 4G6, 1F8, 4B10 and 3D8 obtained from XENOMOUSE® animals. Ig L chain. Comparisons are made to the closest germ line gene. For comparison of V,—indicates identity to the corresponding germline amino acid residue in the top line (V_{K}1-17). For comparison of J,—indicates identity to the corresponding amino acid residue in the top line (J_{K}1 of 3B4) for 4G6 and 1F8;—indicates identity to the corresponding amino acid residue in 1F8 (J_{K}3) (third line from the top) for 4B10 and 3D8.

Amino acid sequence of the VJ region of the light chain of human mAb 3B4 (SEQ ID NO: 6):

LSASVGDRVTITC[RASQGIRNDLG]WYQQKPGKAPKRLIY(AASSLQS)GVPSR FSGSGSGTEFTLTISSLQPEDFATYYC{LQHNSYP}RTFGQGTKVEIK

The sequence within [ ] is CDR1. The sequence within ( ) is CDR2. The sequence within { } is CDR3.

Amino acid sequence of the VJ region of the light chain of human mAb 4G6 (SEQ ID NO: 7):

LSASVGDRVTITC[RASQGIRNNLG]WYQQKPGKAPKRLIY(AASSLQS)GVPSR FSGSGSGTEFTLTISSLQPEDFATYYC{LQHNSYP}CSFGQGTKLEIK

The sequence within [ ] is CDR1. The sequence within ( ) is CDR2. The sequence within { } is CDR3.

Amino acid sequence of the VJ region of the light chain of human mAb 1F8 (SEQ ID NO: 8):

LSASVGDRVTITC[RASQGIRNDLG]WYQQKPGKAPKRLIY(AASSLQS)GVPSR FSGSGSGTEFTLTISSLQPEDFATYYC{LQYNSYP}FTFGPGTKVDIK

The sequence within [ ] is CDR1. The sequence within ( ) is CDR2. The sequence within { } is CDR3.

Amino acid sequence of the VJ region of the light chain of human mAb 4B10 (SEQ ID NO: 9):

LSASVGDRVTITC[RASQGIRNDLG]WYQQKPGKAPKRLIY(AASSLQS)GVPSR FSGSGSGTEFTLTISSLQPEDFATYYC{LQYNSYP}FTFGPGTKVDIK

The sequence within [ ] is CDR1. The sequence within ( ) is CDR2. The sequence within { } is CDR3.

Amino acid sequence of the VJ region of the light chain of human mAb 3D8 (SEQ ID NO: 10):

LSASVGDRVTITC[RASQGIRNDLG]WYQQKPGKAPKRLIY(AASSLQS)GVPSR FSGSGSGTEFTLTISSLQPEDFATYYC{LQYNSYP}FIFGPGTKVDIK

The sequence within [ ] is CDR1. The sequence within ( ) is CDR2. The sequence within { } is CDR3.

Amino acid sequence of the VJ region of V_{K}1-17 (SEQ ID NO: 11):

LSASVGDRVTITC[RASQGIRNDLG]WYQQKPGKAPKRLIY(AASSLQS)GVPSR FSGSGSGTEFTLTISSLQPEDFATYYC{LQHNSYP}

The sequence within [ ] is CDR1. The sequence within ( ) is CDR2.

FIG. 6 is a table showing characterization of human mAbs, obtained from XENOMOUSE® animals, to mitochondrial proteins.

PDC=PDC-E2; P, ILD=inner lipoyl domain of PDC-E2; OGDC=OGDC-E2; BCOADC=BCOADC-E2; MIT-3=MIT3.

FIG. 7 tabulates results of immunohistochemistry using human monoclonal antibodies.

Grading (0-3); 0, negative; 1, weakly positive; 2, moderately positive; 3, intensely positive BD, bile duct; ILBD, Bd, interlobular bile duct and bile ductules, a, apical staining;

*, positive in scattered cells

FIG. 8 is a table showing Ig V gene elements used by Human mAbs, obtained from XENOMOUSE® animals, to mitochondrial proteins.

FIG. 9 Cf.Ig V gene elements used by previously-reported Abs.

FIG. 9 is a table showing Ig V gene elements used by previously-reported Abs.

Pascual, 94 (see, e.g., Pascual V et al., J. Immunol. 152: 2577-2585 (1994)). Thomson, 98 (see, e.g., Thomson R K et al., J. Hepatology 28:582-594 (1998)). Fukushima, 20 (see, e.g., Fukushima N et al., Int. Immunol. 7:1047-1055 (1995) and Fukushima N et al., J Autoimmune 14:247-257 (2000)).

### DETAILED DESCRIPTION OF THE INVENTION

### Definitions and General Techniques

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics, virology and protein and nucleic acid chemistry and hybridization described herein are those well known and commonly used in the art. The methods and techniques of the present invention are generally performed according to conventional methods well known in the art and as described in various general and more specific references that are cited and discussed throughout the present specification unless otherwise indicated. See, e.g., Sambrook et al. Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992), and Harlow and Lane Antibodies: A Laboratory Manual, Cold Spring Harbor Laboratory Press, Cold Spring Harbor, N.Y. (1990), which are incorporated herein by reference. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

AMINO ACID ABBREVIATIONS

| | | |
|---|---|---|
| A = | Ala = | Alanine |
| V = | Val = | Valine |
| L = | Leu = | Leucine |
| I = | Ile = | Isoleucine |
| P = | Pro = | Proline |
| F = | Phe = | Phenylalanine |
| W = | Trp = | Tryptophan |
| M = | Met = | Methionine |
| G = | Gly = | Glycine |
| S = | Ser = | Serine |
| T = | Thr = | Threonine |
| C = | Cys = | Cysteine |
| Y = | Tyr = | Tyrosine |
| N = | Asn = | Asparagine |
| Q = | Gln = | Glutamine |
| D = | Asp = | Aspartic Acid |
| E = | Glu = | Glutamic Acid |
| K = | Lys = | Lysine |
| R = | Arg = | Arginine |
| H = | His = | Histidine |

The following terms, unless otherwise indicated, shall be understood to have the following meanings:

The term “isolated protein” or “isolated polypeptide” is a protein or polypeptide that by virtue of its origin or source of derivation (1) is not associated with naturally associated components that accompany it in its native state, (2) is free of other proteins from the same species (3) is expressed by a cell from a different species, or (4) does not occur in nature. Thus, a polypeptide that is chemically synthesized or synthesized in a cellular system different from the cell from which it naturally originates will be “isolated” from its naturally associated components. A protein may also be rendered substantially free of naturally associated components by isolation, using protein purification techniques well known in the art.

A protein or polypeptide is “substantially pure,”“substantially homogeneous” or “substantially purified” when at least about 60 to 75% of a sample exhibits a single species of polypeptide. The polypeptide or protein may be monomeric or multimeric. A substantially pure polypeptide or protein will typically comprise about 50%, 60, 70%, 80% or 90% W/W of a protein sample, more usually about 95%, and preferably will be over 99% pure. Protein purity or homogeneity may be indicated by a number of means well known in the art, such as polyacrylamide gel electrophoresis of a protein sample, followed by visualizing a single polypeptide band upon staining the gel with a stain well known in the art. For certain purposes, higher resolution may be provided by using HPLC or other means well known in the art for purification.

An “immunoglobulin” is a tetrameric molecule. In a naturally-occurring immunoglobulin, each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as κ and λ light chains. Heavy chain constant regions are classified as μ, Δ, γ, α, or ε, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. (1989) and 3rd ed. (1999) Raven Press, N.Y. (incorporated by reference in their entirety for all purposes). The variable regions of each light/heavy chain pair form the antibody binding site such that an intact immunoglobulin generally has at least two binding sites.

Immunoglobulin chains exhibit the same general structure of relatively conserved framework regions (FR) joined by three hypervariable regions, also called complementarity determining regions or CDRs. The CDRs from the two chains of each pair are aligned by the framework regions, enabling binding to a specific epitope. From N-terminus to C-terminus, both light and heavy chains comprise the domains FR1, CDR1, FR2, CDR2, FR3, CDR3 and FR4. The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987); Chothia et al. Nature 342:878-883 (1989).

An “antibody” refers to an intact immunoglobulin, or to an antigen-binding portion thereof that competes with the intact antibody for specific binding. Antigen-binding portions may be produced by recombinant DNA techniques or by enzymatic or chemical cleavage of intact antibodies. Antigen-binding portions include, inter alia, Fab, Fab′, F(ab′)₂, Fv, dAb, and complementarity determining region (CDR) fragments, single-chain antibodies (scFv), chimeric antibodies, diabodies and polypeptides that contain at least a portion of an immunoglobulin that is sufficient to confer specific antigen binding to the polypeptide. An Fab fragment is a monovalent fragment consisting of the VL, VH, CL and CH1 domains; a F(ab′)₂ fragment is a bivalent fragment comprising two Fab fragments linked by a disulfide bridge at the hinge region; a Fd fragment consists of the VH and CH1 domains; an Fv fragment consists of the VL and VH domains of a single arm of an antibody; and a dAb fragment (Ward et al., Nature 341:544-546, 1989) consists of a VH domain. A single-chain antibody (scFv) is an antibody in which a VL and VH regions are paired to form a monovalent molecules via a synthetic linker that enables them to be made as a single protein chain (Bird et al., Science 242:423-426, 1988 and Huston et al., Proc. Natl. Acad. Sci. USA 85:5879-5883, 1988). Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al., Proc. Natl. Acad. Sci. USA 90:6444-6448, 1993, and Poljak, R. J., et al., Structure 2:1121-1123, 1994). One or more CDRs may be incorporated into a molecule either covalently or noncovalently to make it an immunoadhesin. An immunoadhesin may incorporate the CDR(s) as part of a larger polypeptide chain, may covalently link the CDR(s) to another polypeptide chain, or may incorporate the CDR(s) noncovalently. The CDRs permit the immunoadhesin to specifically bind to a particular antigen of interest.

An antibody may have one or more binding sites. If there is more than one binding site, the binding sites may be identical to one another or may be different. For instance, a naturally-occurring immunoglobulin has two identical binding sites, a single-chain antibody or Fab fragment has one binding site, while a “bispecific” or “bifunctional” antibody has two different binding sites.

An “isolated antibody” is an antibody that (1) is not associated with naturally-associated components, including other naturally-associated antibodies, that accompany it in its native state, (2) is free of other proteins from the same species, (3) is expressed by a cell from a different species, or (4) does not occur in nature.

The term “human antibody” includes all antibodies that have one or more variable and constant regions derived from human immunoglobulin sequences. These antibodies may be prepared in a variety of ways, as described below.

A humanized antibody is an antibody that is derived from a non-human species, in which certain amino acids in the framework and constant domains of the heavy and light chains have been mutated so as to avoid or abrogate an immune response in humans. Alternatively, a humanized antibody may be produced by fusing the constant domains from a human antibody to the variable domains of a non-human species. Examples of how to make humanized antibodies may be found in U.S. Pat. Nos. 6,054,297, 5,886,152 and 5,877,293.

The term “chimeric antibody” refers to an antibody that contains one or more regions from one antibody and one or more regions from one or more other antibodies. For example, one or more of the CDRs are derived from a human antibody that binds to a mitochondrial antigen bound by an autoantibody found in patients with PBC. Alternatively, all of the CDRs are derived from a human antibody that binds a mitochondrial antigen bound by an autoantibody from patients with PBC. Alternatively, the CDRs from more than one human antibodies that binds a mitochondrial antigen bound by an autoantibody from patients with PBC, are mixed and matched in a chimeric antibody. For instance, a chimeric antibody may comprise a CDR1 from the light chain of a first human antibody that binds a mitochondrial antigen bound by an autoantibody from patients with PBC may be combined with CDR2 and CDR3 from the light chain of a second human antibody that binds a mitochondrial antigen bound by an autoantibody from patients with PBC, and the CDRs from the heavy chain may be derived from a third human antibody that binds a mitochondrial antigen bound by an autoantibody from patients with PBC. Further, the framework regions may be derived from one of the same antibodies, from one or more different human antibodies, or from a humanized antibody.

The term “surface plasmon resonance”, as used herein, refers to an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.). For further descriptions, see Jonsson, U., et al. (1993) Ann. Biol. Clin. 51:19-26; Jonsson, U., et al. (1991) Biotechniques 11:620-627; Johnsson, B., et al. (1995) J. Mol. Recognit. 8:125-131; and Johnnson, B., et al. (1991) Anal. Biochem. 198:268-277.

The term “K_{off}” refers to the off rate constant for dissociation of an antibody from the antibody/antigen complex.

The term “Kd ” refers to the dissociation constant of a particular antibody-antigen interaction.

Fragments or analogs of antibodies or immunoglobulin molecules can be readily prepared by those of ordinary skill in the art following the teachings of this specification. Preferred amino- and carboxy-termini fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. Bowie et al. Science 253:164 (1991).

Preferred amino acid substitutions are those which: (1) reduce susceptibility to proteolysis, (2) reduce susceptibility to oxidation, (3) alter binding affinity for forming protein complexes, (4) alter binding affinities, and (4) confer or modify other physicochemical or functional properties of such analogs. Analogs can include various muteins of a sequence other than the naturally-occurring peptide sequence. For example, single or multiple amino acid substitutions (preferably conservative amino acid substitutions) may be made in the naturally-occurring sequence (preferably in the portion of the polypeptide outside the domain(s) forming intermolecular contacts). A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et at. Nature 354:105 (1991), which are each incorporated herein by reference.

As used herein, the twenty conventional amino acids and their abbreviations follow conventional usage. See Immunology—A Synthesis (2^{nd} Edition, E. S. Golub and D. R. Gren, Eds., Sinauer Associates, Sunderland, Mass. (1991)), which is incorporated herein by reference. Stereoisomers (e.g., D-amino acids) of the twenty conventional amino acids, unnatural amino acids such as α-, α-disubstituted amino acids, N-alkyl amino acids, lactic acid, and other unconventional amino acids may also be suitable components for polypeptides described herein. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, s-N-methylarginine, and other similar amino acids and amino acids (e.g., 4-hydroxyproline). In the polypeptide notation used herein, the lefthand direction is the amino terminal direction and the right-hand direction is the carboxy-terminal direction, in accordance with standard usage and convention.

The term “polynucleotide” as referred to herein means a polymeric form of nucleotides of at least 10 bases in length, either ribonucleotides or deoxynucleotides or a modified form of either type of nucleotide. The term includes single and double stranded forms of DNA.

The term “isolated polynucleotide” as used herein shall mean a polynucleotide of genomic, cDNA, or synthetic origin or some combination thereof, which by virtue of its origin the “isolated polynucleotide” (1) is not associated with all or a portion of a polynucleotide in which the “isolated polynucleotide” is found in nature, (2) is operably linked to a polynucleotide which it is not linked to in nature, or (3) does not occur in nature as part of a larger sequence.

The term “oligonucleotide” referred to herein includes naturally occurring, and modified nucleotides, linked together by naturally occurring, and non-naturally occurring oligonucleotide linkages. oligonucleotides are a polynucleotide subset generally comprising a length of 200 bases or fewer. Preferably oligonucleotides are 10 to 60 bases in length and most preferably 12, 13, 14, 15, 16, 17, 18, 19, or 20 to 40 bases in length. Oligonucleotides are usually single stranded, e.g., for probes; although oligonucleotides may be double stranded, e.g., for use in the construction of a gene mutant. Oligonucleotides can be either sense or antisense oligonucleotides.

The term “naturally occurring nucleotides” referred to herein includes deoxyribonucleotides and ribonucleotides. The term “modified nucleotides” referred to herein includes nucleotides with modified or substituted sugar groups and the like. The term “oligonucleotide linkages” referred to herein includes oligonucleotides linkages such as phosphorothioate, phosphorodithioate, phosphoroselenoate, phosphorodiselenoate, phosphoroanilothioate, phoshoraniladate, phosphoroamidate, and the like. See e.g., LaPlanche et al. Nucl. Acids Res. 14:9081 (1986); Stec et al. J. Am. Chem. Soc. 106:6077 (1984); Stein et al. Nucl. Acids Res. 16:3209 (1988); Zon et al. Anti-Cancer Drug Design 6:539 (1991); Zon et al. Oligonucleotides and Analogues: A Practical Approach, pp. 87-108 (F. Eckstein, Ed., Oxford University Press, Oxford England (1991)); Stec et al. U.S. Pat. No. 5,151,510; Uhlmann and Peyman Chemical Reviews 90:543 (1990), the disclosures of which are hereby incorporated by reference. An oligonucleotide can include a label for detection, if desired.

Unless specified otherwise, the lefthand end of single-stranded polynucleotide sequences is the 5′ end; the lefthand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA and which are 5′ to the 5′ end of the RNA transcript are referred to as “upstream sequences”; sequence regions on the DNA strand having the same sequence as the RNA and which are 3′ to the 3′ end of the RNA transcript are referred to as “downstream sequences”.

“Operably linked” sequences include both expression control sequences that are contiguous with the gene of interest and expression control sequences that act in trans or at a distance to control the gene of interest. The term “expression control sequence” as used herein refers to polynucleotide sequences which are necessary to effect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include, at a minimum, all components whose presence is essential for expression and processing, and can also include additional components whose presence is advantageous, for example, leader sequences and fusion partner sequences.

The term “vector”, as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmid. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The term “recombinant host cell” (or simply “host cell”), as used herein, is intended to refer to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

The term “selectively hybridize” referred to herein means to detectably and specifically bind. Polynucleotides, oligonucleotides and fragments thereof in accordance with the invention selectively hybridize to nucleic acid strands under hybridization and wash conditions that minimize appreciable amounts of detectable binding to nonspecific nucleic acids. “High stringency” or “highly stringent” conditions can be used to achieve selective hybridization conditions as known in the art and discussed herein. An example of “high stringency” or “highly stringent” conditions is a method of incubating a polynucleotide with another polynucleotide, wherein one polynucleotide may be affixed to a solid surface such as a membrane, in a hybridization buffer of 6×SSPE or SSC, 50% formamide, 5×Denhardt's reagent, 0.5% SDS, 100 μg/ml denatured, fragmented salmon sperm DNA at a hybridization temperature of 42° C. for 12-16 hours, followed by twice washing at 55° C. using a wash buffer of 1×SSC, 0.5% SDS. See also Sambrook et al., supra, pp. 9.50-9.55.

The term “substantial homology or similarity” when referring to a nucleic acid or fragment thereof indicates that when optimally aligned (with appropriate nucleotide insertions or deletions) with the other nucleic acid (or its complementary strand), there is nucleotide sequence identity in at least about 60% of the nucleotide bases, usually at least about 70%, more usually at least about 80%, preferably at least about 90%, and more preferably at least about 95-98% of the nucleotide bases.

Alternatively, substantial homology or similarity exists when a nucleic acid or fragment thereof hybridizes to another nucleic acid (or a complementary strand thereof) under selective hybridization conditions, to a strand, or to its complement. Selectivity of hybridization exists when hybridization which is substantially more selective than total lack of specificity occurs. Typically, selective hybridization will occur when there is at least about 55% homology over a stretch of at least about 14 nucleotides, preferably at least about 60%, more preferably at least about 65%, more preferably at least about 75%, , more preferably at least about 80%, and most preferably at least about 90%. See, Kanehisa (1984) Nucl. Acids Res. 12:203-213. The length of homology comparison, as described, may be over longer stretches, and in certain embodiments will often be over a stretch of at least about nine nucleotides, usually at least about 20 nucleotides, more usually at least about 24 nucleotides, typically at least about 28 nucleotides, more typically at least about 32 nucleotides, and preferably at least about 36 or more nucleotides.

Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less are preferred with 2 or less being more preferred. Alternatively and preferably, two protein sequences (or polypeptide sequences derived from them of at least 30 amino acids in length) are homologous, as this term is used herein, if they have an alignment score of more than 5 (in standard deviation units) using the program ALIGN with the mutation data matrix and a gap penalty of 6 or greater. See Dayhoff, M. O., in Atlas of Protein Sequence and Structure, pp. 101-110 (Volume 5, National Biomedical Research Foundation (1972)) and Supplement 2 to this volume, pp. 1-10. The two sequences or parts thereof are more preferably homologous if their amino acids are greater than or equal to 50% identical when optimally aligned using the ALIGN program.

The term “corresponds to” is used herein to mean that a polynucleotide sequence is identical to all or a portion of a reference polynucleotide sequence, or that a polypeptide sequence is identical to a reference polypeptide sequence. In contrast, the term “complementary to” is used herein to mean that the complementary sequence is identical to all or a portion of a reference polynucleotide sequence. For illustration, the nucleotide sequence “TATAC” corresponds to a reference sequence “TATAC” and is complementary to a reference sequence “GTATA”.

The following terms are used to describe the sequence relationships between two or more polynucleotide or amino acid sequences: “reference sequence”, “comparison window”, “sequence identity”, “percentage of sequence identity”, and “substantial identity”. A “reference sequence” is a defined sequence used as a basis for a sequence comparison; a reference sequence may be a subset of a larger sequence, for example, as a segment of a full-length cDNA or gene sequence given in a sequence listing or may comprise a complete cDNA or gene sequence. Generally, a reference sequence is at least 18 nucleotides or 6 amino acids in length, frequently at least 24 nucleotides or 8 amino acids in length, and often at least 48 nucleotides or 16 amino acids in length. Since two polynucleotides or amino acid sequences may each (1) comprise a sequence (i.e., a portion of the complete polynucleotide or amino acid sequence) that is similar between the two molecules, and (2) may further comprise a sequence that is divergent between the two polynucleotides or amino acid sequences, sequence comparisons between two (or more) molecules are typically performed by comparing sequences of the two molecules over a “comparison window” to identify and compare local regions of sequence similarity. A “comparison window”, as used herein, refers to a conceptual segment of at least 18 contiguous nucleotide positions or 6 amino acids wherein a polynucleotide sequence or amino acid sequence may be compared to a reference sequence of at least 18 contiguous nucleotides or 6 amino acid sequences and wherein the portion of the polynucleotide sequence in the comparison window may comprise additions, deletions, substitutions, and the like (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman Adv. Appl. Math. 2:482 (1981), by the homology alignment algorithm of Needleman and Wunsch J. Mol. Biol. 48:443 (1970), by the search for similarity method of Pearson and Lipman Proc. Natl. Acad. Sci. U.S.A. 85:2444 (1988), by computerized implementations of these algorithms (GAP, BESTFIT, FASTA, and TFASTA in the Wisconsin Genetics Software Package Release 7.0, (Genetics Computer Group, 575 Science Dr., Madison, Wis.), Geneworks, or MacVector software packages), or by inspection, and the best alignment (i.e., resulting in the highest percentage of homology over the comparison window) generated by the various methods is selected.

The term “sequence identity” means that two polynucleotide or amino acid sequences are identical (i.e., on a nucleotide-by-nucleotide or residue-by-residue basis) over the comparison window.

The term “percentage of sequence identity” is calculated by comparing two optimally aligned sequences over the window of comparison, determining the number of positions at which the identical nucleic acid base (e.g., A, T, C, G, U, or I) or residue occurs in both sequences to yield the number of matched positions, dividing the number of matched positions by the total number of positions in the comparison window (i.e., the window size), and multiplying the result by 100 to yield the percentage of sequence identity. The terms “substantial identity” as used herein denotes a characteristic of a polynucleotide or amino acid sequence, wherein the polynucleotide or amino acid comprises a sequence that has at least 85 percent sequence identity, preferably at least 90 to 95 percent sequence identity, more preferably at least 98 percent sequence identity, more usually at least 99 percent sequence identity as compared to a reference sequence over a comparison window of at least 18 nucleotide (6 amino acid) positions, frequently over a window of at least 24-48 nucleotide (8-16 amino acid) positions, wherein the percentage of sequence identity is calculated by comparing the reference sequence to the sequence which may include deletions or additions which total 20 percent or less of the reference sequence over the comparison window. The reference sequence may be a subset of a larger sequence.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity, even more preferably at least 98 percent sequence identity and most preferably at least 99 percent sequence identity. Preferably, residue positions which are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, glutamate-aspartate, and asparagine-glutamine.

As discussed herein, minor variations in the amino acid sequences of antibodies or immunoglobulin molecules are contemplated as being encompassed by the present invention, providing that the variations in the amino acid sequence maintain at least 75%, more preferably at least 80%, 90%, 95%, and most preferably 99%. In particular, conservative amino acid replacements are contemplated. Conservative replacements are those that take place within a family of amino acids that are related in their side chains. Genetically encoded amino acids are generally divided into families: (1) acidic=aspartate, glutamate; (2) basic=lysine, arginine, histidine; (3) non-polar=alanine, valine, leucine, isoleucine, proline, phenylalanine, methionine, tryptophan; and (4) uncharged polar=glycine, asparagine, glutamine, cysteine, serine, threonine, tyrosine. More preferred families are: serine and threonine are aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Assays are described in detail herein.

As used herein, the terms “label” or “labeled” refers to incorporation of another molecule in the antibody. In one embodiment, the label is a detectable marker, e.g., incorporation of a radiolabeled amino acid or attachment to a polypeptide of biotinyl moieties that can be detected by marked avidin (e.g., streptavidin containing a fluorescent marker or enzymatic activity that can be detected by optical or calorimetric methods). In another embodiment, the label or marker can be therapeutic, e.g., a drug conjugate or toxin. Various methods of labeling polypeptides and glycoproteins are known in the art and may be used. Examples of labels for polypeptides include, but are not limited to, the following: radioisotopes or radionuclides (e.g., ³H, ¹⁴C , ¹⁵N, ³⁵S , ⁹⁰ Y, ⁹⁹Tc, ¹¹¹In, ¹²⁵I, ¹³¹I), fluorescent labels (e.g., FITC, rhodamine, lanthanide phosphors), enzymatic labels (e.g., horseradish peroxidase, β-galactosidase, luciferase, alkaline phosphatase), chemiluminescent markers, biotinyl groups, predetermined polypeptide epitopes recognized by a secondary reporter (e.g., leucine zipper pair sequences, binding sites for secondary antibodies, metal binding domains, epitope tags), magnetic agents, such as gadolinium chelates, toxins such as pertussis toxin, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. In some embodiments, labels are attached by spacer arms of various lengths to reduce potential steric hindrance.

The term “subject” includes human and non-human subjects (such as veterinary subjects). A patient is a subject.

The term “patient” includes human and veterinary subjects.

Throughout this specification and claims, the word “comprise,” or variations such as “comprises” or “comprising,” will be understood to imply the inclusion of a stated integer or group of integers but not the exclusion of any other integer or group of integers.

### Human Antibodies and Humanization of Antibodies

Human antibodies avoid certain of the problems associated with antibodies that possess mouse or rat variable and/or constant regions. The presence of such mouse or rat derived proteins can lead to the rapid clearance of the antibodies or can lead to the generation of an immune response against the antibody by a patient. In one embodiment, the invention provides humanized antibodies, or antigen binding portions thereof, that binds a mitochondrial antigen bound by a human autoantibody found in patients with PBC. In another embodiment, the invention provides fully human antibodies, or antigen binding portions thereof, that bind a mitochondrial antigen bound by a human autoantibody found in patients with PBC through the immunization of a rodent in which human immunoglobulin genes have been introduced so that the rodent produces fully human antibodies. Fully human antibodies are expected to minimize the immunogenic and allergic responses intrinsic to mouse or mouse-derivatized Mabs and thus to increase the efficacy and safety of the administered antibodies.

### Methods of Producing Antibodies and Antibody-producing Cell Lines

In one embodiment of the instant invention, human antibodies, or antigen binding portions thereof, are produced by immunizing a non-human animal comprising some or all of the human immunoglobulin locus with a mitochondrial antigen (could be, for example, a human mitochondrial antigen) bound by a human autoantibody found in patients with PBC. In a preferred embodiment, the non-human transgenic animal has the ability to make human antibodies, or antigen binding portions thereof, and is preferably deficient in the ability to make its cognate antibodies. In a preferred embodiment, the non-human animal is a mammal. In certain embodiments, the non-human animal is a mouse, goat, pig, etc. In other embodiments, the non-human animal is a XENOMOUSE® animal.

In a preferred embodiment, the mitochondrial antigen (in certain embodiments, the mitochondrial antigen is human mitochondrial antigen, but could be from other species, such as, for example, bovine) bound by a human autoantibody found in patients with PBC is selected from the group consisting of the E2 subunit of the pyruvate dehydrogenase complex, the E2 subunit of the branched-chain 2-oxo-acid dehydrogenase complex, the E2 subunit of the 2-oxoglutarate dehydrogenase complex, E3-binding protein and p100 bound by mAb 3E7.

XENOMOUSE® animal is any one of a number of engineered mouse strains that comprises large fragments of the human immunoglobulin loci (generally comprises some or all of the human heavy and light chain loci) and is deficient in mouse antibody production. See, e.g., Green et al. Nature Genetics 7:13-21 (1994) and U.S. Pat. Nos. 5,916,771, 5,939,598, 5,985,615, 5,998,209, 6,075,181, 6,091,001, 6,114,598, 6,130,364, 6,150,584 and 6,162,963. See also WO 91/10741, published Jul. 25, 1991, WO 94/02602, published Feb. 3, 1994, WO 96/34096 and WO 96/33735, both published Oct. 31, 1996, WO 98/16654, published Apr. 23, 1998, WO 98/24893, published Jun. 11, 1998, WO 98/50433, published Nov. 12, 1998, WO 99/45031, published Sep. 10, 1999, WO 99/53049, published Oct. 21, 1999, WO 00 09560, published Feb. 24, 2000 and WO 00/037504, published Jun. 29, 2000.

The first XENOMOUSE® animal strains were engineered with yeast artificial chromosomes (YACs) containing 245 kb and 190 kb-sized germline configuration fragments of the human heavy chain locus and kappa light chain locus, respectively, which contained core variable and constant region sequences. Id. A second generation XENOMOUSE® animals contain approximately 80% of the human antibody repertoire through introduction of megabase sized, germline configuration YAC fragments of the human heavy chain loci and kappa light chain loci. See Mendez et al. Nature Genetics 15:146-156 (1997), Green and Jakobovits J. Exp. Med. 188:483-495 (1998), and U.S. patent application Ser. No. 08/759,620, filed Dec. 3, 1996, the disclosures of which are hereby incorporated by reference. XENOMOUSE® animals produce an adult-like human repertoire of fully human antibodies, and generate antigen-specific human antibodies.

In another embodiment, the non-human animal comprising human immunoglobulin gene loci are animals that have a “minilocus” of human immunoglobulins. In the minilocus approach, an exogenous Ig locus is mimicked through the inclusion of individual genes from the Ig locus. Thus, one or more V_{H} genes, one or more D_{H} genes, one or more J_{H} genes, a mu constant region, and a second constant region (preferably a gamma constant region) are formed into a construct for insertion into an animal. This approach is described, inter alia, in U.S. Pat. Nos. 5,545,807, 5,545,806, 5,625,825, 5,625,126, 5,633,425, 5,661,016, 5,770,429, 5,789,650, 5,814,318, 5,591,669, 5,612,205, 5,721,367, 5,789,215, and 5,643,763, hereby incorporated by reference.

An advantage of the minilocus approach is the rapidity with which constructs, including portions of the Ig locus, can be generated and introduced into animals. However, one of the potential disadvantages of the minilocus approach is that there may not be sufficient immunoglobulin diversity to support full B-cell development, such that there may be lower antibody production.

In another embodiment, the invention provides a method for making anti-mitochondrial antigen antibodies (antibodies that bind one or more mitochondrial antigens bound by an autoantibody from a patient with PBC), or antigen binding portions thereof, in non-human animals other than mice by immunizing non-human transgenic animals that comprise human immunoglobulin loci and produce human heavy or light chains or the variable regions thereof. One may produce such animals using the methods described in U.S. Pat. Nos. 5,916,771, 5,939,598, 5,985,615, 5,998,209, 6,075,181, 6,091,001, 6,114,598 and 6,130,364. See also WO 91/10741, published Jul. 25, 1991, WO 94/02602, published Feb. 3, 1994, WO 96/34096 and WO 96/33735, both published Oct. 31, 1996, WO 98/16654, published Apr. 23, 1998, WO 98/24893, published Jun. 11, 1998, WO 98/50433, published Nov. 12, 1998, WO 99/45031, published Sep. 10, 1999, WO 99/53049, published Oct. 21, 1999, WO 00 09560, published Feb. 24, 2000 and WO 00/037504, published Jun. 29, 2000. The methods disclosed in these patents may modified as described in U.S. Pat. No. 5,994,619. In preferred embodiments, the non-human animals may be rats, sheep, pigs, goats, cattle or horses.

In another embodiment, the invention provides a method for making antibodies to a mitochondrial antigen bound by a human autoantibody found in patients with PBC from non-human animals. In this embodiment, the non-human animals are immunized with said antigen and non-human antibodies are produced by these animals. Antibody-producing cells may be isolated from these animals, immortalized by any means known in the art, for example, preferably by fusion with myelomas to produce hybridomas, and subsequently engineered to produce “humanized antibodies” such that they do not cause an immune response in a human using techniques known to those of skill in the art and as described further below.

An isolated human autoantibody to a mitochondrial antigen can be isolated from a patient with PBC by conventional techniques.

This invention also provides a method of making an isolated human monoclonal antibody, or antigen-binding portions thereof, that binds a mitochondrial antigen bound by a human autoantibody found in patients with primary biliary cirrhosis comprising the steps of:

a) providing a transgenic non-human animal that has the ability to make human antibodies (preferably deficient in the ability to make said animal's antibodies);

b) immunizing said animal with said mitochondrial antigen;

c) obtaining a hybridoma cell line making a human antibody to said antigen; and

d) isolating said human antibody to said antigen.

In certain embodiments, said transgenic non-human animal is a mouse. In certain embodiments, said mouse is a XENOMOUSE® mouse. In certain embodiments, said mitochondrial antigen is selected from the group consisting of the E2 subunit of the pyruvate dehydrogenase complex, the E2 subunit of the branched-chain 2-oxo-acid dehydrogenase complex, the E2 subunit of the 2-oxoglutarate dehydrogenase complex, E3-binding protein and p100 bound by mAb 3E7. In certain embodiments, said isolated human antibody binds to one or more peptide, polypeptide or protein selected from the group consisting of the inner lipoyl domain of the E2 subunit of pyruvate dehydrogenase complex; the E2 subunit of the pyruvate dehydrogenase complex; the E2 subunit of the 2-oxoglutarate dehydrogenase complex; the E2 subunit of the branched-chain 2-oxo-acid dehydrogenase complex, MIT3 and p100. Said one ore more peptide, polypeptide or protein may be part of a fusion protein. Said one ore more peptide, polypeptide or protein may be affinity purified by an antibody of this invention or by autoantibody from patients with PBC, by methods well known in the art.

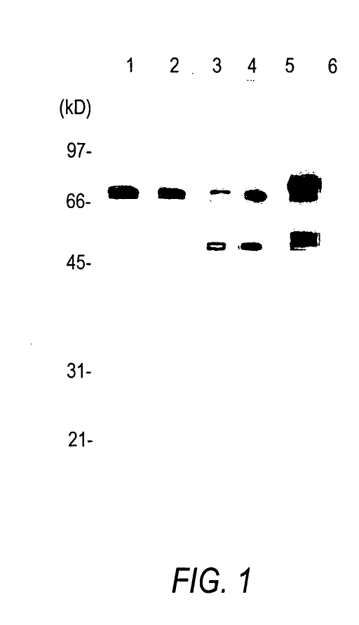

This invention also provides an isolated antibody (which could be a human antibody; which could be a monoclonal antibody; which could be a human monoclonal antibody), or antigen-binding portions thereof, that binds a mitochondrial antigen bound by a human autoantibody, or antigen-binding portions thereof, found in patients with primary biliary cirrhosis. In certain embodiments, the isolated human antibody, or antigen binding portions thereof, is produced by any one of the methods of this invention, such as the methods described in the preceding paragraph. In certain embodiments, said human antibody is an IgG2 or an IgM antibody. In certain embodiments, said antibody has one or more CDR1, CDR2 or CDR3 of any one of the amino acid sequence of: SEQ ID NO: 1, SEQ ID NO: 2, SEQ ID NO: 3, SEQ ID NO: 6, SEQ ID NO: 7, SEQ ID NO: 8, SEQ ID NO: 9 or SEQ ID NO: 10 (in certain embodiments, said antibody has a CDR3 of any one of the amino acid sequence of: SEQ ID NO: 1, SEQ ID NO: 2, SEQ ID NO: 3, SEQ ID NO: 6, SEQ ID NO: 7, SEQ ID NO: 8, SEQ ID NO: 9 or SEQ ID NO: 10). In certain embodiments, said antibody has one or more non-germline FR1, FR2, FR3 or FR4 of any one of the amino acid sequence of: SEQ ID NO: 1, SEQ ID NO: 2, SEQ ID NO: 3, SEQ ID NO: 6, SEQ ID NO: 7, SEQ ID NO: 8, SEQ ID NO: 9 or SEQ ID NO: 10. In certain embodiments, said antibody comprises a heavy chain that utilizes a V_{H} gene selected from the group consisting of V_{H}3, V_{H}4 and V_{H}6. In certain embodiments, said antibody is one of the thirteen human monoclonal antibodies, or antigen-binding portions thereof, described in the Example section (which includes Examples 1-3) and FIGS. 1-8, that binds one or more mitochondrial antigens bound by autoantibody from a patient with PBC. These thirteen monoclonal antibodies are: 2E6, 4D11, 1C7, 2E11, 2C8, 4C2, 3B4, 1F8, 3D8, 4B10, 4G6, 2G8 and 3E7. In certain embodiments, said antibody comprises a heavy chain that has at least one mutation compared to the germline heavy chain. In certain embodiments, said isolated antibody comprises a heavy chain that utilizes a V_{H}3 gene selected from the group consisting of V_{H}3-23, V_{H}3-21, V_{H}3-33, V_{H}3-7 and V_{H}3-53. In certain embodiments, said isolated antibody comprises a heavy chain that utilizes a V_{H}4-61 gene. In certain embodiments, said isolated antibody comprises a heavy chain that utilizes a V_{H}6-1 gene. In certain embodiments, said isolated antibody comprises a light chain that utilizes a V_{K} gene selected from the group consisting of V_{K}1-12, V_{K}1-17, V_{K}1-27, V_{K}1-39, V_{K}2-28, V_{K}2-30, V_{K}4-1. In certain embodiments, said isolated antibody comprises a light chain that has at least one mutation compared to the germline light chain.

This invention also provides an isolated antibody (could be a human antibody; could be a monoclonal antibody; could be a human monoclonal antibody)(in certain embodiments, said antibody binds to one or more mitochondrial antigens bound by an autoantibody from patients with PBC) or antigen-binding portion thereof, wherein said antibody comprises a heavy chain VDJ region amino acid sequence and a light chain VJ region amino acid sequence selected from the group consisting of:

a. a heavy chain VDJ region amino acid sequence encoded by the nucleic acid sequence of SEQ ID NO: 28, and a light chain VJ region amino acid sequence encoded by the nucleic acid sequence of SEQ ID NO: 12, or said amino acid sequences encoded by said nucleic acid sequences encoding the amino acid sequences lacking the signal sequences;

(b) a heavy chain VDJ region amino acid sequence encoded by the nucleic acid sequence of SEQ ID NO: 29, and a light chain VJ region amino acid sequence encoded by the nucleic acid sequence of SEQ ID NO: 13, or said amino acid sequences encoded by said nucleic acid sequences encoding the amino acid sequences lacking the signal sequences;

(c) a heavy chain VDJ region amino acid sequence encoded by the nucleic acid sequence of SEQ ID NO: 24, and a light chain VJ region amino acid sequence encoded by the nucleic acid sequence of SEQ ID NO: 14, or said amino acid sequences encoded by said nucleic acid sequences encoding the amino acid sequences lacking the signal sequences;

(d) a heavy chain VDJ region amino acid sequence encoded by the nucleic acid sequence of SEQ ID NO: 26, and a light chain VJ region amino acid sequence encoded by the nucleic acid sequence of SEQ ID NO: 16, or said amino acid sequences encoded by said nucleic acid sequences encoding the amino acid sequences lacking the signal sequences;

(e) a heavy chain VDJ region amino acid sequence encoded by the nucleic acid sequence of SEQ ID NO: 32, and a light chain VJ region amino acid sequence encoded by the nucleic acid sequence of SEQ ID NO: 17, or said amino acid sequences encoded by said nucleic acid sequences encoding the amino acid sequences lacking the signal sequences;

(f) a heavy chain VDJ region amino acid sequence encoded by the nucleic acid sequence of SEQ ID NO: 25, and a light chain VJ region amino acid sequence encoded by the nucleic acid sequence of SEQ ID NO: 18, or said amino acid sequences encoded by said nucleic acid sequences encoding the amino acid sequences lacking the signal sequences;

(g) a heavy chain VDJ region amino acid sequence encoded by the nucleic acid sequence of SEQ ID NO: 22, and a light chain VJ region amino acid sequence encoded by the nucleic acid sequence of SEQ ID NO: 19, or said amino acid sequences encoded by said nucleic acid sequences encoding the amino acid sequences lacking the signal sequences;

(h) a heavy chain VDJ region amino acid sequence encoded by the nucleic acid sequence of SEQ ID NO: 23, and a light chain VJ region amino acid sequence encoded by the nucleic acid sequence of SEQ ID NO: 20, or said amino acid sequences encoded by said nucleic acid sequences encoding the amino acid sequences lacking the signal sequences; and

(i) a heavy chain VDJ region amino acid sequence encoded by the nucleic acid sequence of SEQ ID NO: 27, and a light chain VJ region amino acid sequence encoded by the nucleic acid sequence of SEQ ID NO: 21, or said amino acid sequences encoded by said nucleic acid sequences encoding the amino acid sequences lacking the signal sequences.

This invention also provides an isolated antibody (could be a human antibody; could be a monoclonal antibody; could be a human monoclonal antibody) (in certain embodiments, said antibody binds to one or more mitochondrial antigens bound by an autoantibody from patients with PBC) or antigen-binding portion thereof, wherein said antibody comprises a heavy chain VDJ region amino acid sequence selected from the group consisting of:

a. a heavy chain VDJ region amino acid sequence encoded by the nucleic acid sequence of SEQ ID NO: 30, or said amino acid sequence encoded by said nucleic acid sequence encoding the amino acid sequence lacking the signal sequence; and

b. a heavy chain VDJ region amino acid sequence encoded by the nucleic acid sequence of SEQ ID NO: 31, or said amino acid sequence encoded by said nucleic acid sequence encoding the amino acid sequence lacking the signal sequence.

This invention also provides an isolated antibody (could be a human antibody; could be a monoclonal antibody; could be a human monoclonal antibody) (in certain embodiments, said antibody binds to one or more mitochondrial antigens bound by an autoantibody from patients with PBC) or antigen-binding portion thereof, wherein said antibody comprises a light chain VJ region amino acid sequence encoded by the nucleic acid sequence of SEQ ID NO: 15, or said amino acid sequence encoded by said nucleic acid sequence encoding the amino acid sequence lacking the signal sequence.

This invention also provides an isolated antibody (could be a human antibody; could be a monoclonal antibody; could be a human monoclonal antibody) (in certain embodiments, said antibody binds to one or more mitochondrial antigens bound by an autoantibody from patients with PBC) or antigen-binding portion thereof, wherein said antibody comprises a light chain VJ region amino acid sequence encoded by the nucleic acid sequence of SEQ ID NO: 15, wherein said antibody comprises a heavy chain that utilizes a V_{H}3-23 gene, a D6-19 gene and a J_{H}6 gene and wherein said heavy chain has at least one mutation compared to the germline heavy chain, and wherein said antibody specifically binds to the E2 component of the pyruvate dehydrogenase complex (PDC-E2) and the inner lipoyl domain of PDC-E2.

This invention also provides an isolated antibody (could be a human antibody; could be a monoclonal antibody; could be a human monoclonal antibody) (in certain embodiments, said antibody binds to one or more mitochondrial antigens bound by an autoantibody from patients with PBC) or antigen-binding portion thereof, wherein said antibody comprises a heavy chain VDJ region amino acid sequence encoded by the nucleic acid sequence of SEQ ID NO: 30, wherein said antibody comprises a light chain that utilizes a V_{K}1-17 gene and a J_{K}2 gene and wherein said light chain has at least one mutation compared to the germline light chain and wherein said antibody does not specifically binds to the E2 component of the pyruvate dehydrogenase complex (PDC-E2) but binds specifically to the E2 component of the 2-oxoglutarate dehydrogenase complex (OGDC-E2).

This invention also provides an isolated antibody (could be a human antibody; could be a monoclonal antibody; could be a human monoclonal antibody)(in certain embodiments, said antibody binds to one or more mitochondrial antigens bound by an autoantibody from patients with PBC) or antigen-binding portion thereof, wherein said antibody comprises a heavy chain VDJ region amino acid sequence encoded by the nucleic acid sequence of SEQ ID NO: 31, wherein said antibody comprises a light chain that utilizes a V_{K}2-30 gene and a J_{K}5 gene and wherein said antibody does not specifically binds to the E2 component of the pyruvate dehydrogenase complex (PDC-E2) but binds specifically to the E2 component of the branched-chain 2-oxo-acid dehydrogenase complex (BCOADC-E2).

This invention also provides an isolated antibody (could be a human antibody; could be a monoclonal antibody; could be a human monoclonal antibody)(in certain embodiments, said antibody binds to one or more mitochondrial antigens bound by an autoantibody from patients with PBC) or antigen-binding portion thereof, wherein said antibody comprises a light chain that utilizes a V_{K}1-39 gene and a J_{K}3 gene and wherein said light chain has at least one mutation compared to the germline light chain, wherein said antibody comprises a heavy chain that utilizes a V_{H}3-7 gene, a D6-19 gene and a J_{H}6 gene and wherein said heavy chain has at least one mutation compared to the germline heavy chain and wherein said antibody specifically binds to the E2 component of the pyruvate dehydrogenase complex (PDC-E2) but does not specifically bind to the inner lipoyl domain of PDC-E2. 30.

This invention also provides an isolated antibody (could be a human antibody; could be a monoclonal antibody; could be a human monoclonal antibody)(in certain embodiments, said antibody binds to one or more mitochondrial antigens bound by an autoantibody from patients with PBC) or antigen-binding portion thereof, wherein said antibody comprises a light chain VJ region amino acid sequence encoded by the nucleic acid sequence of SEQ ID NO: 15 and wherein said antibody comprises a heavy chain VDJ region amino acid sequence of SEQ ID NO: 33.

This invention also provides an isolated antibody (could be a human antibody; could be a monoclonal antibody; could be a human monoclonal antibody)(in certain embodiments, said antibody binds to one or more mitochondrial antigens bound by an autoantibody from patients with PBC) or antigen-binding portions thereof, wherein said antibody comprises a heavy chain VDJ region amino acid sequence encoded by the nucleic acid sequence of SEQ ID NO: 30 and wherein said antibody comprises a light chain VJ region amino acid sequence of SEQ ID NO: 36.

This invention also provides an isolated antibody (could be a human antibody; could be a monoclonal antibody; could be a human monoclonal antibody)(in certain embodiments, said antibody binds to one or more mitochondrial antigens bound by an autoantibody from patients with PBC) or antigen-binding portion thereof, wherein said antibody comprises a heavy chain VDJ region amino acid sequence encoded by the nucleic acid sequence of SEQ ID NO: 31 and wherein said antibody comprises a light chain VJ region amino acid sequence of SEQ ID NO: 37.

This invention also provides an isolated antibody (could be a human antibody; could be a monoclonal antibody; could be a human monoclonal antibody)(in certain embodiments, said antibody binds to one or more mitochondrial antigens bound by an autoantibody from patients with PBC) or antigen-binding portion thereof, wherein said antibody comprises a heavy chain VDJ region amino acid sequence of SEQ ID NO: 34 and wherein said antibody comprises a light chain VJ region amino acid sequence of SEQ ID NO: 35.

This invention also provides an isolated antibody (could be a human antibody; could be a monoclonal antibody; could be a human monoclonal antibody) (in certain embodiments, said antibody binds to one or more mitochondrial antigens bound by an autoantibody from patients with PBC), or antigen-binding portions thereof, comprising a light chain comprising a CDR3-junction amino acid sequence selected from the group consisting of: a CDR3-junction amino acid sequence of SEQ ID NO: 38, a CDR3-junction amino acid sequence of SEQ ID NO: 39, a CDR3-junction amino acid sequence of SEQ ID NO: 40, a CDR3-junction amino acid sequence of SEQ ID NO: 41, a CDR3-junction amino acid sequence of SEQ ID NO: 42, a CDR3-junction amino acid sequence of SEQ ID NO: 43, a CDR3-junction amino acid sequence of SEQ ID NO: 44, a CDR3-junction amino acid sequence of SEQ ID NO: 45 and a CDR3-junction amino acid sequence of SEQ ID NO: 46.

This invention also provides an isolated antibody (could be a human antibody; could be a monoclonal antibody; could be a human monoclonal antibody) (in certain embodiments, said antibody binds to one or more mitochondrial antigens bound by an autoantibody from patients with PBC), or antigen-binding portions thereof, having a non-germline heavy chain region of any one of the amino acid sequence of: SEQ ID NO: 1, SEQ ID NO: 2 or SEQ ID NO: 3. This invention also provides an isolated antibody (could be a human antibody; could be a monoclonal antibody; could be a human monoclonal antibody) (in certain embodiments, said antibody binds to one or more mitochondrial antigens bound by an autoantibody from patients with PBC), or antigen-binding portions thereof, having a non-germline light chain region of any one of the amino acid sequence of: SEQ ID NO: 6, SEQ ID NO: 7, SEQ ID NO: 8, SEQ ID NO: 9 or SEQ ID NO: 10.

This invention also provides a DNA molecule encoding an immunoglobulin comprising an amino acid sequence of SEQ ID NO: 1, SEQ ID NO: 2, SEQ ID NO: 3, SEQ ID NO: 6, SEQ ID NO: 7, SEQ ID NO: 8, SEQ ID NO: 9 or SEQ ID NO: 10.

This invention also provides a cell line, such as a hybridoma cell line, that secretes a human antibody, or antigen-binding portions thereof, or immunoglobulin of this invention.

This invention also provides an isolated antibody (could be a human antibody; could be a monoclonal antibody; could be a human monoclonal antibody)(in certain embodiments, said antibody binds to one or more mitochondrial antigens bound by an autoantibody from patients with PBC), or antigen-binding portions thereof, having a non-germline heavy chain region of any one of the amino acid sequence selected from the group consisting of: SEQ ID NO: 1, SEQ ID NO: 2, SEQ ID NO: 3, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63 and SEQ ID NO: 64.

This invention also provides an isolated antibody (could be a human antibody; could be a monoclonal antibody; could be a human monoclonal antibody)(in certain embodiments, said antibody binds to one or more mitochondrial antigens bound by an autoantibody from patients with PBC), or antigen-binding portions thereof, having a non-germline light chain region of any one of the amino acid sequence selected from the group consisting of: SEQ ID NO: 6, SEQ ID NO: 7, SEQ ID NO: 8, SEQ ID NO: 9, SEQ ID NO: 10, SEQ ID NO: 47, SEQ ID NO: 48, SEQ ID NO: 49, SEQ ID NO: 50, SEQ ID NO: 51, SEQ ID NO: 52, SEQ ID NO: 53, and SEQ ID NO: 54.

This invention also provides an isolated antibody or antigen-binding portion thereof, wherein said antibody comprises a heavy chain VDJ amino acid sequence and a light chain VJ amino acid sequence selected from the group consisting of:

(a) a heavy chain VDJ region amino acid sequence of SEQ ID NO: 1, and a light chain VJ region amino acid sequence encoded by the nucleic acid sequence of SEQ ID NO: 8, or said amino acid sequences lacking the signal sequences;

(b) a heavy chain VDJ region amino acid sequence of SEQ ID NO: 2, and a light chain VJ region amino acid sequence encoded by the nucleic acid sequence of SEQ ID NO: 9, or said amino acid sequences lacking the signal sequences;

(c) a heavy chain VDJ region amino acid sequence of SEQ ID NO: 3, and a light chain VJ region amino acid sequence encoded by the nucleic acid sequence of SEQ ID NO: 10, or said amino acid sequences lacking the signal sequences;

(d) a heavy chain VDJ region amino acid sequence of SEQ ID NO: 55, and a light chain VJ region amino acid sequence encoded by the nucleic acid sequence of SEQ ID NO: 6, or said amino acid sequences lacking the signal sequences;

(e) a heavy chain VDJ region amino acid sequence of SEQ ID NO: 56, and a light chain VJ region amino acid sequence encoded by the nucleic acid sequence of SEQ ID NO: 47, or said amino acid sequences lacking the signal sequences;

(f) a heavy chain VDJ region amino acid sequence of SEQ ID NO: 57, and a light chain VJ region amino acid sequence encoded by the nucleic acid sequence of SEQ ID NO: 7, or said amino acid sequences lacking the signal sequences;

(g) a heavy chain VDJ region amino acid sequence of SEQ ID NO: 58, and a light chain VJ region amino acid sequence encoded by the nucleic acid sequence of SEQ ID NO: 53, or said amino acid sequences lacking the signal sequences;

(h) a heavy chain VDJ region amino acid sequence of SEQ ID NO: 59, and a light chain VJ region amino acid sequence encoded by the nucleic acid sequence of SEQ ID NO: 54, or said amino acid sequences lacking the signal sequences;

(i) a heavy chain VDJ region amino acid sequence of SEQ ID NO: 60, and a light chain VJ region amino acid sequence encoded by the nucleic acid sequence of SEQ ID NO: 51, or said amino acid sequences lacking the signal sequences;

(j) a heavy chain VDJ region amino acid sequence of SEQ ID NO: 61, and a light chain VJ region amino acid sequence encoded by the nucleic acid sequence of SEQ ID NO: 48, or said amino acid sequences lacking the signal sequences;

(k) a heavy chain VDJ region amino acid sequence of SEQ ID NO: 62, and a light chain VJ region amino acid sequence encoded by the nucleic acid sequence of SEQ ID NO: 52, or said amino acid sequences lacking the signal sequences;

(l) a heavy chain VDJ region amino acid sequence of SEQ ID NO: 63 and a light chain VJ region amino acid sequence encoded by the nucleic acid sequence of SEQ ID NO: 50, or said amino acid sequences lacking the signal sequences; and

(m) a heavy chain VDJ region amino acid sequence of SEQ ID NO: 64, and a light chain VJ region amino acid sequence encoded by the nucleic acid sequence of SEQ ID NO: 49, or said amino acid sequences lacking the signal sequences.

This invention also provides a transgenic, non-human animal that produces a human antibody, or antigen-binding portions thereof, or immunoglobulin of this invention.

This invention also provides a method of identifying a potential antagonist of a human antibody (the antibody binds to one or more peptide, polypeptide or protein of, for example, the inner lipoyl domain of the E2 subunit of pyruvate dehydrogenase complex, the E2 subunit of the pyruvate dehydrogenase complex, the E2 subunit of the 2-oxoglutarate dehydrogenase complex, the E2 subunit of the branched-chain 2-oxo-acid dehydrogenase complex, MIT3 and p100 (bound by mAb 3E7); in certain embodiments, the peptide, polypeptide or protein is of human origin) that binds to a mitochondrial antigen (the mitochondrial antigen (could be, for example, human mitochondrial antigen) could be, for example, the E2 subunit of the pyruvate dehydrogenase complex, the E2 subunit of the branched-chain 2-oxo-acid dehydrogenase complex, the E2 subunit of the 2-oxoglutarate dehydrogenase complex and E3-binding protein) bound by a human autoantibody found in patients with primary biliary cirrhosis, comprising the step of screening for a potential antagonist in a non-human transgenic animal (could be a mouse, which could be a XENOMOUSE® mouse) that has the ability to make human antibodies and is preferably deficient in the ability to make its cognate antibodies, wherein said potential antagonist reduces the production of said human antibody in said transgenic non-human animal. The human antibody may be detected in a number of ways well known in the art, including detecting its levels in the sera of the transgenic non-human animal by ELISA or RIA.

This invention also provides a method of treating or preventing primary biliary cirrhosis, comprising the step of modifying or blocking the epitope on some or all mitochondrial antigens bound by a human antibody that binds to mitochondrial antigens bound by a human autoantibody found in patients with primary biliary cirrhosis.

### Production of Monoclonal Antibodies and Antibody-producing Cell Lines

Antigen

The antigen for generating an antibody of this invention is any mitochondrial antigen bound by an autoantibody from patients with PBC. In certain embodiments, said mitochondrial antigen is selected from the group consisting of the E2 subunit of the pyruvate dehydrogenase complex, the E2 subunit of the branched-chain 2-oxo-acid dehydrogenase complex, the E2 subunit of the 2-oxoglutarate dehydrogenase complex, E3-binding protein and p100 bound by mAb 3E7. Said antigen may be part of a fusion protein. Said antigen may be affinity purified by an antibody of this invention or by autoantibody from patients with PBC, by methods well known in the art.

Immunization

Immunization of animals may be done by any method known in the art. See, e.g., Harlow and Lane, Antibodies: A Laboratory Manual, New York: Cold Spring Harbor Press, 1990. Methods for immunizing non-human animals such as mice, rats, sheep, goats, pigs, cattle and horses are well known in the art. See, e.g., Harlow and Lane and U.S. Pat. No. 5,994,619. In a preferred embodiment, the antigen is administered with or without an adjuvant to stimulate the immune response. Such adjuvants include, inter alia, complete or incomplete Freund's adjuvant, RIBI (muramyl dipeptides) or ISCOM (immunostimulating complexes). Such adjuvants may protect the polypeptide from rapid dispersal by sequestering it in a local deposit, or they may contain substances that stimulate the host to secrete factors that are chemotactic for macrophages and other components of the immune system. Preferably, if a polypeptide is being administered, the immunization schedule will involve two or more administrations of the polypeptide, spread out over several weeks.

After immunization of an animal with an antigen, such as a mitochondrial antigen bound by an auto-antibody from patients with PBC, antibodies and/or antibody-producing cells may be obtained from the animal. In one embodiment, antibody-containing serum is obtained from the animal by bleeding or sacrificing the animal. The serum may be used as it is obtained from the animal, an immunoglobulin fraction may be obtained from the serum, or the antibodies may be purified from the serum. It is well known to one of ordinary skill in the art that serum or immunoglobulins obtained in this manner will be polyclonal. The disadvantage is using polyclonal antibodies prepared from serum is that the amount of antibodies that can be obtained is limited and the polyclonal antibody has a heterogeneous array of properties.

In another embodiment, antibody-producing immortalized cells may be prepared from antibody producing cells isolated from the immunized animal. After immunization, the animal is sacrificed and the antibody producing cells, such as splenic B cells, are immortalized by any means known in the art. For example, the antibody producing cells may be immortalized by fusion with immortalized myeloma cells as is well-known in the art to produce hybridomas. See. e.g., Harlow and Lane, supra. In a preferred embodiment, the myeloma cells do not secrete cognate immunoglobulin polypeptides (non-secretory cell-lines). After fusion and antibiotic selection, the immortalized cells, such as hybridomas, are screened using a mitochondrial antigen bound by a human autoantibody found in patients with PBC. In a preferred embodiment, the initial screening is performed using an enzyme-linked immunoassay (ELISA) or a radioimmunoassay. In a more preferred embodiment, an ELISA is used for initial screening. An example of ELISA screening is provided in WO 00/37504, the disclosure of which is herein incorporated by reference.

Antibody-producing and secreting immortalized cells, such as hybridomas, are selected, cloned and further screened for desirable characteristics, including robust growth, high antibody production and desirable antibody characteristics, as discussed further below. Hybridomas may be expanded in vivo in syngeneic animals, in animals that lack an immune system, e.g., nude mice, or in cell culture in vitro. Methods of selecting, cloning and expanding immortalized cells, such as hybridomas, are well known to those of ordinary skill in the art.

In a preferred embodiment, the immunized animal is a non-human animal that expresses human immunoglobulin genes and the splenic B cells are fused to a myeloma derived from the same species as the non-human animal. In a more preferred embodiment, the immunized animal is a XENOMOUSE® animal and the myeloma cell line is a non-secretory mouse myeloma.

In one embodiment, hybridomas are produced that produce human antibodies to a mitochondrial antigen bound by a human autoantibody found in patients with PBC. In a preferred embodiment, the hybridomas are mouse hybridomas, as described above. In another preferred embodiment, the hybridomas are produced in a non-human, non-mouse species such as rats, sheep, pigs, goats, cattle or horses. In another embodiment, the hybridomas are human hybridomas, in which a human non-secretory myeloma is fused with a human cell expressing an antibody to a mitochondrial antigen bound by a human autoantibody found in patients with PBC.

In another embodiment, antibody-producing cells may be prepared from a human who has PBC and who expresses antibodies to one or more mitochondrial antigens bound by a human autoantibody found in patients with PBC. Cells expressing the antibodies to one or more mitochondrial antigens bound by a human autoantibody found in patients with PBC may be isolated by isolating white blood cells and subjecting them to fluorescence-activated cell sorting (FACS) or by panning on plates coated with mitochondrial antigens, or a portion thereof, bound by a human autoantibody found in patients with PBC. These cells may be fused with a human non-secretory myeloma to produce human hybridomas expressing human antibodies to one or more mitochondrial antigens bound by a human autoantibody found in patients with PBC.

An isolated human monoclonal antibody bound by a human autoantibody found in patients with PBC described herein may be used, for example, to purify the antigen to which it binds, to produce an anti-idiotype antibody to it, as a reagent to screen for potential antagonists to it, either in vivo or in vitro.

### Nucleic Acids, Vectors, Host Cells and Recombinant Methods of Making Antibodies

A nucleic acid molecule encoding either the entire heavy chain and light chain of an antibody to a mitochondrial antigen bound by a human autoantibody found in patients with PBC or the variable regions thereof may be obtained from any source that produces such an antibody.

A nucleic acid molecule could be, for example, a DNA molecule, a RNA molecule, etc. A DNA may be, for example, single stranded or double stranded. Due to base-pairing complementarity, a single stranded nucleic acid can be converted into a double stranded nucleic acid. Also, the complementary strand of a nucleic acid can be obtained if that nucleic acid is provided.

In one embodiment of the invention, such nucleic acid molecules may be obtained from a hybridoma that expresses an antibody, such as from one of the hybridomas described above. Methods of isolating mRNA encoding immunoglobulins are well-known in the art. See, e.g., Sambrook et al., supra. The mRNA may be used to produce cDNA for use in the polymerase chain reaction (PCR) or cDNA cloning of antibody genes. In a preferred embodiment, the nucleic acid molecule is derived from a hybridoma that has as one of its fusion partners a non-human animal cell that expresses human immunoglobulin genes. In an even more preferred embodiment, the fusion partner animal cell is derived from a XENOMOUSE® animal. In another embodiment, the hybridoma is derived from a non-human transgenic animal other than mouse as described above. In another embodiment, the hybridoma is derived from a non-human, non-transgenic animal. The nucleic acid molecules derived from a non-human, non-transgenic animal may be used, e.g., for humanized antibodies.

In a preferred embodiment, the heavy chain of an antibody to a mitochondrial antigen bound by a human autoantibody found in patients with PBC may be constructed by fusing a nucleic acid molecule encoding the variable domain of a heavy chain with a constant domain of a heavy chain. Similarly, the light chain of an antibody to a mitochondrial antigen bound by a human autoantibody found in patients with PBC may be constructed by fusing a nucleic acid molecule encoding the variable domain of a light chain with a constant domain of a light chain.

In another embodiment, an antibody to a mitochondrial antigen bound by a human autoantibody found in patients with PBC producing cell itself may be purified from a non-human animal. In one embodiment, the antibody-producing cells may be derived from a transgenic non-human animal that expresses human immunoglobulin genes and has been immunized with a suitable antigen. The transgenic non-human animal may be a mouse, such as a XENOMOUSE® animal, or another non-human transgenic animal. In another embodiment, the antibody to a mitochondrial antigen bound by a human autoantibody found in patients with PBC producing cell is derived from a non-transgenic animal. In another embodiment, the antibody to a mitochondrial antigen bound by a human autoantibody found in patients with PBC producing cell may be derived from a human patient with PBC who produces antibodies to one or more mitochondrial antigens bound by a human autoantibody found in patients with PBC. The mRNA from the antibody-producing cells may be isolated by standard techniques, amplified using PCR and screened using standard techniques to obtain nucleic acid molecules encoding anti-mitochondrial antigen (i.e., anti-mitochondrial antigen bound by a human autoantibody found in patients with PBC) heavy and light chains, or antigen-binding portions thereof.

In another embodiment, the nucleic acid molecules may be used to make vectors using methods known to those having ordinary skill in the art. See, e.g., Sambrook et al., supra, and Ausubel et al., supra. In one embodiment, the vectors may be plasmid or cosmid vectors. In another embodiment, the vectors may be viral vectors. Viral vectors include, without limitation, adenovirus, retrovirus, adeno-associated viruses and other picorna viruses, hepatitis virus and baculovirus. The vectors may also be bacteriophage including, without limitation, M13.

The nucleic acid molecules may be used to recombinantly express large quantities of antibodies, as described below. The nucleic acid molecules also may be used to produce chimeric antibodies, single chain antibodies, immunoadhesins, diabodies, mutated antibodies and antibody derivatives, as described further below. If the nucleic acid molecules encode non-human antibodies, the nucleic acid molecules may be used for antibody humanization, also as described below.

In one embodiment, the nucleic acid molecules encoding the variable region of the heavy (V_{H}) and light (V_{L}) chains are converted to full-length antibody genes. In one embodiment, the nucleic acid molecules encoding the V_{H} and V_{L} chain are converted to full-length antibody genes by inserting them into expression vectors already encoding heavy chain constant (C_{H}) and light chain constant (C_{L}) regions, respectively, such that the V_{H} is operatively linked to the C_{H} within the vector and the V_{L} is operatively linked to the C_{L} within the vector. In another embodiment, the nucleic acid molecules encoding the VH and/or VL are converted into full-length antibody genes by linking the nucleic acid molecule encoding a VH to a nucleic acid molecule encoding a CH using standard molecular biological techniques. The same may be achieved using nucleic acid molecules encoding VL and CL. The sequences of human heavy and light chain constant region genes are known in the art. See, e.g., Kabat et al., Sequences of Proteins of Immunological Interest, 5th Ed., NIH Publ. No. 91-3242, 1991.

In another embodiment, the nucleic acid molecules of the invention may be used as probes or PCR primers for specific antibody sequences. For instance, a nucleic acid molecule probe may be used in diagnostic methods or a nucleic acid molecule PCR primer may be used to amplify regions of DNA that could be used, inter alia, to isolate nucleic acid sequences for use in producing variable domains of the antibodies of the present invention. In a preferred embodiment, the nucleic acid molecules are oligonucleotides. In a more preferred embodiment, the oligonucleotides are from highly variable regions of the heavy and light chains of the antibody of interest. In an even more preferred embodiment, the oligonucleotides encode all or a part of one or more of the CDRs.

This invention provides a DNA molecule encoding an immunoglobulin comprising a amino acid sequence of any one of the amino acid sequence selected from the group consisting of: SEQ ID NO: 1, SEQ ID NO: 2, SEQ ID NO: 3, SEQ ID NO: 6, SEQ ID NO: 7, SEQ ID NO: 8, SEQ ID NO: 9 and SEQ ID NO: 10.

This invention also provides a DNA molecule encoding an immunoblogulin (in certain embodiments, said immunoglobulin binds to one or more mitochondrial antigens bound by an autoantibody from patients with PBC) comprising a heavy chain VDJ region amino acid sequence and light chain VJ region amino acid sequence selected from the group consisting of:

(a) a heavy chain VDJ region amino acid sequence encoded by the nucleic acid sequence of SEQ ID NO: 28, and a light chain VJ region amino acid sequence encoded by the nucleic acid sequence of SEQ ID NO: 12, or said amino acid sequences encoded by said nucleic acid sequences encoding the amino acid sequences lacking the signal sequences;

(b) a heavy chain VDJ region amino acid sequence encoded by the nucleic acid sequence of SEQ ID NO: 29, and a light chain VJ region amino acid sequence encoded by the nucleic acid sequence of SEQ ID NO: 13, or said amino acid sequences encoded by said nucleic acid sequences encoding the amino acid sequences lacking the signal sequences;

(c) a heavy chain VDJ region amino acid sequence encoded by the nucleic acid sequence of SEQ ID NO: 24, and a light chain VJ region amino acid sequence encoded by the nucleic acid sequence of SEQ ID NO: 14, or said amino acid sequences encoded by said nucleic acid sequences encoding the amino acid sequences lacking the signal sequences;

(d) a heavy chain VDJ region amino acid sequence encoded by the nucleic acid sequence of SEQ ID NO: 26, and a light chain VJ region amino acid sequence encoded by the nucleic acid sequence of SEQ ID NO: 16, or said amino acid sequences encoded by said nucleic acid sequences encoding the amino acid sequences lacking the signal sequences;

(e) a heavy chain VDJ region amino acid sequence encoded by the nucleic acid sequence of SEQ ID NO: 32, and a light chain VJ region amino acid sequence encoded by the nucleic acid sequence of SEQ ID NO: 17, or said amino acid sequences encoded by said nucleic acid sequences encoding the amino acid sequences lacking the signal sequences;

(f) a heavy chain VDJ region amino acid sequence encoded by the nucleic acid sequence of SEQ ID NO: 25, and a light chain VJ region amino acid sequence encoded by the nucleic acid sequence of SEQ ID NO: 18, or said amino acid sequences encoded by said nucleic acid sequences encoding the amino acid sequences lacking the signal sequences;

(g) a heavy chain VDJ region amino acid sequence encoded by the nucleic acid sequence of SEQ ID NO: 22, and a light chain VJ region amino acid sequence encoded by the nucleic acid sequence of SEQ ID NO: 19, or said amino acid sequences encoded by said nucleic acid sequences encoding the amino acid sequences lacking the signal sequences;

(h) a heavy chain VDJ region amino acid sequence encoded by the nucleic acid sequence of SEQ ID NO: 23, and a light chain VJ region amino acid sequence encoded by the nucleic acid sequence of SEQ ID NO: 20, or said amino acid sequences encoded by said nucleic acid sequences encoding the amino acid sequences lacking the signal sequences; and

(i) a heavy chain VDJ region amino acid sequence encoded by the nucleic acid sequence of SEQ ID NO: 27, and a light chain VJ region amino acid sequence encoded by the nucleic acid sequence of SEQ ID NO: 21, or said amino acid sequences encoded by said nucleic acid sequences encoding the amino acid sequences lacking the signal sequences.

This invention also provides a nucleic acid molecule (in certain embodiments, said nucleic acid molecule is an isolated nucleic acid molecule) comprising a nucleic acid sequence of: SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20 or SEQ ID NO: 21.

This invention also provides a nucleic acid molecule (in certain embodiments, said nucleic acid molecule is an isolated nucleic acid molecule) comprising a nucleic acid sequence of: SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31 or SEQ ID NO: 32.

This invention also provides a nucleic acid molecule (in certain embodiments, said nucleic acid molecule is an isolated nucleic acid molecule) comprising a nucleic acid sequence that hybridizes under stringent conditions to and that is at least 80% complementary to any of the nucleic acid sequences selected from the group consisting of: SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31 and SEQ ID NO: 32.

This invention also provides a DNA molecule (could be an isolated DNA molecule) encoding an immunoglobulin comprising an amino acid sequence selected from the group consisting of: SEQ ID NO: 47, SEQ ID NO: 48, SEQ ID NO: 49, SEQ ID NO: 50, SEQ ID NO: 51, SEQ ID NO: 52, SEQ ID NO: 53 and SEQ ID NO: 54.

This invention also provides a DNA molecule (could be an isolated DNA molecule) encoding an immunoglobulin comprising an amino acid sequence selected from the group consisting of: SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63 and SEQ ID NO: 64.

This invention also provides a DNA molecule (could be an isolated DNA molecule) encoding an immunoblogulin comprising a heavy chain VDJ region amino acid sequence and light chain VJ region amino acid sequence selected from the group consisting of:

a) a heavy chain VDJ region amino acid sequence of SEQ ID NO: 1, and a light chain VJ region amino acid sequence encoded by the nucleic acid sequence of SEQ ID NO: 8, or said amino acid sequences lacking the signal sequences;

b) a heavy chain VDJ region amino acid sequence of SEQ ID NO: 2, and a light chain VJ region amino acid sequence encoded by the nucleic acid sequence of SEQ ID NO: 9, or said amino acid sequences lacking the signal sequences;

c) a heavy chain VDJ region amino acid sequence of SEQ ID NO: 3, and a light chain VJ region amino acid sequence encoded by the nucleic acid sequence of SEQ ID NO: 10, or said amino acid sequences lacking the signal sequences;

d) a heavy chain VDJ region amino acid sequence of SEQ ID NO: 55, and a light chain VJ region amino acid sequence encoded by the nucleic acid sequence of SEQ ID NO: 6, or said amino acid sequences lacking the signal sequences;

e) a heavy chain VDJ region amino acid sequence of SEQ ID NO: 56, and a light chain VJ region amino acid sequence encoded by the nucleic acid sequence of SEQ ID NO: 47, or said amino acid sequences lacking the signal sequences;

f) a heavy chain VDJ region amino acid sequence of SEQ ID NO: 57, and a light chain VJ region amino acid sequence encoded by the nucleic acid sequence of SEQ ID NO: 7, or said amino acid sequences lacking the signal sequences;

g) a heavy chain VDJ region amino acid sequence of SEQ ID NO: 58, and a light chain VJ region amino acid sequence encoded by the nucleic acid sequence of SEQ ID NO: 53, or said amino acid sequences lacking the signal sequences;

h) a heavy chain VDJ region amino acid sequence of SEQ ID NO: 59, and a light chain VJ region amino acid sequence encoded by the nucleic acid sequence of SEQ ID NO: 54, or said amino acid sequences lacking the signal sequences;

i) a heavy chain VDJ region amino acid sequence of SEQ ID NO: 60, and a light chain VJ region amino acid sequence encoded by the nucleic acid sequence of SEQ ID NO: 51, or said amino acid sequences lacking the signal sequences;

j) a heavy chain VDJ region amino acid sequence of SEQ ID NO: 61, and a light chain VJ region amino acid sequence encoded by the nucleic acid sequence of SEQ ID NO: 48, or said amino acid sequences lacking the signal sequences;

k) a heavy chain VDJ region amino acid sequence of SEQ ID NO: 62, and a light chain VJ region amino acid sequence encoded by the nucleic acid sequence of SEQ ID NO: 52, or said amino acid sequences lacking the signal sequences;

1) a heavy chain VDJ region amino acid sequence of SEQ ID NO: 63 and a light chain VJ region amino acid sequence encoded by the nucleic acid sequence of SEQ ID NO: 50, or said amino acid sequences lacking the signal sequences;

and

m) a heavy chain VDJ region amino acid sequence of SEQ ID NO: 64, and a light chain VJ region amino acid sequence encoded by the nucleic acid sequence of SEQ ID NO: 49, or said amino acid sequences lacking the signal sequences.

This invention also provides a nucleic acid molecule (in certain embodiments, said nucleic acid molecule is an isolated nucleic acid molecule) comprising a nucleic acid sequence that hybridizes under stringent conditions to and that is at least 80% complementary to any of the nucleic acid sequences selected from the group consisting of: SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20 and SEQ ID NO: 21.

This invention also provides a nucleic acid molecule (in certain embodiments, said nucleic acid molecule is an isolated nucleic acid molecule) comprising a nucleic acid sequence that is at least 60% homologous to any of the nucleic acid sequence selected from the group consisting of: SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31 and SEQ ID NO: 32.

This invention also provides a nucleic acid molecule (in certain embodiments, said nucleic acid molecule is an isolated nucleic acid molecule) comprising a nucleic acid sequence that is at least 60% homologous to any of the nucleic acid sequence selected from the group consisting of: SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20 and SEQ ID NO: 21.

### Vectors

To express the antibodies, DNAs encoding partial or full-length light and heavy chains, obtained as described above, are inserted into expression vectors such that the genes are operatively linked to transcriptional and translational control sequences. Expression vectors include plasmid, retroviruses, cosmids, YACs, EBV derived episomes, and the like. The antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector. In a preferred embodiment, both genes are inserted into the same expression vector. The antibody genes are inserted into the expression vector by standard methods (e.g., ligation of complementary restriction sites on the antibody gene fragment and vector, or blunt end ligation if no restriction sites are present).

A convenient vector is one that encodes a functionally complete human CH or CL immunoglobulin sequence, with appropriate restriction sites engineered so that any VH or VL sequence can be easily inserted and expressed, as described above. In such vectors, splicing usually occurs between the splice donor site in the inserted J region and the splice acceptor site preceding the human C region, and also at the splice regions that occur within the human CH exons. Polyadenylation and transcription termination occur at native chromosomal sites downstream of the coding regions. The recombinant expression vector can also encode a signal peptide that facilitates secretion of the antibody chain from a host cell. The antibody chain gene may be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors of the invention carry regulatory sequences that control the expression of the antibody chain genes in a host cell. It will be appreciated by those skilled in the art that the design of the expression vector, including the selection of regulatory sequences may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from retroviral LTRs, cytomegalovirus (CMV) (such as the CMV promoter/enhancer), Simian Virus 40 (SV40) (such as the SV40 promoter/enhancer), adenovirus, (e.g., the adenovirus major late promoter (AdMLP)), polyoma and strong mammalian promoters such as native immunoglobulin and actin promoters. For further description of viral regulatory elements, and sequences thereof, see, e.g., U.S. Pat. No. 5,168,062 by Stinski, U.S. Pat. No. 4,510,245 by Bell et al. and U.S. Pat. No. 4,968,615 by Schaffner et al.

In addition to the antibody chain genes and regulatory sequences, the recombinant expression vectors of the invention may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr-host cells with methotrexate selection/amplification) and the neo gene (for G418 selection).

### Non-hybridoma Host Cells and Methods of Recombinantly Producing Protein

Nucleic acid molecules encoding antibodies, or antigen-binding portions thereof, that bind one or more mitochondrial antigens bound by a human autoantibody found in patients with PBC and vectors comprising these nucleic acid molecules can be used for transformation of a suitable mammalian host cell. Transformation can be by any known method for introducing polynucleotides into a host cell. Methods for introduction of heterologous polynucleotides into mammalian cells are well known in the art and include dextran-mediated transfection, calcium phosphate precipitation, polybrene-mediated transfection, protoplast fusion, electroporation, encapsulation of the polynucleotide(s) in liposomes, and direct microinjection of the DNA into nuclei. In addition, nucleic acid molecules may be introduced into mammalian cells by viral vectors. Methods of transforming cells are well known in the art. See, e.g., U.S. Pat. Nos. 4,399,216, 4,912,040, 4,740,461, and 4,959,455 (the disclosures of which are hereby incorporated herein by reference).

Mammalian cell lines available as hosts for expression are well known in the art and include many immortalized cell lines available from the American Type Culture Collection (ATCC). These include, inter alia, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, and a number of other cell lines. Cell lines of particular preference are selected through determining which cell lines have high expression levels. Other cell lines that may be used are insect cell lines, such as Sf9 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Further, expression of antibodies of the invention (or other moieties therefrom) from production cell lines can be enhanced using a number of known techniques. For example, the glutamine synthetase gene expression system (the GS system) is a common approach for enhancing expression under certain conditions. The GS system is discussed in whole or part in connection with European Patent Nos. 0 216 846, 0 256 055, and 0 323 997 and European Patent Application No. 89303964.4.

### Transgenic Animals

Antibodies described herein also can be produced transgenically through the generation of a mammal or plant that is transgenic for genes encoding the immunoglobulin heavy and light chain sequences of the antibody of interest or of an antigen-binding portion thereof and production of the antibody in a recoverable form therefrom. In connection with the transgenic production in mammals, antibodies can be produced in, and recovered from, for example, the milk of lactating mammals such as goats, cows. See, e.g., U.S. Pat. Nos. 5,827,690, 5,756,687, 5,750,172, and 5,741,957.

In another embodiment, the transgenic animals or plants comprise nucleic acid molecules encoding antibodies to one or more mitochondrial antigens bound by a human autoantibody found in patients with PBC. In a preferred embodiment, the transgenic animals or plants comprise nucleic acid molecules encoding heavy and light chains specific for a mitochondrial antigen bound by a human autoantibody found in patients with PBC.

In another embodiment, the transgenic animals or plants comprise nucleic acid molecules encoding a modified antibody such as a single-chain antibody, a chimeric antibody or a humanized antibody. The antibodies to one or more mitochondrial antigens bound by a human autoantibody found in patients with PBC may be made in any transgenic animal or plants. In a preferred embodiment, the non-human animals are, without limitation, mice, rats, sheep, pigs, goats, cattle or horses; and the plants are, without limitation, tobacco, corn, or soy. As will be appreciated, proteins may also be generated in eggs that are transgenic for the genes encoding the proteins, such as chicken eggs, among other things.

### Phage Display Libraries

Human antibodies or antigen-binding portions thereof of the invention in addition to the human antibodies disclosed herein can be isolated by screening recombinant combinatorial antibody libraries, preferably a scFv phage display library, prepared using human VL and VH cDNAs prepared from mRNA derived from human lymphocytes. Methodologies for preparing and screening such libraries are known in the art. There are commercially available kits for generating phage display libraries (e.g., the Pharmacia Recombinant Phage Antibody System, catalog no. 27-9400-01; and the Stratagene SurfZAP™ phage display kit, catalog no. 240612). There are also other methods and reagents that can be used in generating and screening antibody display libraries (see. e.g., Ladner et al. U.S. Pat. No. 5,223,409; Kang et al. PCT Publication No. WO 92/18619; Dower et al. PCT Publication No. WO 91/17271; Winter et al. PCT Publication No. WO 92/20791; Markland et al. PCT Publication No. WO 92/15679; Breitling et al. PCT Publication No. WO 93/01288; McCafferty et al. PCT Publication No. WO 92/01047; Garrard et al. PCT Publication No. WO 92/09690; Fuchs et al. (1991) Bio/Technology 9:1370-1372; Hay et al. (1992) Hum. Antibod. Hybridomas 3:81-85; Huse et al. (1989) Science 246:1275-1281; McCafferty et al., Nature (1990) 348:552-554; Griffiths et al. (1993) EMBO J 12:725-734; Hawkins et al. (1992) J. Mol. Biol. 226:889-896; Clackson et al. (1991) Nature 352:624-628; Gram et al. (1992) Proc. Natl. Acad. Sci. USA 89:3576-3580; Garrad et al. (1991) Bio/Technology 9:1373-1377; Hoogenboom et al. (1991) Nuc Acid Res 19:4133-4137; and Barbas et al. (1991) Proc. Natl. Acad. Sci. USA 88:7978-7982.

In a preferred embodiment, to isolate human antibodies to a mitochondrial antigen bound by a human autoantibody found in patients with PBC with the desired characteristics, a human antibody to a mitochondrial antigen bound by a human autoantibody found in patients with PBC as described herein is first used to select human heavy and light chain sequences having similar binding activity toward a mitochondrial antigen bound by a human autoantibody found in patients with PBC, using the epitope imprinting methods described in Hoogenboom et al., PCT Publication No. WO 93/06213. The antibody libraries used in this method are preferably scFv libraries prepared and screened as described in McCafferty et al., PCT Publication No. WO 92/01047, McCafferty et al., Nature (1990) 348:552-554; and Griffiths et al., (1993) EMBO J 12:725-734. The scFv antibody libraries preferably are screened using a mitochondrial antigen bound by a human autoantibody found in patients with PBC as the antigen, respectively.

Once initial human VL and VH segments are selected, “mix and match” experiments, in which different pairs of the initially selected VL and VH segments are screened for to a mitochondrial antigen bound by a human autoantibody found in patients with PBC binding, are performed to select preferred VL/VH pair combinations. Additionally, to further improve the quality of the antibody, the VL and VH segments of the preferred VL/VH pair(s) can be randomly mutated, preferably within the CDR3 region of VH and/or VL, in a process analogous to the in vivo somatic mutation process responsible for affinity maturation of antibodies during a natural immune response. This in vitro affinity maturation can be accomplished by amplifying VH and VL regions using PCR primers complimentary to the VH CDR3 or VL CDR3, respectively, which primers have been “spiked” with a random mixture of the four nucleotide bases at certain positions such that the resultant PCR products encode VH and VL segments into which random mutations have been introduced into the VH and/or VL CDR3 regions. These randomly mutated VH and VL segments can be rescreened for binding to the antigen.

Following screening and isolation of an antibody of the invention from a recombinant immunoglobulin display library, nucleic acid encoding the selected antibody can be recovered from the display package (e.g., from the phage genome) and subcloned into other expression vectors by standard recombinant DNA techniques. If desired, the nucleic acid can be further manipulated to create other antibody forms of the invention, as described below. To express a recombinant human antibody isolated by screening of a combinatorial library, the DNA encoding the antibody is cloned into a recombinant expression vector and introduced into a mammalian host cells, as described above.

### Class Switching

Another aspect of the instant invention is to provide a mechanism by which the class of an antibody described herein may be switched with another. In one aspect of the invention, a nucleic acid molecule encoding VL or VH is isolated using methods well-known in the art such that it does not include any nucleic acid sequences encoding CL or CH. The nucleic acid molecule encoding VL or VH are then operatively linked to a nucleic acid sequence encoding a CL or CH from a different class of immunoglobulin molecule. This may be achieved using a vector or nucleic acid molecule that comprises a CL or CH chain, as described above. For example, an antibody that was originally IgM may be class switched to an IgG. Further, the class switching may be used to convert one IgG subclass to another, e.g., from IgG1 to IgG2.

### Antibody Derivatives

One may use the nucleic acid molecules described above to generate antibody derivatives using techniques and methods known to one of ordinary skill in the art.

### Humanized Antibodies

As was discussed above in connection with human antibody generation, there are advantages to producing antibodies with reduced immunogenicity. This can be accomplished to some extent using techniques of humanization and display techniques using appropriate libraries. It will be appreciated that murine antibodies or antibodies from other species can be humanized or primatized using techniques well known in the art. See e.g., Winter and Harris Immunol Today 14:43-46 (1993) and Wright et al. Crit. Reviews in Immunol. 12125-168 (1992). The antibody of interest may be engineered by recombinant DNA techniques to substitute the CH1, CH2, CH3, hinge domains, and/or the framework domain with the corresponding human sequence (see WO 92/02190 and U.S. Pat. Nos. 5,530,101, 5,585,089, 5,693,761, 5,693,792, 5,714,350, and 5,777,085).

### Mutated Antibodies

In another embodiment, the nucleic acid molecules, vectors and host cells may be used to make mutated antibodies. The antibodies may be mutated in the variable domains of the heavy and/or light chains to alter a binding property of the antibody. For example, a mutation may be made in one or more of the CDR regions to increase or decrease the K_{d} of the antibody for its antigen, to increase or decrease K_{off}, or to alter the binding specificity of the antibody. Techniques in site-directed mutagenesis are well-known in the art. See, e.g., Sambrook et al. and Ausubel et al., supra. In a preferred embodiment, mutations are made at an amino acid residue that is known to be changed compared to germline in a variable region of an antibody of the present invention. In another embodiment, the nucleic acid molecules are mutated in one or more of the framework regions. A mutation may be made in a framework region or constant domain to increase the half-life of the antibody. See. e.g., U.S. application Ser. No. 09/375,924, filed Aug. 17, 1999, herein incorporated by reference. A mutation in a framework region or constant domain may also be made to alter the immunogenicity of the antibody, to provide a site for covalent or non-covalent binding to another molecule, or to alter such properties as complement fixation. Mutations may be made in each of the framework regions, the constant domain and the variable regions in a single mutated antibody. Alternatively, mutations may be made in only one of the framework regions, the variable regions or the constant domain in a single mutated antibody.

In one embodiment, there are no greater than ten amino acid changes in either the VH or VL regions of the mutated antibody compared to the antibody prior to mutation. In a more preferred embodiment, there is no more than five amino acid changes in either the VH or VL regions of the mutated antibody, more preferably no more than three amino acid changes. In another embodiment, there are no more than fifteen amino acid changes in the constant domains, more preferably, no more than ten amino acid changes, even more preferably, no more than five amino acid changes.

### Fusion Antibodies and Immunoadhesins

In another embodiment, a fusion antibody or immunoadhesin may be made which comprises all or a portion of an antibody described herein linked to another polypeptide. In a preferred embodiment, only the variable regions of the antibody are linked to the polypeptide. In another preferred embodiment, the VH domain of an antibody of the present invention is linked to a first polypeptide, while the VL domain of an antibody of this invention is linked to a second polypeptide that associates with the first polypeptide in a manner in which the VH and VL domains can interact with one another to form an antibody binding site. In another preferred embodiment, the VH domain is separated from the VL domain by a linker such that the VH and VL domains can interact with one another (see below under Single Chain Antibodies). The VH-linker-VL antibody is then linked to the polypeptide of interest. The polypeptide may be a therapeutic agent, such as a toxin, growth factor or other regulatory protein, or may be a diagnostic agent, such as an enzyme that may be easily visualized, such as horseradish peroxidase. In addition, fusion antibodies can be created in which two (or more) single-chain antibodies are linked to one another. This is useful if one wants to create a divalent or polyvalent antibody on a single polypeptide chain, or if one wants to create a bispecific antibody.

### Single Chain Antibodies

To create a single chain antibody (scFv), the VH- and VL-encoding DNA fragments are operatively linked to another fragment encoding a flexible linker, e.g., encoding the amino acid sequence (Gly₄-Ser)₃ (SEQ ID NO: 5), such that the VH and VL sequences can be expressed as a contiguous single-chain protein, with the VL and VH regions joined by the flexible linker (see. e.g., Bird et al. (1988) Science 242:423-426; Huston et al. (1988) Proc. Natl. Acad. Sci. USA 85:5879-5883; McCafferty et al., Nature (1990) 348:552-554). The single chain antibody may be monovalent, if only a single VH and VL are used, bivalent, if two VH and VL are used, or polyvalent, if more than two VH and VL are used.

### Kappabodies, Minibodies, Diabodies and Janusins

In another embodiment, other modified antibodies may be prepared using nucleic acid molecules encoding the antibodies described herein. For instance, “Kappa bodies” (Ill et al., Protein Eng 10: 949-57 (1997)), “Minibodies” (Martin et al., EMBO J 13: 5303-9 (1994)), “Diabodies” (Holliger et al., Proc. Nat. Acad. Sci. USA 90: 6444-6448 (1993)), or “Janusins” (Traunecker et al., EMBO J 10: 3655-3659 (1991) and Traunecker et al. “Janusin: new molecular design for bispecific reagents”Int J Cancer Suppl 7:51-52 (1992)) may be prepared using standard molecular biological techniques following the teachings of the specification.

### Chimeric Antibodies

In another aspect, bispecific antibodies can be generated. In one embodiment, a chimeric antibody can be generated that binds specifically to a mitochondrial antigen bound by a human autoantibody found in patients with PBC through one binding domain and to a second molecule through a second binding domain. The chimeric antibody can be produced through recombinant molecular biological techniques, or may be physically conjugated together. In addition, a single chain antibody containing more than one VH and VL may be generated that binds specifically to a mitochondrial antigen bound by a human autoantibody from patients with PBC and to another molecule. Such bispecific antibodies can be generated using techniques that are well known for example, in connection with (i) and (ii) see. e.g., Fanger et al. Immunol Methods 4: 72-81 (1994) and Wright and Harris, supra. and in connection with (iii) see. e.g., Traunecker et al. Int. J. Cancer (Suppl.) 7: 51-52 (1992). A chimeric antibody, for example, could have the framework regions of one antibody and CDR regions of another antibody.

### Derivatized and Labeled Antibodies

An antibody or antibody portion of the invention can be derivatized or linked to another molecule (e.g., another peptide or protein). In general, the antibodies or portion thereof is derivatized such that the binding to a mitochondrial antigen bound by a human autoantibody found in patients with PBC is not affected adversely by the derivatization or labeling. Accordingly, the antibodies and antibody portions of the invention are intended to include both intact and modified forms of the human antibodies to a mitochondrial antigen bound by a human autoantibody found in patients with PBC described herein. For example, an antibody or antibody portion of the invention can be functionally linked (by chemical coupling, genetic fusion, noncovalent association or otherwise) to one or more other molecular entities, such as another antibody (e.g., a bispecific antibody or a diabody), a detection agent, a cytotoxic agent, a pharmaceutical agent, and/or a protein or peptide that can mediate associate of the antibody or antibody portion with another molecule (such as a streptavidin core region or a polyhistidine tag).

One type of derivatized antibody is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

Another type of derivatized antibody is a labeled antibody. Useful detection agents with which an antibody or antibody portion of the invention may be derivatized include fluorescent compounds, including fluorescein, fluorescein isothiocyanate, rhodamine, 5-dimethylamine-1-napthalenesulfonyl chloride, phycoerythrin, lanthanide phosphors and the like. An antibody may also be labeled with enzymes that are useful for detection, such as horseradish peroxidase, β-galactosidase, luciferase, alkaline phosphatase, glucose oxidase and the like. When an antibody is labeled with a detectable enzyme, it is detected by adding additional reagents that the enzyme uses to produce a reaction product that can be discerned. For example, when the agent horseradish peroxidase is present, the addition of hydrogen peroxide and diaminobenzidine leads to a colored reaction product, which is detectable. An antibody may also be labeled with biotin, and detected through indirect measurement of avidin or streptavidin binding. An antibody may also be labeled with a predetermined polypeptide epitopes recognized by a secondary reporter (e.g., leucine zipper pair sequences, binding sites for secondary antibodies, metal binding domains, epitope tags). In some embodiments, labels are attached by spacer arms of various lengths to reduce potential steric hindrance.

An antibody described herein may also be labeled with a radiolabeled amino acid. The radiolabel may be used for both diagnostic and therapeutic purposes. Examples of labels for polypeptides include, but are not limited to, the following radioisotopes or radionuclides—³H, ¹⁴C, N, ³⁵S, ⁹⁰Y, ⁹⁹Tc, ¹¹¹In, ¹²⁵I, ¹³¹I.

An antibody of the present invention may also be derivatized with a chemical group such as polyethylene glycol (PEG), a methyl or ethyl group, or a carbohydrate group. These groups may be useful to improve the biological characteristics of the antibody, e.g., to increase serum half-life or to increase tissue binding.

### Certain Embodiments of the Antibodies of the Invention

This invention provides isolated human antibodies that bind a mitochondrial antigen bound by a human autoantibody found in patients with primary biliary cirrhosis. Preferably, the antibodies are made by the following methods, said methods are also part of this invention.

Thus, an embodiment of this invention is a method of making an isolated human monoclonal antibody that binds a mitochondrial antigen bound by a human autoantibody found in patients with primary biliary cirrhosis comprising the steps of:

a) providing a non-human transgenic animal that has the ability to make human antibodies and that is preferably deficient in the ability to make said animal's antibodies;

b) immunizing said animal with said mitochondrial antigen;

c) obtaining a hybridoma cell line making a monoclonal antibody to said antigen; and

recovering a human antibody.

In a preferred embodiment, said non-human transgenic animal is a mouse, and more preferably said mouse is a XENOMOUSE® animal. In a preferred embodiment, said mitochondrial antigen (could be, for example, human mitochondrial antigen) is selected from the group consisting of the E2 subunit of the pyruvate dehydrogenase complex, the E2 subunit of the branched-chain 2-oxo- acid dehydrogenase complex, the E2 subunit of the 2-oxoglutarate dehydrogenase complex and E3-binding protein. In another preferred embodiment, said isolated human antibody in the method described above binds to one or more peptide, polypeptide or protein selected from the group consisting of the inner lipoyl domain of the E2 subunit of pyruvate dehydrogenase complex; the E2 subunit of the pyruvate dehydrogenase complex; the E2 subunit of the 2-oxoglutarate dehydrogenase complex; the E2 subunit of the branched-chain 2-oxo-acid dehydrogenase complex, MIT3 and p100; in some embodiments, said peptide, polypeptide or protein is of human origin.

An antibody of this invention may be used as a diagnostic agent. For instance, after a patient with PBC has undergone liver transplant, an antibody of this invention may be used to monitor whether the transplanted liver is expressing a mitochondrial antigen bound by an autoantibody from a patient with PBC, by methods well known in the art, such as immunohistochemistry of biopsy of transplanted liver.

### Class and Subclass of Antibodies

The class and subclass of antibodies of the present invention may be determined by any method known in the art. In general, the class and subclass of an antibody may be determined using antibodies that are specific for a particular class and subclass of antibody. Such antibodies are available commercially. The class and subclass can be determined by ELISA, Western Blot as well as other techniques. Alternatively, the class and subclass may be determined by sequencing all or a portion of the constant domains of the heavy and/or light chains of the antibodies, comparing their amino acid sequences to the known amino acid sequences of various class and subclasses of immunoglobulins, and determining the class and subclass of the antibodies.

In one embodiment of the invention, the antibody is a polyclonal antibody. In another embodiment, the antibody is a monoclonal antibody. The antibody may be an IgG, an IgM, an IgE, an IgA or an IgD molecule. In a preferred embodiment, the antibody is an IgG and is an IgG1, IgG2, IgG3 or IgG4 subtype. In a more preferred embodiment, the antibodies are subclass IgG2.

In another embodiment of this invention, a method is provided of obtaining a potential antagonist of a human antibody that binds to a mitochondrial antigen bound by a human autoantibody found in patients with primary biliary cirrhosis, comprising the step of screening for a potential antagonist in a transgenic non-human animal that produces an antibody of this invention (the animal has been immunized with a PBC autoantigen), wherein said potential antagonist reduces the production of said antibody in said transgenic non-human animal. The level of said antibody may be monitored by any methods well known in the art, such as monitoring the level of the antibody in sera of said animal, by, for example, ELISA or RIA.

In another embodiment of this invention, a method is provided of obtaining a potential antagonist of an isolated human antibody that binds to a mitochondrial antigen bound by a human autoantibody found in patients with primary biliary cirrhosis, comprising the step of screening for a potential antagonist in an in vitro assay, wherein said potential antagonist interrupts the interaction of an antibody of this invention with one or more antigens bound by said antibody. Any suitable in vitro assay may be used. An example of a suitable assay is an ELISA.

An antagonist of this invention may be used to treat a patient with PBC, or to prophylactically treat a subject about to have PBC. Thus, in another embodiment of this invention, a method is provided of treating or preventing primary biliary cirrhosis, comprising the step of administering the antagonist identified by methods of this invention comprising the step of administering a pharmaceutical composition comprising said antagonist to a subject in need thereof.

An anti-idiotype antibody of an antibody of this invention may be made by methods well-known in the art. That antibody may be used to treat a patient with PBC, or to prophylactically treat a subject about to have PBC.

All references cited herein are hereby incorporated by reference.

In order that this invention may be better understood, the following examples are set forth. These examples are for purposes of illustration only and are not to be construed as limiting the scope of the invention in any manner.

### EXAMPLE SECTION

### Human Monoclonal Antibodies to Mitochondrial Antigens Bound by Autoantibodies from Patients with PBC

Characterization of the thirteen human mabs described in the Examples below showed that they have a similar Ig gene usage to that found in autoantibodies from patients with PBC. This finding has great implications to identifying the causative agent of PBC. See Fukushima N et al., Int. Immunol.7:1047-1055 (1995); Pascual V et al., J. Immunol. 152:2577-2585 (1994) and Thomson R K et al., J. Hepatology 28:582-594 (1998).

Ig gene analysis of these thirteen human mabs showed that seven used the V_{H}3 repertoire and four used the V_{H}4 repertoire. Three of the thirteen antibodies used the same Ig VH3-21 found in human autoantibodies from patients with PBC. The CDRs of these autoantibodies were slightly mutated when compared to the sequences present in Ig germline genes.

These data suggest that in this unique model system the XENOMOUSE® animals recapitulate the development of the specificities found in the human patient and affirms that autoantibodies to PDC-E2 are produced by a restricted antigen driven clonal selection.

### Example 1

### Human Monoclonal Antibodies Obtained from Xenomouse® Mice

To generate antibodies to mitochondrial antigens implicated in PBC, we immunized XENOMOUSE® animals with either PDC-E2 or recombinant PDC-E2 (“rPDC-E2”).

Antigens

The PDC-E2 fraction of beef heart mitochondria (“BHM”) was isolated by polyethylene glycol precipitation (Yang D et al., J Biol Chem 272:6361-6369 (1997)) (“native PDC-E2”).

We prepared recombinant (r) PDC-E2, full-length and rPDC-E2-inner lipoyl domain (rPDC-E2-ILD), rOGDC-E2, rBCOADC-E2, rE3BP, and r-MIT3 (TRIPLE HYBRID) as described previously. See Surh C. et al., J. Immunol. 144:3367-3374 (1990); Leung P. et al., Hepatoloqy 22:505-513 (1995), Moteki S. et al., Hepatoloqy 24:97-103 (1996) and U.S. Pat. No. 6,111,071, issued Aug. 29, 2000, U.S. Pat. No. 5,196,319, issued Mar. 23, 1993 and U.S. Pat. No. 5,891,436, issued Apr. 9, 1999, and WO 8804689, published on Jun. 30, 1988, the disclosures of all of which are hereby incorporated by reference herein. Keyhole limpet hemocyanin (KLH) was used as a control antigen.

The antigens described in this section were used in the Examples below.

Immunization of XENOMOUSE® Mice

We maintained XENOMOUSE® mice in a SPF (specific pathogen free) and full-barrier configured animal facility. We immunized XENOMOUSE® mice at 8-10 weeks old were immunized by foot-pad injection with 25 μg of native PDC-E2 (bovine), or KLH as control, emulsified in Ribi adjuvant (Ribi immunochem, MT), twice a week for 4 weeks, or by injection at base of tail with 50 μg of rPDC-E2 (human) emulsified in Freund's complete adjuvant (Sigma, St. Louis, Mo.) followed by 10 μg of antigen in Freund's incomplete adjuvant (Sigma) every two weeks. Control XENOMOUSE® mice received the same dose regimen of KLH. We collected blood and the presence of serum antibodies to mitochondrial antigens bound by autoantibodies from patients with PBC in sera was evaluated by ELISA. At 8-12 weeks after the initial immunization, XENOMOUSE® mice that were positive for antibodies to mitochondrial antigens bound by autoantibodies from patients with PBC were boosted with 25 μg of soluble antigen in PBS 4 days before fusion.

Human Monoclonal Antibody Production and Purification

We removed the inguinal and popliteal lymph nodes and spleen from immunized XENOMOUSE® mice. We carried out the fusion of lymphocytes and splenocytes and selection of hybridomas were carried out as described (Mendez M et al., Nat Genet 15:146-156 (1997)).

ELISA

We identified hybridomas positive for mitochondrial antigens bound by autoantibodies from patients with PBC by ELISA using native and recombinant mitochondrial proteins as antigens. We prepared recombinant (r) PDC-E2, full-length and rPDC-E2-inner lipoyl domain (rPDC-E2-ILD), rOGDC-E2, rBCOADC-E2, rE3BP, and r-MIT3 (TRIPLE HYBRID) as described previously. See Surh C. et al., J. Immunol. 144:3367-3374 (1990); Leung P. et al., Hepatology 22:505-513 (1995), Moteki S. et al., Hepatology 24:97-103 (1996) and U.S. Pat. No. 6,111,071, issued Aug. 29, 2000, U.S. Pat. No. 5,196,319, issued Mar. 23, 1993 and U.S. Pat. No. 5,891,436, issued Apr. 9, 1999, and WO 8804689, published on Jun. 30, 1988, the disclosures of all of which are hereby incorporated by reference herein. Keyhole limpet hemocyanin (KLH) was used as a control antigen.

We coated microtiter plates with native proteins, purified recombinant proteins, and peptide-KLH (control) at 10 μg/ml in carbonate buffer (pH 9.6) overnight at 4° C. After washing three times with 0.05% Tween-20 in PBS (PBS/Tween), the plates were blocked with 3% dry milk powder in PBS for 1 hr at room temperature (RT). We added optimal dilutions of sera or supernatant from each well of the hybridoma containing culture plates into individual wells and incubated for 1 hr at RT. After washing, we incubated the plates with 0.1 ml horseradish peroxidase (HRP) conjugated goat anti-human immunoglobulin (Biosource, Camarillo, Calif.), HRP conjugated anti-human IgG2 (Southern Biotech), or HRP-conjugated anti-human IgM (Southern Biotech) for 1 hr. After washing, we determined reactivity by the addition of 100μl of 40 mM 2,2′-azinobis (3-ethylbenzthiazoline-6-sulfonic acid) in 0.05 M citric acid buffer, pH 5, containing 0.05 M H₂O₂. We stopped the reaction after 15 min by the addition of 100 μl of 5% SDS.

We purified the antibodies from culture media of positive hybridomas using protein-A or protein-L affinity chromatography kit (Pierce, Rockford, Ill.).

Immunoblotting

Briefly, a sample was resuspended in 250 μl of sample buffer (125 mM Tris-HCl (pH 6.8) containing 4% SDS, 20% glycerol, and 5% 2-ME), boiled for 5 min, and resolved by SDS-PAGE using 1.5 mm-thick slab gels with a 4.75% stacking gel and a 10% separating gel. Separated proteins were transferred electrophoretically to nitrocellulose filters (Micron Separations, Westboro, Mass.). After transfer, nitrocellulose filters were blocked in 3% milk powder in PBS for 1 hr at RT and probed by incubation for 1 hr with optimally titered XENOMOUSE® sera or human mAbs diluted at 1:5. PBC, normal human sera and normal mouse sera were used throughout as controls. After washing with PBS/Tween, the strips were incubated for an additional hour with goat anti-human polyvalent Ig (Biosource) (1:2000). The strips were washed and visualized with enhanced chemiluminescent substrate (Pierce, Rockford, Ill.). A representative immunoblot of two IgG2 and two IgM isotype human mAbs obtained from XENOMOUSE® animals is shown in FIG. 1.

Epitope Mapping

To locate the epitope bound by the human mAbs described in Example 1 (obtained from XENOMOUSE® animals), recombinant peptides consisting of the outer lipoyl domain (OLD), inner lipoyl domain (ILD), E1/E3 binding domain, and catalytic domain of human PDC-E2 were prepared (Surh C. et al., J. Immunol. 144: 3367-3374 (1990)). We determined the specificity of PDC-E2 specific human mAbs against a series of overlapping PDC-E2 recombinant peptides by ELISA as described (Leung PSC et al., Semin. Liver Dis. 17:61-69 (1997)).

Specificities of Human mAbs

We obtained a total of thirteen stable anti-mitochondrial antigen specific human mAbs (HmAbs) from XENOMOUSE® animals (FIG. 6). Four of the thirteen antibodies were IgG2 and nine were IgM antibodies (FIG. 6). When compared to the response in patients, the relative absence of IgMs in the patients may reflect a long period of disease development from which reagents are unavailable, during which class switching in the patient may occur.

Twelve of the thirteen human mAbs obtained from XENOMOUSE® animals reacted with one or more members of the 2-oxo-acid dehydrogenase family (FIG. 6). Monoclonal antibodies 4D11 and 3B4 were obtained from XENOMOUSE® mice immunized with r-PDC-E2 and the rest were obtained from XENOMOUSE® mice immunized with native PDC-E2. These human anti-mitochondrial antigens mAbs obtained from XENOMOUSE® animals have a number of features in common with the autoantibodies that occur in patients with PBC. Six of the thirteen human mabs reacted with PDC-E2 only: four of these six clones recognized the inner lipoyl domain of PDC-E2; the remaining two (2C8 and 4C2) reacted with full length PDC-E2, but not with any of the PDC-E2 individual domains (i.e., the outer lipoyl, inner lipoyl, E1/E3 binding and the catalytic domains)(FIG. 6). Four of the thirteen human mAbs reacted with both PDC-E2 and OGDC-E2. This is similar to data from murine monoclonal antibody to PDC-E2 (Migliaccio C et al., J Immunol 161:5157-5163 (1998)). These four mAbs also bound the inner lipoyl domain of PDC-E2 and MIT3 (a synthetic hybrid of the autoepitopes of the three E2 enzymes (i.e., MIT3 co-expresses the three immunodominant lipoyl domains of PDC-E2, BCOADC-E2 and OGDC-E2)(FIG. 6)). The cross-reactivity between PDC-E2 and other mitochondrial antigens may be the result of cross-reactivity between these lipoic acid containing epitopes. For the same reason, the human mAbs raised to purified native PDC-E2 are seeing cross-reactive epitopes in the other proteins. Not all of these epitopes necessarily are in association with lipoic acid as there are relatively few lipoic acid containing proteins in the cell, and none has been reported to be found in the nucleus.

This ability of the human mAbs (as observed by the monoclonal antibodies obtained from XENOMOUSE® animals) to make a cross-reactive response to other antigens, when immunized with only PDC-E2, has implications for the etiology of this response. In the past, the multiple targets for human autoantibodies in PBC were viewed to suggest a complex immunizing event, such as an event that might occur with a micro-organism. The results here are compatible with a simpler explanation, such as a single molecule, either a normal protein or one modified by environmental agents, that induces responses to multiple targets. As tolerance is broken, and these multiple targets are further responded to, a complex series of antibody responses may arise by a process of determinant spreading.

Most surprisingly, two of the human mAbs did not bind to PDC-E2. One human mAb reacted with OGDC-E2 only and one human mAb of reacted with BCOADC-E2 only (FIG. 6). The OGDC specific antibody and the BCOADC specific antibody recognized the lipoyl domains of their respective target proteins, showing a similar epitopic region to that seen in human antibodies (Van de Water J et al., Journal of Immunology 141:2321-2324 (1988)).

One human mAb (3E7) of the thirteen reacted with a 100kD mitochondrial protein, but did not react with the inner lipoyl domain of PDC-E2 or with MIT3 (FIG. 6).

We sequenced the variable regions of the heavy and/or light chains of the human mAbs produced from XENOMOUSE® animals.

Almost all of the nucleic acid sequences of the VDJ regions of the heavy chains and the VJ regions of the light chains of these thirteen human mabs that bind one or more mitochondrial antigens bound by autoantibody from patients with PBC (these thirteen human mAbs are obtained from XENOMOUSE® animals) are provided in Table 1.

TABLE 1

3D8 kappa light chain VJ region

| | | | | | |
|---|---|---|---|---|---|
| TCCTCCCTGT | CTGCATCTGT | TGGAGACAGA | GTCACCATCA | CTTGCCGGGC | (SEQ ID NO: 12) |
| AAGTCAGGGC | ATTAGAAATG | ATTTAGGCTG | GTATCAGCAG | AAACCAGGGA | |
| AAGCCCCTAA | GCGCCTGATC | TATGCTGCAT | CCAGTTTGCA | AAGTGGGGTC | |
| CCATCAAGGT | TCAGCGGCAG | TGGATCTGGG | ACAGAATTCA | CTCTCACAAT | |
| CAGCAGCCTG | CAGCCTGAAG | ATTTTGCAAC | TTATTATTGT | CTACAGTATA | |
| ATAGTTACCC | ATTCATTTTC | GGCCCTGGGA | CCAAAGTGGA | TATCAAACGA | |
| ACTGTGGCTG | CACCATCTGT | CTTCATCTTC | CCGCCATCTG | ATGAGCAGTT | |
| GAAATCTGGA | ACTGCCTCTG | TTGTGTGCCT | GCTGAATAAC | TTCTATCCCA | |
| GAGAGGCCAA | AGTACAGTGG | AAGGTGGATA | ACGCCCTCCA | A | |

4B10 kappa light chain VJ region

| | | | | | |
|---|---|---|---|---|---|
| CCTGTCTGCA | TCTGTAGGAG | ACAGAGTCAC | CATCACTTGC | CGGGCAAGTC | (SEQ ID NO: 13) |
| AGGGCATTAG | AAATGATTTA | GGCTCGTATC | AGCAGAAACC | AGGGAAAGCC | |
| CCTAAGCGCC | TGATCTATGC | TGCATCCAGT | TTGCAAAGTG | GGGTCCCATC | |
| AAGGTTCAGC | GGCAGTGGAT | CTGGGACAGA | ATTCACTCTC | ACAATCAGCA | |
| GCCTGCAGCC | TGAAGATTTT | GCAACTTATT | ACTGTCTACA | GTATAATAGT | |
| TACCCATTCA | CTTTCGGCCC | TGGGACCAAA | GTGGATATCA | AACGAACTGT | |
| GGCTGCACCA | TCTGTCTTCA | TCTTCCCGCC | ATCTGATGAG | CAGTTGAAAT | |
| CTGGAACTGC | CTCTGTTGTG | TGCCTGCTGA | ATAACTTCTA | TCCCAGAGAG | |
| GCCAAAGTAC | AGTGGAAGGT | GGATAACGCC | CTCCA | | |

2E11 kappa light chain VJ region

| | | | | | |
|---|---|---|---|---|---|
| GCCCGTCACC | CTTGGACAGC | CGGCCTCCAT | CTCCTGCAGG | TCTAGTCAAA | (SEQ ID NO: 14) |
| GCCTCGTATA | CAGTGATGGA | AACACCTTCT | TGAATTGGTT | TCAGCAGAGG | |
| CCAGGCCAAT | CTCCAAGGCG | CCTAATTTAT | AAGGTTTCTA | ACTGGGACTC | |
| TGGGGTCCCA | GACAGATTCA | GCGGCAGTGG | GTCAGGCACT | GATTTCACAC | |
| TGAAAATCAG | CAGGGTGGAG | GCTGAGGATG | TTGGGGTTTA | TTACTGCATG | |
| CAAGGTACAC | ACTGGCCTAT | CACCTTCGGC | CAAGGGACAC | GACTGGAGAT | |
| TAAACGAACT | GTGGCTGCAC | CATCTGTCTT | CATCTTCCCG | CCATCTGATG | |
| AGCAGTTGAA | ATCTGGAACT | GCCTCTGTTG | TGTGCCTGCT | GAATAACTTC | |
| TATCCCAGAG | AGGGC | | | | |

2E6 kappa light chain VJ region

| | | | | | |
|---|---|---|---|---|---|
| TGTAGGAGAC | AGAGTCACCA | TCACTTGCCG | GGCGAGTCAG | GGCATTAGCA | (SEQ ID NO: 15) |
| ATTATTTAGC | CTGGTATCAG | CAGAAACCAG | GGAAAGTTCC | TAAGCTCCTG | |
| ATCTATGCTG | CATCCACTTT | GCAATCAGGG | GTCCCATCTC | GGTTCAGTGG | |
| CAGTGGATCT | GGGACAGATT | TCACTCTCAC | CATCAGCAGC | CTGCAGCCTG | |
| AAGATGTTGC | AACTTATTAC | TGTCAAAAGT | ATAACAGTGC | CCCATTCACT | |
| TTCGGCCCTG | GGACCAAAGT | GGATATCAAA | CGAACTGTGG | CTGCACCATC | |
| TGTCTTCATC | TTCCCGCCAT | CTGATGAGCA | GTTGAAATCT | GGAACTGCCT | |
| CTGTTGTGTG | CCTGCTGAAT | AACTTCTATA | ACAGAGAGGN | G | |

3B4 kappa light chain VJ region

| | | | | | |
|---|---|---|---|---|---|
| GTAGGAGACA | GAGTCACCAT | CACTTGCCGG | GCAAGTCAGG | GCATTAGAAA | (SEQ ID NO: 16) |
| TGATTTAGGC | TGGTATCAGC | AGAAACCAGG | GAAAGCCCCT | AAGCGCCTGA | |
| TCTATGCTGC | ATCCAGTTTG | CAAAGTGGGG | TCCCATCAAG | GTTCAGCGGC | |
| AGTGGATCTG | GGACAGAATT | CACTCTCACA | ATCAGCAGCC | TGCAGCCTGA | |
| AGATTTTGCA | ACTTATTACT | GTCTACAGCA | TAATAGTTAC | CCTCGGACGT | |
| TCGGCCAAGG | GACCAAGGTG | GAAATCAAAC | GAACTGTGGC | TGCACCATCT | |
| GTCTTCATCT | TCCCGCCATC | TGATGAGCAG | TTGAAATCTG | GAACTGCCTC | |
| TGTTGTGTGC | CTGCTGAATA | ACTTCTATAC | CCAGAGAGG | | |

3E7 kappa light chain VJ region

| | | | | | |
|---|---|---|---|---|---|
| ATCTCCTGCA | GGTCTAGTCA | GAGCCTCCTG | CATAGTAATG | GATACAACTA | (SEQ ID NO: 17) |
| TTTGGATTGG | TACCTGCAGA | AGCCAGGGCA | GTCTCCACAG | CTCCTGATCT | |
| ATTTGGGTTC | TAATCGGGCC | TCCGGGGTCC | CTGACAGGTT | CAGTGGCAGT | |
| GGATCAGGCA | CAGATTTTAC | ACTGAAAATC | AGCAGAGTGG | AGGCTGAGGA | |
| TGTTGGGGTT | TATTACTGCA | TGCAAGCTCT | ACAAACTCCG | TGGACGTTCG | |
| GCCAAGGGAC | CAAGGTGGAA | ATCAAACGAA | CTGTGGCTGC | ACCATCTGTC | |
| TTCATCTTCC | CGCCATCTGA | TGAGCAGTTG | AAATCTGGAA | CTGCCTCTGT | |
| TGTGTGCCTG | CTGAATAACT | TCTATANCAN | AGAGGAGGG | | |

4C2 kappa light chain VJ region

| | | | | | |
|---|---|---|---|---|---|
| TCTGGGCGAG | AGGGCCACCA | TCAACTGCAA | GTCCAGCCAG | AGTGTTTTAT | (SEQ ID NO: 18) |
| ACAGCTCCAA | CAATAAGAAC | TACTTAGCTT | GGTACCAGCA | GAAACCAGGA | |
| CAGCCTCCTA | AGTTGCTCAT | TTACTGGGCA | TCTACCCGGG | AATCCGGGGT | |
| CCCTGACCGA | TTCAGTGGCA | GCGGGTCTGG | GACAGATTTC | ACTCTCACCA | |
| TCAGCAGCCT | GCAGGCTGAA | GATGTGGCAG | TTTATTACTG | TCAGCAATAT | |
| TATAGTACTC | CGTGGACGTT | CGGCCAAGGG | ACCAAGGTGG | AAATCAAACG | |
| AACTGTGGCT | GCACCATCTG | TCTTCATCTT | CCCGCCATCT | GATGAGCAGT | |
| TGAAATCTGG | AACTGCCTCT | GTTGTGTGCC | TGCTGAATAA | CTTCTATCCC | |
| AGAGAGGGGA | TAG | | | | |

4D11 kappa light chain VJ region

| | | | | | |
|---|---|---|---|---|---|
| AGAGTCACCA | TCACTTGTCG | GGCGAGTCGG | GGTATTAGCA | GCTGGTTAGC | (SEQ ID NO: 19) |
| CTGGTATCAG | CAGAAACCAG | GGAAAGCCCC | TAAGCTCCTG | ATCTTTGCTG | |
| CATCCAGTTT | GCAAAGTGGG | GTCCCATCAA | GGTTCAGCGG | CAGTGGATCT | |
| GGGACAGATT | TCACTCTCAC | CATCAGCAGC | CTGCAGCCTG | AAGATTTTGC | |
| AACTTACTTT | TGTCAACAGG | CTAACAGTTT | CCCATTCACT | TTCGGCCCTG | |
| GGACCAAAGT | GGATATCAAA | CGAACTGTGG | CTGCACCATC | TGTCTTCATC | |
| TTCCCGCCAT | CTGATGAGCA | GTTGAAATCT | GGAACTGCCT | CTGTTGTGTG | |
| CCTGCTGAAT | AACTTCTATC | CCAGAGAGGG | CAT | | |

1C7 kappa light chain VJ region

| | | | | | |
|---|---|---|---|---|---|
| GTGTCTCTGG | GCGAGAGGGC | CACCATCAAC | TGCAAGTCCA | GCCAGAGTGT | (SEQ ID NO: 20) |
| TTTATACAGC | TCCAACAATA | AGAACTACTT | AGCTTGGTAC | CAGCAGAAAC | |
| CAGGACAGCC | TCCTAAGCTG | CTCATTTACT | GGGCATCTAC | CCGGGAATCC | |
| GGGGTCCCTG | ACCGATTCAG | TGGCAGCGGG | TCTGGGACAG | ATTTCACTCT | |
| CACCATCAGC | AGCCTGCAGG | CTGAAGATGT | GGCAGTTTAT | TACTGTCAGC | |
| AATATTATAG | TACTCCTCGG | ACGTTCGGCC | AAGGGACCAA | GGTGGAAATC | |
| AAACGAACTG | TGGCTGCACC | ATCTGTCTTC | ATCTTCCCGC | CATCTGATGA | |
| GCAGTTGAAA | TCTGGAACTG | CCTCTGTTGT | GTGCCTGCTG | AATAACTTCT | |
| ATAACAGAGA | GGGATNCCAA | AGAGTC | | | |

1F8 kappa light chain VJ region

| | | | | | |
|---|---|---|---|---|---|
| CCTGTCTGCA | TCTGTAGGAG | ACAGAGTCAC | CATCACTTGC | CGGGCAAGTC | (SEQ ID NO: 21) |
| AGGGCATTAG | AAATGATTTA | GGCTGGTATC | AGCAGAAACC | AGGGAAAGCC | |
| CCTAAGCGCC | TGATCTATGC | TGCATCCAGT | TTGCAAAGTG | GGGTCCCATC | |
| AAGGTTCAGC | GGCAGTGGAT | CTGGGACAGA | ATTCACTCTC | ACAATCAGCA | |
| GCCTGCAGCC | TGAAGATTTT | GCAACTTATT | ACTGTCTACA | GTATAATAGT | |
| TACCCATTCA | CTTTCGGCCC | TGGGACCAAA | GTGGATATCA | AACGAACTGT | |
| GGCTGCACCA | TCTGTCTTCA | TCTTCCCGCC | ATCTGATGAG | CAGTTGAAAT | |
| CTGGAACTGC | CTCTGTTGTG | TGCCTGCTGA | ATAACTTCTA | TCCCAGAGAG | |
| GCCAAAGTAC | AGTGGAAGGT | GGATAACGCC | CTCCA | | |

4D11 heavy chain VDJ region

| | | | | | |
|---|---|---|---|---|---|
| GGGGAGGCGT | GGTCCAGCCT | GGGAGGTCCC | TGAGACTCTC | CTGTGCAGCG | (SEQ ID NO: 22) |
| TCTGGATTCA | CCTTCAGTAG | CTATGGCATG | CACTGGGTCC | GCCAGGCTCC | |
| AGGCAAGGGG | CTGGAGTGGG | TGGCAGTTAT | ATGGTATGAT | GGAAGTAATA | |
| AATACTATGC | AGACTCCGTG | AAGGGCCGAT | TCACCATCTC | CAGAGACAAT | |
| TCCAAGAACA | CGCTGTATCT | GCAAATGAAC | AGCCTGAGAG | CCGAGGACAC | |
| GGCTGTGTAT | TACTGTGCGA | GATCTCCTGC | GGACTGGTAC | TTCGATCTCT | |
| GGGGCCGTGG | CACCCTGGTC | ACTGTCTCCT | CAGCCTCCAC | CAAGGGCCCA | |
| TCGGTCTTCC | CCCTGGCGCC | CTGCTCCAGG | AGCACCTCCG | AGAGCACAGC | |
| GGCCCTGGGC | TGCCTGGTCA | AGGANTACTT | CCCCGAACCG | GTGACG | |

1C7 heavy chain VDJ region

| | | | | | |
|---|---|---|---|---|---|
| CCAGGACTGG | TGAAGCCCTC | GCAGACCCTC | TCACTCACCT | GTGCCATCTC | (SEQ ID NO: 23) |
| CGGGGACAGT | GTCTCTAGCA | ACAGTGCTGC | TTGGAACTGG | ATCAGGCAGT | |
| CCCCATCGAG | AGGCCTTGAG | TGGCTGGGAA | GGACATACTA | CAGGTCCAAG | |
| TGGTATAATG | ATTATGCAGT | ATCTGTGAAA | AGTCGAATAA | CCATCAACCC | |
| AGACACATCC | AAGAACCAGT | TCTCCCTGCA | GCTGAACTCT | GTGACTCCCG | |
| AGGACACGGC | TGTGTATTAC | TGTGCAAGAG | GGTATAGCAG | TGGCTGGTAC | |
| TTTGACTACT | GGGGCCAGGG | AACCCTGGTC | ACCGTCTCCT | CAGGGAGTGC | |
| ATCCGCCCCA | ACCCTTTTCC | CCCTCGTCTC | CTGTGAGAAT | TCCCCGTCGG | |
| ATACGAGCAG | CGTGGCCGTT | GGCTG | | | |

2E11 heavy chain VDJ region

| | | | | | |
|---|---|---|---|---|---|
| GATCCGGCAG | TCCCCAGGGA | AGGGACTGGA | GTGGATTGGA | CACATCTATT | (SEQ ID NO: 24) |
| ACAGTGGGAA | CACCAATTAT | AACCCCTCCC | TCAAGAGTCG | ACTCACCATA | |
| TCAATTGACA | NGTCCAAGAC | TCAGTTCTCC | CTGAAGCTGA | GTTCTGTGAC | |
| CGCTGCGGAC | ACGGCCATTT | ATTACTGTGT | GCNAGATCGA | GTGACTGGTG | |
| CTTTTGATAT | CTGGGGCCAA | GGGACAATGG | TCACCGTCTC | TTCAGCCTCC | |
| ACCAAGGGCC | CATCGGTCTT | CCCCCTGGCG | CCCTGCTCCA | GGAGCACCTC | |
| CGAGAGCACA | GCGGCCCTGG | GCTGCCTGGT | CAAGGACTAC | TTCCCCGAAC | |
| CGGTGACGGT | GTCGTGGAAC | TCAGGCGCTC | TGACCAGCGG | | |

4C2 heavy chain VDJ region

| | | | | | |
|---|---|---|---|---|---|
| TCTCTGGTGG | CTCCGTCAGC | AGTGGTGATT | ACTACTGGAC | CTGGATCCGG | (SEQ ID NO: 25) |
| CAGTCCCCAG | GGAAGGGACT | GGAGTGGATT | GGACACATCT | ATTACAGTGG | |
| GAACACCAAT | TATAACCCCT | CCCTCAAGAG | TCGACTCACC | ATATCAATTG | |
| ACACGTCCAA | GACTCAGTTC | TCCCTGAAGC | TGAGTTCTGT | GACCGCTGCG | |
| GACACGGCCA | TTTATTACTG | TGTGCGAGAT | CGAGTGACTG | GTGCTTTTGA | |
| TATCTGGGGC | CAAGGGACAA | TGGTCACCGT | CTCTTCAGCC | TCCACCAAGG | |
| GCCCATCGGT | CTTCCCCCTG | GCGCCCTGCT | CCAGGAGCAC | CTCCGAGAGC | |
| ACAGCGGCCC | TGGGCTGCCT | GGTCAAGG | | | |

3B4 heavy chain VDJ region

| | | | | | |
|---|---|---|---|---|---|
| GATCCGGCAG | TCCCCAGGGA | AGGGACTGGA | GTGGATTGGA | CACATCTATT | (SEQ ID NO: 26) |
| ACAGTGGGAA | CACCAATTAT | AACCCCTCCC | TCAAGAGTCG | ACTCACCATA | |
| TCAATTGACA | CGTCCAAGAC | TCAGTTCTCC | CTGAAGCTGA | GTTCTGTGAC | |
| CGCTGCGGAC | ACGGCCATTT | ATTACTGTGT | GCGAGATCGA | GTGACTGGTG | |
| CTTTTGATAT | CTGGGGCCAA | GGGACAATGG | TCACCGTCTC | TTCAGCCTCC | |
| ACCAAGGGCC | CATCGGTCTT | CCCCCTGGCG | CCCTGCTCCA | GGAGCACCTC | |
| CGAGAGCACA | GCGGCCCTGG | GCTGCCTGGT | CAAGGACTAC | TTCCCCGAAC | |
| CGGTGACGGT | GTCGTG | | | | |

1F8 heavy chain VDJ region

| | | | | | |
|---|---|---|---|---|---|
| GGGAGGCCTG | GTCAAGCCTG | GGGGGTCCCT | GAGACTCTCC | TGTGCAGCCT | (SEQ ID NO: 27) |
| CTGGATTCAC | CTTCAGTAGC | TATAGCATGA | ACTGGGTCCG | CCAGGCTCCA | |
| GGGAAGGGGC | TGGAGTGGGT | CTCATCCATT | AGTAGTAGTA | GTAGTTACAT | |
| ATACTACGCA | GACTCAGTGA | AGGGCCGATT | CACCATCTCC | AGAGACAACG | |
| CCAAGAACTC | ACTGTATCTG | CAAATGAACA | GCCTGAGAGC | CGAGGACACG | |
| GCTGTGTATT | ACTGTGCGAG | CTCCGGGTAT | AGCAGCAGCT | GGTCTGACTA | |
| CTGGGGCCAG | GGAACCCTGG | TCACCGTCTC | CTCAGGGAGT | GCATCCGCCC | |
| CAACCCTTTT | CCCCCTCGTC | TCCTGTGAGA | ATTCCCCGTC | GGATACGAGC | |
| AGCGTGGCCG | TTGGCTG | | | | |

3D8 heavy chain VDJ region

| | | | | | |
|---|---|---|---|---|---|
| GGGGAGGCCT | GGTCAAGCCT | GGGGGGTCCC | TGAGACTCTC | CTGTGAAGTC | (SEQ ID NO: 28) |
| TCTGGATTCA | CCTTCAGTAG | CTATAGCATG | AACTGGGTCC | GCCAGGCTCC | |
| AGGGAAGGGG | CTGGAGTGGG | TCTCATCCAT | TAGTAGTAGT | AGTAGTTACA | |
| TATACTACGC | AGACTCAGTG | AAGGGCCGAT | TCACCATCTC | CAGAGACAAC | |
| GCCAAGAACT | CACTGTATCT | GCAAATGAAC | AGCCTGAGAG | CCGAGGACAC | |
| GGCTGTGTAT | TACTGTGCGA | TATCAGTGGT | TGACTACTGG | GGCCAGGGAA | |
| CCCTGGTCAC | CGTCTCCTCA | GGGAGTGCAT | CCGCCCCAAC | CCTTTTCCCC | |
| CTCGTCTCCT | GTGAGAATTC | CCCGTCGGAT | ACGAGCAGCG | TGGCCGTTGG | |
| CTG | | | | | |

4B10 heavy chain VDJ region

| | | | | | |
|---|---|---|---|---|---|
| GGGAGGCCTG | GTCAAGCCTG | GGGGGTCCCT | GAGACTCTCC | TGTGCAGCCT | (SEQ ID NO: 29) |
| CTGGATTCAC | CTTCAGTAGC | TATAGCATGA | ACTGGGTCCG | CCAGGCTCCA | |
| GGGAAGGGGC | TGGAGTGGGT | CTCATCCATT | AGTAGTAGTA | GTAGTTACAT | |
| ATACTACGCA | GACTCAGTGA | AGGGCCGATT | CACCATCTCC | AGAGACAACG | |
| CCAAGAACTC | ACTGTATCTG | CAAATGAACA | GCCTGAGAGC | CGAGGACACG | |
| GCTGTGTATT | ACTGTGCGAG | CTCCGGGTAT | AGCAGCAGCT | GGTCTGACTA | |
| CTGGGGCCAG | GGAACCCTGG | TCACCGTCTC | CTCAGGGAGT | GCATCCGCCC | |
| CAACCCTTTT | CCCCCTCGTC | TCCTGTGAGA | ATTCCCCGTC | GGATACGAGC | |
| AGCGTGGCCG | TTGGCTG | | | | |

4G6 heavy chain VDJ region

| | | | | | |
|---|---|---|---|---|---|
| CAGGACTGGT | GAAGCCCTCG | CAGACCCTCT | CACTCACCTG | TGCCATCTCC | (SEQ ID NO: 30) |
| GGGGACAGTG | TCTCTAGCAA | CAGTGCTGCT | TGGAACTGGA | TCAGGCAGTC | |
| CCCATCGAGA | GGCCTTGAGT | GGCTGGGAAG | GACATACTAC | AGGTCCAAGT | |
| GGTATAATGA | TTCTGCAGTA | TCTGTGAAAA | GTCGAATAAC | CATCAACCCA | |
| GACACATCCA | AGAACCAGTT | CTCCCTGCAG | CTGAACTCTG | TGACTCCCGA | |
| GGACACGGCT | GTGTATTACT | GTGCAAGCTG | GATTGCAGCA | GCTGGTTTTG | |
| ACTCCTGGGG | CCAGGGAACC | CTGGTCACCG | TCTCCTCAGG | GAGTGCATCC | |
| GCCCCAACCC | TTTTCCCCCT | CGTCTCCTGT | GAGAATTCCC | CGTCGGATAC | |
| GAGCAGCGTG | GCCGTTGGCT | G | | | |

2G8 heavy chain VDJ region

| | | | | | |
|---|---|---|---|---|---|
| GTCCAGGACT | GGTGAAGCCC | TCGCAGACCC | TCTCACTCAC | CTGTGCCATC | (SEQ ID NO: 31) |
| TCCGGGGACA | GTGTCTCTAG | CAACAGTGCT | GCTTGGAACT | GGATCAGGCA | |
| GTCCCCATCG | AGAGGCCTTG | AGTGGCTGGG | AAGGACATAC | TACAGGTCCA | |
| AGTGGTATAA | TGATTATACA | GTATCTGTGA | AAAGTCGAAT | AACCATCAAC | |
| CCAGACACAT | CCAAGAACCA | GTTCTCCCTG | CAGCTGAACT | CTGTGACTCC | |
| CGAGGACACG | GCTGTGTATT | ACTGTACAAG | AGTGGGATAT | AGCAGTGGCT | |
| GGGGGGGCTA | CTACTACGGT | ATGGACGTCT | GGGGCCAAGG | GACCACGGTC | |
| ACCGTCTCCT | CAGGGAGTGC | ATCCGCCCCA | ACCCTTTTCC | CCCTCGTCTC | |
| CTGTGAGAAT | TCCCCGTCGG | ATACGAGCAG | CGTGGCCGTT | GGCTG | |

3E7 heavy chain VDJ region

| | | | | | |
|---|---|---|---|---|---|
| TGGGTTCACC | GTCAGTAGCA | ACTACATNAG | CTGGGTCCGC | CAGGCTCCAG | (SEQ ID NO: 32) |
| GGAAGGGGCT | GGAATGGGTN | TCAGTTATTT | ATACCGGTGG | TAACACATAC | |
| TACGCAGACT | CCGTNAAGGG | CCGATTCACC | ATCTCCAGAG | ACAATTCCAA | |
| GAACACGTTG | TATCTTCAAA | TGAACAGCCT | GAGAGCCNAG | GACACGGCCG | |
| TGTATTACTG | TGCGACTGGA | TTTAGTGGCT | ACGACTACTA | CTACTACGGT | |
| TTGGACGTCT | GGGGCCAAGG | GACCACGGTC | ACCGTCTCCT | CAGCCTCCAC | |
| CAAGGGCCCA | TCGGTCTTCC | CCCTGGCGCC | CTGCTCCAGG | AGCACCTCCN | |
| AGAGCACANC | GGCCCTGGGC | TGCCTGGTCA | AGGACTACTT | CCCCGAACCG | |
| GTGACGGTGT | CGTGGAACTC | AGGCGCTCTG | ACCAGCGGCA | TGCACACCGA | |
| G | | | | | |

In Table 1, N = G, A, T, C, other or unknown.

The amino acid sequences of the VDJ regions of the heavy chains OF mAbs 2E6 and 2C8 and the VJ regions of the light chains of mabs 2C8, 4G6 and 2G8 are provided in Table 2.

TABLE 2

Amino acid sequence of the VDJ region of the heavy
chain of mAb 2E6 (SEQ ID NO: 33)
QLXESGGGLVQPGGSLRLSCAASGFTFSXYAMXWVRQAPGTGLXWVSTINGTGXS
TYYADSVKGRFTISRDNSKNTLYLQMNSLRAEDTAXYYCAKDQYSSHYYYYGMDV
WGQGTTVTVSSASTKGPSVFPLAPCSRSTSESTAALGCLVKD
Amino acid sequence of the VDJ region of the heavy
chain of mAb 2C8 (SEQ ID NO: 34)
EVQLVESGGGLVQPGGSLRLSCAASGFTXSSYWMSWVRQAPGKGLEWVANIRQDG
SEKYYVDSVKGRFTISRDNAKNSLYLQMNSLRAEDTAVYYCARDRVAVAAPYYYY
YGLDVWGQGTTVTVSSASTKGPSVFPLAPCSRSTSESTAALGCLVKD
Amino acid sequence of the VJ region of the light chain
of mAb 2C8 (SEQ ID NO: 35)
GDRVTITCRASQSISTYLNWYQQKPGKAPKLLIYAASSLQSGVPSRFSGSGSGTD
FTLTISSLQTEDFATYFCQQRYNTPFTFGPGTKVDIKRTVAAPSVFIFPPSDEQL
KSGTASVVCLLNNFYPRE
Amino acid sequence of the VJ region of the light chain
of mAb 4G6 (SEQ ID NO: 36)
PSSLSASVGDRVTITCRASQGIRNNLGWYQQKPGKAPKRLIYAASSLQSGVPSRF
SGSGSGTEFTLTISSLQPEDFATYYCLQHNSYPCSFGQGTKLEIKRTVAAPSVFI
FPPSD
Amino acid sequence of the VJ region of the light chain
of mAb 2G8 (SEQ ID NO: 37)
SLPVTLGQPASISCRSSQSLVYSDGNTYLNWFQQRPGQSPRRLIYKVSNWDSGVP
DRFSGSGSGTDFTLKISRVEAEDVGVYYCMQGTHWP

In Table 2, X = any amino acid.

The CDR3-junction amino acid sequences of the kappa light chains of mabs 2E6, 4D11, 1C7, 2C8, 3B4, 4G6, 4C2, 2E11 and 3B10 are provided in Table 3. Each of the CDR regions of the kappa light chains of 4D11, 2C8, 4G6 and 2E11 has one mutation when compared to the germline kappa light chains.

TABLE 3

CDR3-junction amino acid sequence
of the kappa light
chain of mAb 2E6 (SEQ ID NO: 38)
CQKYNSAPFTFGPG
CDR3-junction amino acid sequence
of the kappa light
chain of mAb 4D11 (SEQ ID NO: 39)
CQQANSFPFTFGPG
CDR3-junction amino acid sequence
of the kappa light
chain of mAb 1C7 (SEQ ID NO: 40)
CQQYYSTPRTFGQG
CDR3-junction amino acid sequence
of the kappa light
chain of mAb 2C8 (SEQ ID NO: 41)
CQQRYNTPFTFGPG
CDR3-junction amino acid sequence
of the kappa light
chain of mAb 3B4 (SEQ ID NO: 42)
CLQHNSYPRTFGQG
CDR3-junction amino acid sequence
of the kappa light
chain of mAb 4G6 (SEQ ID NO: 43)
CLQHNSYPCSFGQG
CDR3-junction amino acid sequence
of the kappa light
chain of mAb 4C2 (SEQ ID NO: 44)
CQQYYSTPWTFGQG
CDR3-junction amino acid sequence
of the kappa light
chain of mAb 2E11 (SEQ ID NO: 45)
CMQGTHWPITFGQG
CDR3-junction amino acid sequence
of the kappa light
chain of mAb 3B10 (SEQ ID NO: 46)
CMQGTHWPCSFGQG

A CDR junction sequence may have all of that CDR sequence or most of that CDR sequence. A CDR junction sequence may have residues just 5′ and/or just 3′ to that CDR sequence.

Table 4 provides the amino acid sequences of the VDJ regions of the heavy chains and the VJ regions of the light chains of most of the 13 human anti-mitochondrial antigen mAbs derived from XENOMOUSE® animals (described in Examples 1-3 and FIGS. 6-8).

TABLE 4

The amino acid sequence of the VJ region of the light
chain of mAb 4C2 (SEQ ID NO: 47):
QSPSSVSASVGDRVTITC [RASRGISSWLA] WYQQKPGKAPKLLIF (AASSLQS)
GVPSRFSGSGSGTDFTLTISSLQPEDFATYFC {QQANSFP}
FTFGPGTKVDIKRTVAAPSVFIFPPSDEQLKSGTASVVCLLNNF
The sequence within [ ] is CDR1. The sequence within
( ) is CDR2. The sequence within { } is CDR3.
The amino acid sequence of the VJ region of the light
chain of mAb 2E6 (SEQ ID NO: 48):
GDRVXITC [RASQGISNYLA] WYQQKPGKVPKLLIY (XASTLQS) GVPSRFSGSG
SGTDFTLTISSLQPEDVATYYC {QKYNSAP} FTFGPGTKVDIKRTVAAPSVFI
FPPSDEQLIKSGTASVVCLLNNFY
The sequence within [ ] is CDR1. The sequence within
( ) is CDR2. The sequence within { } is CDR3.
The amino acid sequence of the VJ region of the light
chain of mAb 3E7 (SEQ ID NO: 49):
PSSLSASVGDRVTITC [RASQGISNYLA] WYQQKPGKVPKLLIY (TASTLQS) GV
PSRFSGSGSGTDFTFTISSLQPEDVATYYC {QKYNTAP} FTFGPGTKVDIKRTVA
APSVFIFPPSDEQLKSGTASVVCLLNNFYPRE
The sequence within [ ] is CDR1. The sequence within
( ) is CDR2. The sequence within { } is CDR3.
The amino acid sequence of the VJ region of the light
chain of mAb 2C8 (SEQ ID NO: 50):
GDRVTITC [RASQSISTYLN] WYQQKPGKAPKLLIY (AASSLQS) GVPSRFSGSG
SGTDFTLTISSLQTEDFATYFC {QQRYNTP} FTFGPGTKVDIKRTVAAPSVFIFP
PSDEQLKSGTASVVCLLNNFYPRE
The sequence within [ ] is CDR1. The sequence within
( ) is CDR2. The sequence within { } is CDR3.
The amino acid sequence of the VJ region of the light
chain of mAb 1C7 (SEQ ID NO: 51):
ATINC [KSSQSVLYSSNNKNYLA] WYQQKPGQPPKLLIY (WASTRES) GVPDRFS
GSGSGTDFTLTISSLQAEDVAVYYC {QQYYSTP} RTFGQGTKVEIKRTVAAPSVF
IFPPSDEQLKSGTASVVCLLNN
The sequence within [ ] is CDR1. The sequence within
( ) is CDR2. The sequence within { } is CDR3.
The amino acid sequence of the VJ region of the light
chain of mAb 4D11 (SEQ ID NO: 52):
PLSLPVTPGXPASISC [RSSQSLLHSNGYTYLD] WYLQKPGQSPQLLIY (LGSNR
AS) GVPDRFSGSGSGTDFTLKISRVEAEDVGVYYC {MQALQIP} WTFGQGTKVEI
KRTVAAPSVFIFPPSDEQLKSGTASXVCLLNNFL
The sequence within [ ] is CDR1. The sequence within
( ) is CDR2. The sequence within { } is CDR3.
The amino acid sequence of the VJ region of the light
chain of mAb 2G8 (SEQ ID NO: 53):
SLPVTLGQPASISC [RSSQSLVYSDGNTYLN] WFQQRPGQSPRRLIY
(KVSNWDS) GVPDRFSGSGSGTDFTLKISRVEAEDVGVYYC {MQGTHWP}
The sequence within [ ] is CDR1. The sequence within
( ) is CDR2. The sequence within { } is CDR3.
The amino acid sequence of the VJ region of the light
chain of mAb 2E11 (SEQ ID NO: 54):
LGQPASISC [RSSQSLVYSDGNTFLN] WFQQRPGQSPRRLIY (KVSNWDS) GVPD
RFSGSGSGTDFTLKISRVEAEDVGVYYC {MQGTHWP} ITFGQGTRLEIKRTVAAP
SVFIFPPSDEQLKSGTASVVCLLNNFY
The sequence within [ ] is CDR1. The sequence within
( ) is CDR2. The sequence within { } is CDR3.
The amino acid sequence of the VDJ region of the heavy
chain of mAb 3B4 (SEQ ID NO: 55):
IRQSPGKGLEWIG (HIYYSGNTNYNPSLKS) RLTISIDTSKTQFSLKLSS
VTAADTAIYYCVR {DRVTGAFDI} WGQGTMVTVSSASTKGPSVFPLAPCSRSTSE
STAALGCLVKDYFPEPVT
The amino acid sequence of the VDJ region of the heavy
chain of mAb 4C2 (SEQ ID NO: 56):
SGGSVS [SGDYYWT] WIRQSPGKGLEWIG (HIYYSGNTNRLYNPSLKS) RLTISI
DTSKTQFSLKLSSVTAADTAIYYCVR {DRVTGAFDI} WGQGTMVTVSSA
The amino acid sequence of the VDJ region of the heavy
chain of mAb 4G6 (SEQ ID NO: 57):
GLVKPSQTLSLTCAISGDSVS [SNSAAWN] WIRQSPSRGLEWLG (RTYYRSKWYN
DSAVSVKS) RITINPDTSKNQFSLQLNSVTPEDTAVYYCAS {WIAAAGFDS}
WGQGTLVTVSSGSASAPTLFPLVSCENSPSDTSSVAVG
The amino acid sequence of the VDJ region of the heavy
chain of mAb 2G8 (SEQ ID NO: 58):
PGLVKPSQTLSLTCAISGDSVS [SNSAAWN] WIRQSPSRGLEWLG (RTYYRSKWY
NDYTVSVKS) RITINPDTSKNQFSLQLNSVTPEDTAVYYCTR
{VGYSSGWGGYYYGMDV} WGQGTTVTVSSGSASAPTLFPLVSCENSPSDTSSVAV
G
The amino acid sequence of the VDJ region of the heavy
chain of mAb 2E11 (SEQ ID NO: 59):
EVQLLESGGGLVQPXGSLRXSCAAXGFT [FSSYAMT] WVRQAPGKGLEWVS (AIS
GSGGSTYYADSVKG) RFTISRDNSENTLYLQMNSLRAEDTAVYYCAN {VSFQL} W
GQGTLVTVSSGSASAPTLFPLVSCENSPSDTSSVAVGCLAQDFLPDSITFYL
The amino acid sequence of the VDJ region of the heavy
chain of mAb 1C7 (SEQ ID NO: 60):
DVQLLEXGGGLVQPXGXLRLSCAAXGFT [FSSYAMX] WVRQAXGKGLEWVS
(AISGSGGSTYYADSVKG) RFTISRDNSKNTLYLQMNSLRAEDTAVYYCAK {RW
QWLVDNGYYYYYGMDV} WGQGTTVTVSSGSASAPTLFPLVSCENSPSD
The amino acid sequence of the VDJ region of the heavy
chain of mAb 2E6 (SEQ ID NO: 61):
QLXESGGGLVQPGGSLRLSCAASGFT [FSXYAMX] WVRQAPGTGLXWVS (TINGT
GXSTYYADSVKG) RFTISRDNSKNTLYLQMNSLRAEDTAXYYCAK {DQYSSHYYY
YGMDV} WGQGTTVTVSSASTKGPSVFPLAPCSRSTSESTAALGCLVKD
The amino acid sequence of the VDJ region of the heavy
chain of mAb 4D11 (SEQ ID NO: 62):
QVQLVESGGGVVQPXRXLRXXCAASGFT [FSSYGMH] WVRQAXGKGLEWVA (VIW
YDGSNKYYADSVKG) RFTISRDNSKNTLYLQMNSLRAEDTAVYYCAR {SPADWYF
DL} WGRGTLVTVSSASTKGPSVFPLAPCSRSTSESTAALGCLVKD
The amino acid sequence of the VDJ region of the heavy
chain of mAb 2C8 (SEQ ID NO: 63):
EVQLVESGGGLVQPGGSLRLSCAASGFT [XSSYWMS] WVRQAPGKGLEWVA (NIR
QDGSEKYYVDSVKG) RFTISRDNAKNSLYLQMNSLRAEDTAVYYCAR {DRVAVAA
PYYYYYGLDV} WGQGTTVTVSSASTKGPSVFPLAPCSRSTSESTAALGCLVKD
The amino acid sequence of the VDJ region of the heavy
chain of mAb 3E7 (SEQ ID NO: 64):
EVQVVESGGGLIQPGGSLRLSCAASGFT [VSRNYMN] WVRQAPGKGLEWVS (VIY
SGGNTYYADSVKG) RFTISRDNSKNTLYLQMNSLRAEDTAVYYCAT {GXSGYDYY
YYGMDV} WGQGTTVTVSSASTKGPSVFPLAPCSRSTSESTAALGCLVKDXX
PEPVT

The CDR1, CDR2 and CDR3 sequences in Table 4 may be CDR1, CDR2 and CDR3 junction sequences. A CDR junction sequence may have all of that CDR sequence or most of that CDR sequence. A CDR junction sequence may have residues just 5′ and/or just 3′ to that CDR sequence.
In Table 4, X = any amino acid.

### Example 2

### Immunohistochemistry using the Human Mabs Obtained from XENOMOUSE® Animals and Described in Example 1

HEp2 Cells

We tested the thirteen human anti-mitochondrial antigen antibodies, obtained from XENOMOUSE® animals as described in Example 1, by immunohistochemistry using HEp-2 cell slides (Antibodies Inc., Davis, Calif.). Sections were incubated at RT for 1 hr with a human mAb (e.g., such as one of the thirteen human mabs described in Example 1) diluted at 1:5, XENOMOUSE® sera, sera from PBC patients or normal human control sera diluted at at least 1:40. After incubation, the slides were washed in PBS for 10 min. The reaction was followed by a 30-min incubation with FITC-conjugated goat anti-human IgG in PBS. All sections were viewed by a fluorescent microscope or confocal laser microscopy.

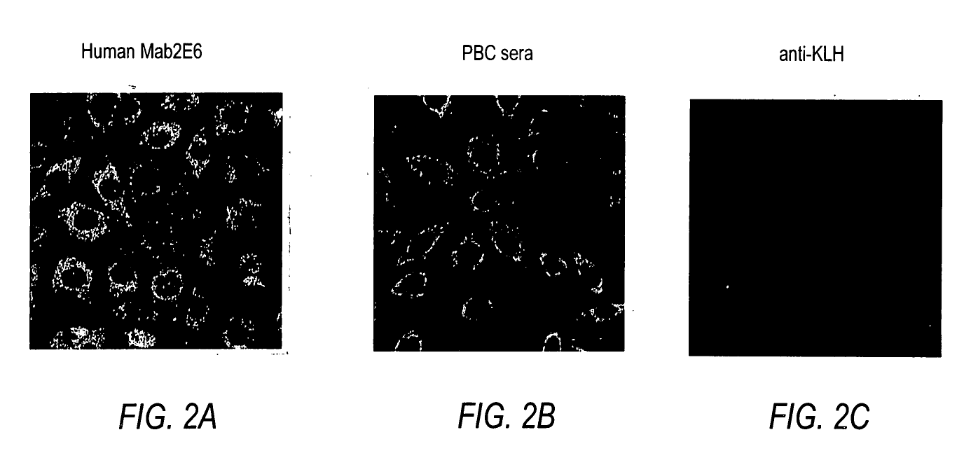

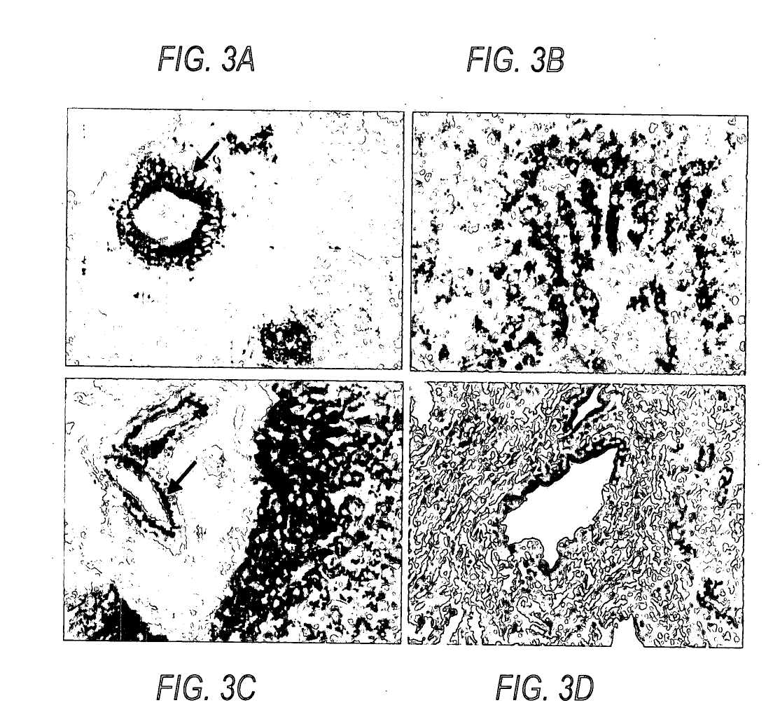

All thirteen human anti-mitochondrial antigen mabs obtained from XENOMOUSE® animals showed a cytoplasmic coarse-speckled pattern corresponding to mitochondrial staining on HEp-2 cell with variable intensities (FIGS. 2A, 2B and 2C) (FIG. 7). It is interesting to note that the human mAbs obtained from XENOMOUSE® mice that did not react with MIT3 showed less reactivity on HEp-2 cells (FIG. 6) than the human mabs obtained from XENOMOUSE® mice that recognized lipoyl domains in MIT3. This may be due to an affinity difference between the antibodies and may reflect the properties of the immunizing antigen, particularly the presence of the lipoyl moiety which is associated with an adjuvant effect. Alternatively, this may reflect the accessibility of the epitope in the native antigen, as MIT3 only contains limited epitopic regions of the enzymes. Control human mAb against KLH did not show any staining on HEp-2 cells (FIG. 2C). These data demonstrate the antigen specificity of this panel of human monoclonal antibodies to mitochondrial antigens.

Liver Tissue Sections

Freshly-frozen liver tissue sections were obtained from 10 patients with PBC. Explant tissues from 3 patients with primary sclerosing cholangitis (“PSC”) were used for disease specificity control. Tissues from two healthy donors were also used as controls. In addition, formalin-fixed and paraffin-embedded human liver samples were obtained from 13 patients with PBC, 3 patients with PSC, and 2 healthy donors removed for liver transplantation. The liver tissues from the thirteen patients with PBC were histologically classified as one with stage 2, seven with stage 3, and five patients with stage 4. All liver tissues from PSC patients were cirrhotic (stage 4). In addition, 5 samples of salivary gland, stomach, colon, lung, and heart were collected from surgical and autopsy files of Department of Pathology (II), Kanazawa University.

Human mAbs, such as the thirteen anti-mitochondrial antigen mabs, obtained from XENOMOUSE® animals, described in Example 1, were labeled with digoxigenin (DIG) using the DIG-antibody labeling kit (Roche). The DIG-labeled antibodies were purified using a centrifuge concentrator (Centricon) with several washing with Tris-buffered saline (pH7.6, TBS). The specificity and efficiency of DIG-labeled antibodies were checked by ELISA and HRP-labeled (HRP=horseradish peroxidase) anti-DIG antibody (Roche). For immunohistochemistry, 4 μm sections were mounted onto saline-coated slides to prevent tissue detachment. Deparaffinized and rehydrated sections or frozen tissue sections were fixed with cold acetone for 10 min and subsequently immersed in 1% blocking solution diluted in TBS for 30 min before incubating with each DIG-labeled Human mabs for 1 hr. After washing, the sections were incubated with alkaline-phosphatase (AP) labeled anti-DIG secondary antibody for 30 min. The alkaline phosphatase reaction was detected with NBT/BCIP solution (Roche) including 5 mM levamisol (Vector) for blocking an endogenous AP activity. Dig-labeled anti-KLH human MAb was used as control.

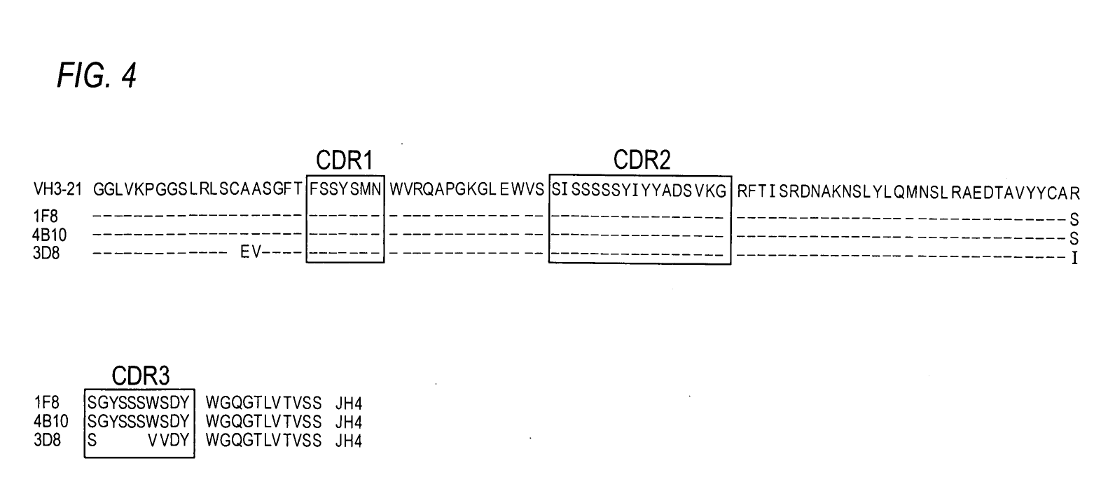

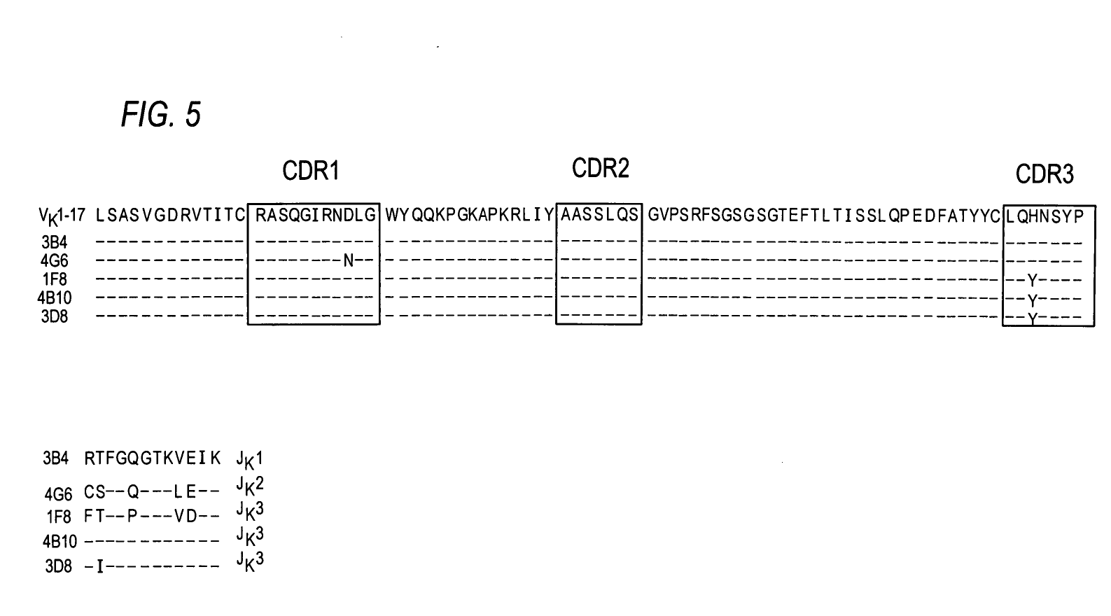

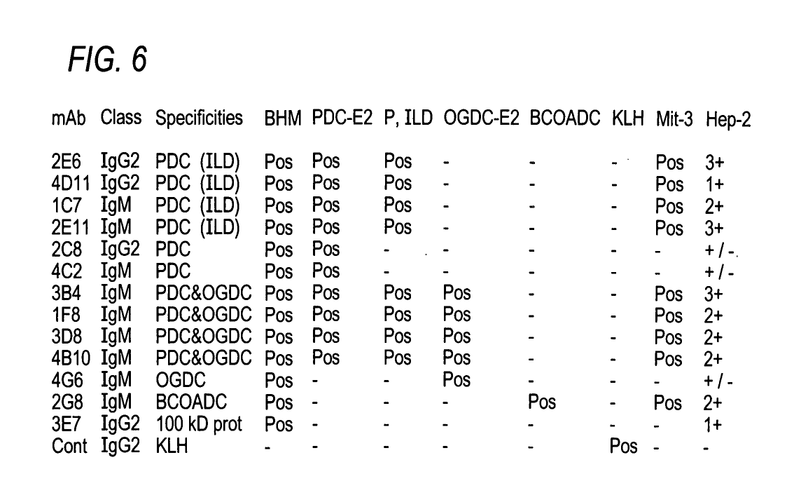

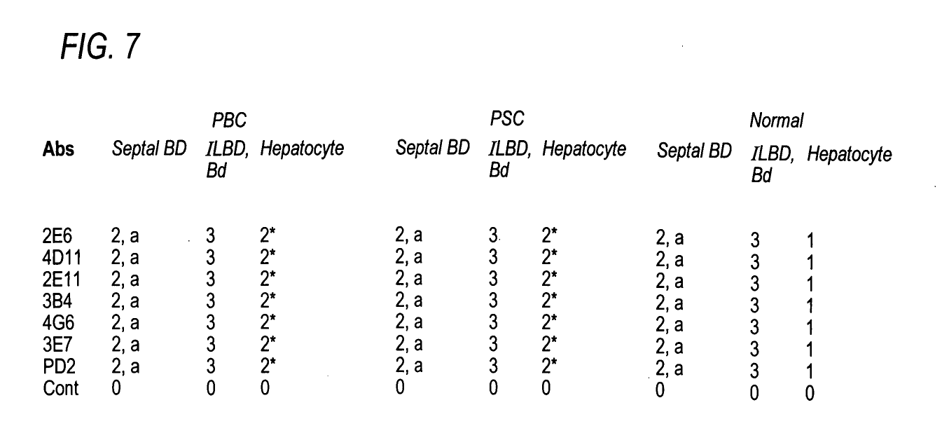

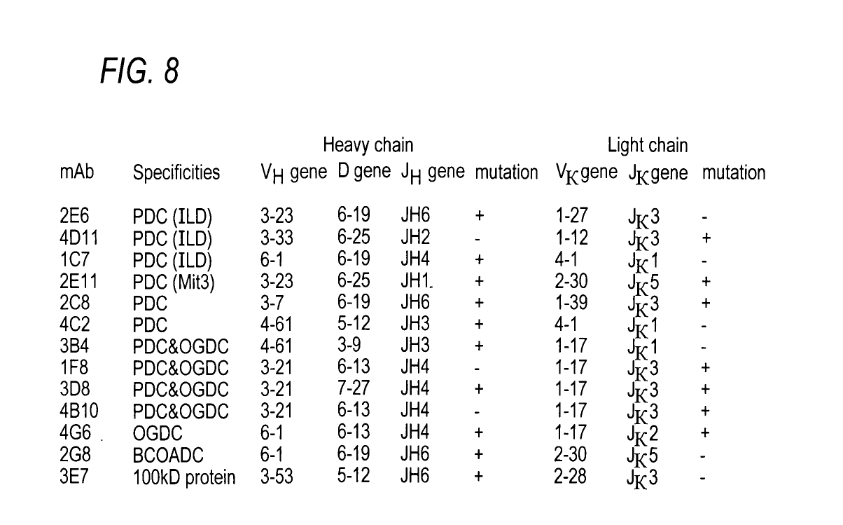

There was no significant difference in the staining pattern of the thirteen anti-mitochondrial antigens human mabs, obtained from XENOMOUSE® animals, described in Example 1 (staining pattern examples shown in FIG. 7). We observed Cytoplasmic staining with an accentuation at the apex of the cell in biliary epithelial cells of the septal bile ducts (examples shown in FIGS. 3A, 3B, 3C and 3D). In smaller interlobular bile ducts (ILBDs) and bile ductules, positive staining was seen diffusely in the cytoplasm and the staining intensity was a little higher than that seen in septal bile ducts (examples shown in FIGS. 3A, 3B, 3C and 3D). There was no difference between PBC livers and control livers in the staining pattern of the septal bile ducts, ILBDs and bile ductules. Hepatocytes showed diffuse cytoplasmic staining in normal livers and the staining intensity was relatively low compared to the staining in the bile ducts. In liver tissue sections from patients with PBC and PSC, especially those sections from patients in the cirrhotic stage of disease, intensely stained hepatocytes, which were a little smaller in size, were scattered in the hepatic parenchyma (examples shown in FIGS. 3A, 3B, 3C and 3D). The control human mAb against KLH did not stain PBC, PSC or normal liver (FIG. 7).

Other Tissues

The anti-mitochondrial antigen human mAbs obtained from XENOMOUSE® animals (the thirteen described in Example 1) were used to stain tissues from other organs, including salivary gland, stomach, colon, lung, and heart. Similar staining patterns of these tissues were observed with all thirteen human mabs obtained from XENOMOUSE® animals (the thirteen described in Example 1), although the staining varied in intensity. Among the organs studied, the ducts of salivary gland were most intensely stained, the intensity being similar to the staining of bile ducts by these human mAbs. In addition, smooth muscle cells in the vascular walls and epithelial cells and glands in stomach, colon, lung and heart were positively stained, although with a lower intensity than the staining of the bile ducts. Human mAb 4G6 stained mucus and fibrous tissue.

Despite the similarities noted herein between these human mabs obtained from XENOMOUSE® animals and autoantibodies from patients with PBC, the human autoantibody response in PBC and the human mabs produced here differ in their staining pattern in histochemistry.

Several murine mabs and human combinatorial mabs against PDC-E2 have been reported to stain the apical region of bile duct epithelial cells in PBC liver, but not PSC or normal livers. See Van de Water J et al., J. Clin. Invest. 91:2653-2664 (1993); Cha S, et al., Hepatology 20:574-583 (1994) and Cha S, et al., Proc Natl Acad Sci USA 93:10949-10954 (1995). In the present case, however, bile ducts in both PBC and control livers are similarly stained by these human mAbs. It should be noted that apical staining mAbs were rare among murine anti-mitochondrial antigens mAbs (Surh C D et al., J. Immunol. 144:2647-2652 (1990)) and such antibodies, are the exception rather than a rule, perhaps representing a subpopulation of affinity-matured antibodies with unique specificity.

It is possible that very early in the process of PBC development the human autoantibodies would more closely resemble those obtained from XENOMOUSE® animals. However, our previous reagents produced apical staining only in the intralobular small bile ducts and ductules and not in the septal ducts, which is clearly different from the staining pattern of the human mAbs obtained from XENOMOUSE® mice. The XENOMOUSE® mice raise antibodies that recognize common epitopes, whereas antisera and human and rare mouse mAbs may react with a modified region. What the modification might be and whether it is involved in the etiology of PBC or is a response to immune damage is unknown. Of interest, small scattered hepatocytes in cirrhotic nodules of PBC and PSC livers but not normal livers, and early stage of PBC and PSC livers were also strongly stained. This may be a reflection of increased mitochondrial activity in these proliferating cells in response to cirrhosis. It should be noted that 3E7, which recognizes an unknown 100 kD protein, also showed a similar staining pattern to the other OADC specific Human mAbs (FIG. 7). It appears that the staining pattern of this panel of human mabs from the XENOMOUSE® mice reflects the distribution and relative density of mitochondria in the target tissues.

Intense staining by the antibodies obtained from XENOMOUSE® animals is observed within ducts and ductules of salivary glands, gastric fundic glands, vessel walls and smooth muscle cell layer. The presence of staining in the salivary glands of PBC patients by anti-mitochondrial antigen antibodies have been previously reported (Tsuneyama K et al., Autoimmunity 26:23-31 (1997)). In particular, the staining was observed in both PBC patients with and without Sjögren's syndrome with similar frequency. This data and the recent evidence of presence of IgG and IgA anti-mitochondrial antigen antibodies in the saliva of PBC patients (Reynosa-Paz S et al., Hepatoloqy 31:24-29 (2000)) together support the earlier observation and hypothesis that a PDC-E2 molecule might be aberrantly expressed in the salivary glands of PBC patients and the possible association of Sjögren's syndrome with PBC (Tsuneyama K et al., Autoimmunity 26:23-31 (1997)).

### Example 3

### IG Gene Usage Determination of the Human Mabs Obtained from XENOMOUSE® Animals and Described in Example 1

We prepared Poly A(+) mRNA from the human mAb clones (i.e., of the thirteen anti-mitochondrial antigen human mAbs, obtained from XENOMOUSE® animals, described in example 1) using the Micro-Fast Track and Fast-Track 2.0 kits for the isolation of poly (A+) RNA. The PCR amplification protocol and V_{H} family-specific primers and V_{L} primers have been previously described. See Green L, J Exp Med 188:483-495 (1998) and Marks J et al., Eur J Immunol 21:985-991 (1991). Nucleotide sequencing was performed using 4.75% acrylamide gels, a Prism dye terminator sequencing kit and the 373 DNA sequencer (Applied Biosystems). Sequences were analyzed with MacVector and Gene Works software. The V base human antibody data base was used for sequence alignments and gene segment identifications (Tomlinson et al., MRC Centre for Protein Engineering, Cambridge, UK). DNA sequences were aligned sequentially to identify first the V_{H} and then the J_{H} segment. The intervening sequence that had no homology with either the V_{H} or J_{H} segments was then aligned against a database of human D segments in V base and the best alignment was identified.

FIG. 8 lists the Ig germline gene closest in sequence to each of the genes encoding the anti-mitochondrial antigen human mAbs obtained from XENOMOUSE® animals (described in Example 1). For Ig H chain, V_{H}3 (8/13), V_{H}4 (2/13), and V_{H}6 (3/13) families are used in the mitochondrial antigen specific human mAbs. Specifically, mabs 2E6 and 2E11 use V_{H}3-23; mAbs 1F8, 3D8, and 4B10 use V_{H}3-21; mAb 4D11 uses V_{H}3-33; mAb 2C8 uses V_{H}3-7; mAb 3E7 uses V_{H}3-53; mAbs 4C2 and 3B4 use V_{H}4-61; and mAbs 1C7, 4G6 and 2G8 use V_{H}6-1.

As shown in FIG. 8, four of the thirteen clones (2E6, 1C7, 2C8, 2G8) use a D6-19 gene; three (1F8, 4B10, 4G6) use a D6-13 gene; two (4D11, 2E11) use a D6-25 gene; one (3B4) uses a D3-9 gene and one (3D8) uses a D7-27 gene.

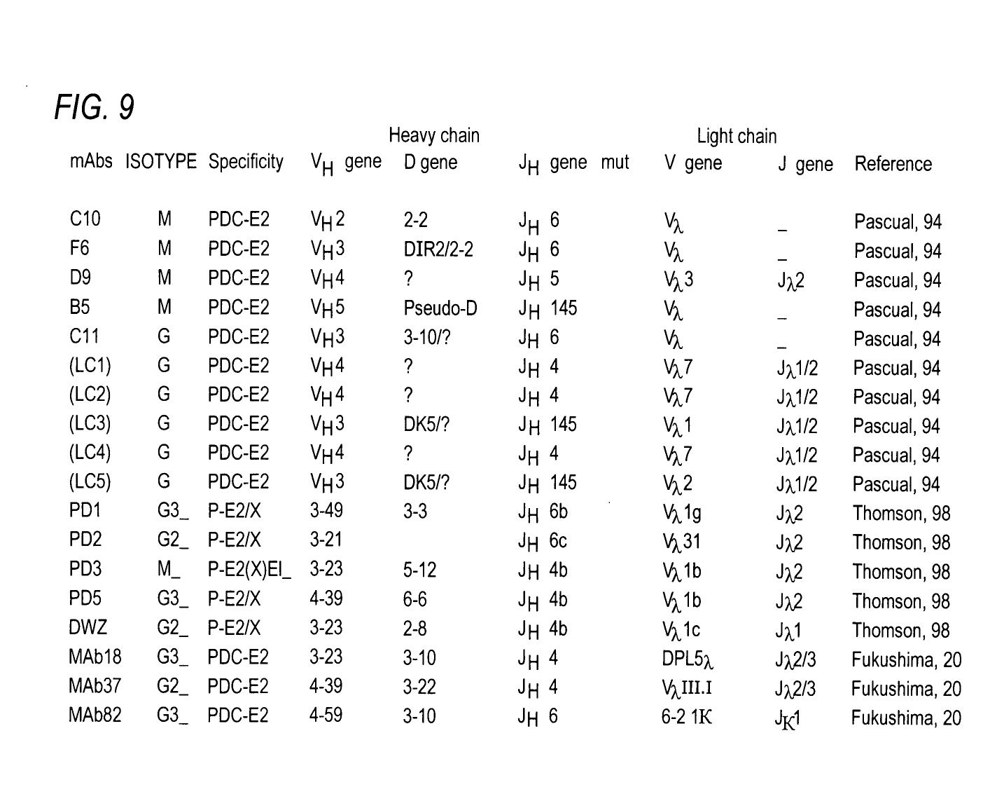

As shown in FIG. 8, five of the thirteen clones use J_{H}4 and four of the thirteen clones use J_{H}6 genes. Two clones use J_{H}3, and the remaining two use J_{H}1 and J_{H}2, respectively. Nine of the thirteen clones have replacement (R) mutation in their CDR regions. Human mAbs 1F8 and 4B10 have the same gene sequence using V_{H}3-21, D6-13, and J_{H}4 (FIG. 4). 3D8 uses V_{H}3-21 and J_{H}4, which is the same usage as 1F8 and 4B10, although the D gene usage is different (FIG. 4).

Regarding the sequence of the κ light chain, all human mAbs obtained from XENOMOUSE® animals (described in Example 1) recognizing both PDC-E2 and OGDC as well as 4G6, which reacts with OGDC only, use Vκ1-17 (FIG. 5). Human mAbs 1F8, 4B10 and 3D8 use the same Jκ3 gene, and the sequence of 1F8 and 4B10 are identical at this region, whereas there are some differences found in 3D8 (FIG. 5). FIG. 5 shows a comparison of the Jκ3 region of human mAbs 1F8, 4B10 and 3D8. The sequence of human mAbs 3B4 and 4G6 are almost the same except for a few differences in the CDR3 region. Both human mAbs 1C7 and 4C2 use Vκ4-1 and Jκ1 and have only a single amino acid difference between them in their CDR3. Vκ2-30 and Jκ5 are used in clones 2E11 and 2G8. Six of the thirteen clones have replacement (R) mutations in the CDR regions of the K chain.

In studies with other antigens, the V, D, and J segment utilization in XENOMOUSE® mice are nearly identical to the gene segment utilization reported for humans (Gallo M et al., Eur J Immunol 30:534-540 (2000)). Several interesting trends can be seen with respect to the Ig gene repertoire usage of these human mabs. First, V_{H}3, V_{H}4 and V_{H}6 Ig gene segments are used in the human mAbs clones generated from XENOMOUSE® mice (FIG. 8) and there is no preferential Ig gene usage with respect to antigen specificity. Second, the 1F8 and 4B10 Ig sequence are nearly identical and 3D8 has a similar sequence except for a slight difference in the D_{H} region. These 3 clones were generated in the same fusion, and appear to originate from an identical clone in the immunized XENOMOUSE® mice. Third, the 4 clones that recognize both PDC-E2 and OGDC-E2, and the additional OGDC-E2-specific mAb 4G6, all use V_{K}1-17, which is likely due to antigen-driven selection of the V_{K} in these antibodies. Fourth, V_{H}3-21 (3/13), 3-23 (2/13), 4-61(2/13), 6-1(3/13), DH6-19, 6-13 and 5-12 J_{H}4 and J_{H}6 are used more frequently by the anti-mitochondrial antigen human mabs. In contrast, V_{H}3-23, 3-30, 3-33, V_{H}4-4, 4-31, 4-34 and 4-59 as well as J_{H}4 and J_{H}6 are frequently used by non-immunized XENOMOUSE® mice and human (Mendez M et al., Nat Genet 15:146-156 (1997) and Gallo M et al., Eur J Immunol 30:534-540 (2000)). The gene usage of the anti-mitochondrial antigens human mAbs suggest that these antibodies are the result of antigen driven selection.

Some of the V_{H} genes used in the anti-mitochondrial antigen human mAbs are also used in autoantibodies with other specificities in autoimmune diseases such as lupus and rheumatoid arthritis. See He X et al., Mol Med 1:768-780 (1995); Huang S C et al., Clin Exp Immunol 112:516-527 (1998) and Mahmoudi M et al., Lupus 6:578-589 (1997). Although it has been reported that V_{H}3 and V_{H}4 with highly mutated CDR are utilized in anti-mitochondrial antigen antibodies (Fukushima N et al., Int. Immunol.7:1047-1055 (1995); Pascual V et al., J. Immunol. 152:2577-2585 (1994) and Thomson R K et al., J. Hepatology 28:582-594 (1998)) and V_{H}3-21, V_{H}3-23 are used also in both the patient and the XENOMOUSE® mice derived anti-mitochondrial antigens human mabs, the CDRs of the antibodies are distinct. Analysis of the Ig gene repertoire used by the human monoclonal antibodies generated herein (described in Examples 1-3) showed a restricted repertoire in their CDRs. In addition, most of these antibodies are IgM and are not hypermutated when compared to their germline genes, suggesting that these antibodies might represent naturally occurring autoantibodies, which are of lower affinity. In addition, since human anti-PDC-E2 monoclonal antibodies of both κ and λ light chains have been reported (Cha S et al., Proc Natl Acad Sci USA 90:2527-2531 (1993); Cha S et al., Hepatology 20:574-583 (1994) and Fukushima N et al., J Autoimmune 14:247-257 (2000)); the generation of human mabs from XENOMOUSE® mice, which are limited to κ light chains might impose certain unknown restrictions on the affinity and maturation of these antibodies.

All publications, patents and patent applications cited in this specification are herein incorporated by reference as if each individual publication, patent or patent application were specifically and individually indicated to be incorporated by reference.

Biological Deposits

The following hybridomas (which are mouse hybridomas) expressing the antibodies as indicated below

cell line 4B10 (mAb 4B10): ATCC Accession No. ,
cell line 1F8 (mAb 1E8): ATCC Accession No. ,
cell line 4G6 (mAb 4G6): ATCC Accession No. ,
cell line 3D8 (mAb 3DB): ATCC Accession No. ,
cell line 3B4 (mAb 3B4): ATCC Accession No. ,
cell line 4C2 (mAb 4C2): ATCC Accession No. ,
cell line 2E6 (mAb 2E6): ATCC Accession No. ,
cell line 3E7 (mAb 3E7): ATCC Accession No. ,
cell line 2C8 (mAb 2C8): ATCC Accession No. ,
cell line 1C7 (mAb 1C7): ATCC Accession No. ,
cell line 4D11 (mAb 4D11): ATCC Accession No. ,
cell line 2G8 (mAb 2G8): ATCC Accession No. ,
and
cell line 2E11 (mAb 2E11): ATCC Accession No. ,
were deposited with the American Type Culture
Collection (“ATCC”), 10801 University Boulevard,
Manassas, VA 20110-2209, USA, on ,
and given the above-indicated ATCC Accession Numbers.

Equivalents

The invention may be embodied in other specific forms without departing from the spirit or essential characteristics thereof. The foregoing embodiments are therefore to be considered in all respects illustrative of, rather than limiting on, the invention disclosed herein.

What is claimed is:

1. A method of making an isolated human monoclonal antibody, or antigen-binding portions thereof, that binds a mitochondrial antigen bound by a human autoantibody found in patients with primary biliary cirrhosis comprising the steps of:
a) providing a transgenic non-human animal that has the ability to make human antibodies;
b) immunizing said animal with said mitochondrial antigen;
c) obtaining a hybridoma cell line making a human antibody to said antigen; and
d) isolating said human antibody to said antigen.

2. The method according to claim 1, wherein said animal is deficient in the ability to make said animal's antibodies.

3. The method according to claim 1, wherein said transgenic non-human animal is a mouse.

4. The method according to claim 3, wherein said mouse is a XENOMOUSE® mouse.

5. The method according to claim 1, wherein said mitochondrial antigen is selected from the group consisting of the E2 subunit of the pyruvate dehydrogenase complex, the E2 subunit of the branched-chain 2-oxo-acid dehydrogenase complex, the E2 subunit of the 2-oxoglutarate dehydrogenase complex and E3-binding protein.

6. The method according to claim 1, wherein said isolated human antibody binds to one or more peptides, polypeptides or proteins selected from the group consisting of the inner lipoyl domain of the E2 subunit of pyruvate dehydrogenase complex; the E2 subunit of the pyruvate dehydrogenase complex; the E2 subunit of the 2-oxoglutarate dehydrogenase complex; the E2 subunit of the branched-chain 2-oxo-acid dehydrogenase complex, MIT3 and p100 bound by monoclonal antibody 3E7.

7. An isolated human antibody, or antigen-binding portions thereof, that binds a mitochondrial antigen bound by a human autoantibody found in patients with primary biliary cirrhosis.

8. The isolated human antibody, or antigen-binding portions thereof, according to claim 7, wherein said human antibody is a monoclonal antibody.

9. The isolated human antibody, or antigen-binding portions thereof, according to claim 7, wherein said human antibody is produced by any one of the methods of claims 1-6.

10. The isolated human antibody, according to claim 7, wherein said human antibody is an IgG2 or an IgM antibody.

11. An isolated antibody, or antigen-binding portions thereof, that binds a mitochondrial antigen bound by a human autoantibody found in patients with primary biliary cirrhosis, wherein said antibody has one or more CDR1, CDR2 or CDR3 of any one of the amino acid sequence selected from the group consisting of: SEQ ID NO: 1, SEQ ID NO: 2, SEQ ID NO: 3, SEQ ID NO: 6, SEQ ID NO: 7, SEQ ID NO: 8, SEQ ID NO: 9 and SEQ ID NO: 10.

12. An isolated antibody, or antigen-binding portions thereof, that binds a mitochondrial antigen bound by a human autoantibody found in patients with primary biliary cirrhosis, wherein said antibody has a CDR3 of any one of the amino acid sequence selected from the group consisting of: SEQ ID NO: 1, SEQ ID NO: 2, SEQ ID NO: 3, SEQ ID NO: 6, SEQ ID NO: 7, SEQ ID NO: 8, SEQ ID NO: 9 and SEQ ID NO: 10.

13. An isolated antibody, or antigen-binding portions thereof, that binds a mitochondrial antigen bound by a human autoantibody found in patients with primary biliary cirrhosis, wherein said antibody, wherein said antibody has one or more non-germline FR1, FR2, FR3 or FR4 of any one of the amino acid sequence selected from the group consisting of: SEQ ID NO: 1, SEQ ID NO: 2, SEQ ID NO: 3, SEQ ID NO: 6, SEQ ID NO: 7, SEQ ID NO: 8, SEQ ID NO: 9 and SEQ ID NO: 10.

14. The isolated human antibody according to claim 7, wherein said human antibody comprises a heavy chain that utilizes a V_{H} gene selected from the group consisting of V_{H}3, V_{H}4 and V_{H}6.

15. The isolated human antibody, or antigen-binding portions thereof, according to claim 14, wherein said heavy chain has at least one mutation compared to the germline heavy chain.

16. The isolated human antibody, or antigen-binding portions thereof, according to claim 14, wherein said human antibody comprises a heavy chain that utilizes a V_{H}3 gene selected from the group consisting of V_{H}3-23, V_{H}3-21, V_{H}3-33, V_{H}3-7 and V_{H}3-53.

17. The isolated human antibody, or antigen-binding portions thereof, according to claim 14, wherein said human antibody comprises a heavy chain that utilizes a V_{H}4-61 gene.

18. The isolated human antibody, or antigen-binding portions thereof, according to claim 14, wherein said human antibody comprises a heavy chain that utilizes a V_{H}6-1 gene.

19. The isolated human antibody, or antigen-binding portions thereof, according to claim 7, wherein said human antibody comprises a light chain that utilizes a V_{K} gene selected from the group consisting of V_{K}1-12, V_{K}1-17, V_{K}1-27, V_{K}1-39, V_{K}2-28, V_{K}2-30, V_{K}4-1.

20. The isolated human antibody according to claim 19, wherein said light chain has at least one mutation compared to the germline light chain.

21. An isolated antibody, or antigen-binding portions thereof, having a non-germline heavy chain region of any one of the amino acid sequence selected from the group consisting of: SEQ ID NO: 1, SEQ ID NO: 2, SEQ ID NO: 3, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63 and SEQ ID NO: 64.

22. An isolated antibody, or antigen-binding portions thereof, having a non-germline light chain region of any one of the amino acid sequence selected from the group consisting of: SEQ ID NO: 6, SEQ ID NO: 7, SEQ ID NO: 8, SEQ ID NO: 9, SEQ ID NO: 10, SEQ ID NO: 47, SEQ ID NO: 48, SEQ ID NO: 49, SEQ ID NO: 50, SEQ ID NO: 51, SEQ ID NO: 52, SEQ ID NO: 53, and SEQ ID NO: 54.

23. An isolated antibody or antigen-binding portion thereof, wherein said antibody comprises a heavy chain VDJ region amino acid sequence and a light chain VJ region amino acid sequence selected from the group consisting of:
a) a heavy chain VDJ region amino acid sequence encoded by the nucleic acid sequence of SEQ ID NO: 28, and a light chain VJ region amino acid sequence encoded by the nucleic acid sequence of SEQ ID NO: 12, or said amino acid sequences encoded by said nucleic acid sequences encoding the amino acid sequences lacking the signal sequences;
(b) a heavy chain VDJ region amino acid sequence encoded by the nucleic acid sequence of SEQ ID NO: 29, and a light chain VJ region amino acid sequence encoded by the nucleic acid sequence of SEQ ID NO: 13, or said amino acid sequences encoded by said nucleic acid sequences encoding the amino acid sequences lacking the signal sequences;
(c) a heavy chain VDJ region amino acid sequence encoded by the nucleic acid sequence of SEQ ID NO: 24, and a light chain VJ region amino acid sequence encoded by the nucleic acid sequence of SEQ ID NO: 14, or said amino acid sequences encoded by said nucleic acid sequences encoding the amino acid sequences lacking the signal sequences;
(d) a heavy chain VDJ region amino acid sequence encoded by the nucleic acid sequence of SEQ ID NO: 26, and a light chain VJ region amino acid sequence encoded by the nucleic acid sequence of SEQ ID NO: 16, or said amino acid sequences encoded by said nucleic acid sequences encoding the amino acid sequences lacking the signal sequences;
(e) a heavy chain VDJ region amino acid sequence encoded by the nucleic acid sequence of SEQ ID NO: 32, and a light chain VJ region amino acid sequence encoded by the nucleic acid sequence of SEQ ID NO: 17, or said amino acid sequences encoded by said nucleic acid sequences encoding the amino acid sequences lacking the signal sequences;
(f) a heavy chain VDJ region amino acid sequence encoded by the nucleic acid sequence of SEQ ID NO: 25, and a light chain VJ region amino acid sequence encoded by the nucleic acid sequence of SEQ ID NO: 18, or said amino acid sequences encoded by said nucleic acid sequences encoding the amino acid sequences lacking the signal sequences;
(g) a heavy chain VDJ region amino acid sequence encoded by the nucleic acid sequence of SEQ ID NO: 22, and a light chain VJ region amino acid sequence encoded by the nucleic acid sequence of SEQ ID NO: 19, or said amino acid sequences encoded by said nucleic acid sequences encoding the amino acid sequences lacking the signal sequences;
(h) a heavy chain VDJ region amino acid sequence encoded by the nucleic acid sequence of SEQ ID NO: 23, and a light chain VJ region amino acid sequence encoded by the nucleic acid sequence of SEQ ID NO: 20, or said amino acid sequences encoded by said nucleic acid sequences encoding the amino acid sequences lacking the signal sequences; and
(i) a heavy chain VDJ region amino acid sequence encoded by the nucleic acid sequence of SEQ ID NO: 27, and a light chain VJ region amino acid sequence encoded by the nucleic acid sequence of SEQ ID NO: 21, or said amino acid sequences encoded by said nucleic acid sequences encoding the amino acid sequences lacking the signal sequences.

24. An isolated antibody or antigen-binding portion thereof, wherein said antibody comprises a heavy chain VDJ region amino acid sequence selected from of the group consisting of:
(a) a heavy chain VDJ region amino acid sequence encoded by the nucleic acid sequence of SEQ ID NO: 30, or said amino acid sequence encoded by said nucleic acid sequence encoding the amino acid sequence lacking the signal sequence; and
(b) a heavy chain VDJ region amino acid sequence encoded by the nucleic acid sequence of SEQ ID NO: 31, or said amino acid sequence encoded by said nucleic acid sequence encoding the amino acid sequence lacking the signal sequence.

25. An isolated antibody or antigen-binding portion thereof, wherein said antibody comprises a light chain VJ region amino acid sequence encoded by the nucleic acid sequence of SEQ ID NO: 15, or said amino acid sequence encoded by said nucleic acid sequence encoding the amino acid sequence lacking the signal sequence.

26. An isolated antibody or antigen-binding portion thereof, wherein said antibody comprises a light chain VJ region amino acid sequence encoded by the nucleic acid sequence of SEQ ID NO: 15, wherein said antibody comprises a heavy chain that utilizes a V_{H}3-23 gene, a D6-19 gene and a J_{H}6 gene and wherein said heavy chain has at least one mutation compared to the germline heavy chain, and wherein said antibody specifically binds to the E2 component of the pyruvate dehydrogenase complex (PDC-E2) and the inner lipoyl domain of PDC-E2.

27. An isolated antibody or antigen-binding portion thereof, wherein said antibody comprises a heavy chain VDJ region amino acid sequence encoded by the nucleic acid sequence of SEQ ID NO: 30, wherein said antibody comprises a light chain that utilizes a V_{K}1-17 gene and a J_{K}2 gene and wherein said light chain has at least one mutation compared to the germline light chain and wherein said antibody does not specifically binds to the E2 component of the pyruvate dehydrogenase complex (PDC-E2) but binds specifically to the E2 component of the 2-oxoglutarate dehydrogenase complex (OGDC-E2).

28. An isolated antibody or antigen-binding portion thereof, wherein said antibody comprises a heavy chain VDJ region amino acid sequence encoded by the nucleic acid sequence of SEQ ID NO: 31, wherein said antibody comprises a light chain that utilizes a V_{K}2-30 gene and a J_{K}5 gene and wherein said antibody does not specifically binds to the E2 component of the pyruvate dehydrogenase complex (PDC-E2) but binds specifically to the E2 component of the branched-chain 2-oxo-acid dehydrogenase complex (BCOADC-E2).

29. An isolated antibody or antigen-binding portion thereof, wherein said antibody comprises a light chain that utilizes a V_{K}1-39 gene and a J_{K}3 gene and wherein said light chain has at least one mutation compared to the germline light chain, wherein said antibody comprises a heavy chain that utilizes a V_{H}3-7 gene, a D6-19 gene and a J_{H}6 gene and wherein said heavy chain has at least one mutation compared to the germline heavy chain and wherein said antibody specifically binds to the E2 component of the pyruvate dehydrogenase complex (PDC-E2) but does not specifically bind to the inner lipoyl domain of PDC-E2.

30. An isolated antibody or antigen-binding portion thereof, wherein said antibody comprises a light chain VJ region amino acid sequence encoded by the nucleic acid sequence of SEQ ID NO: 15 and wherein said antibody comprises a heavy chain VDJ region amino acid sequence of SEQ ID NO: 33.

31. An isolated antibody or antigen-binding portion thereof, wherein said antibody comprises a heavy chain VDJ region amino acid sequence encoded by the nucleic acid sequence of SEQ ID NO: 30 and wherein said antibody comprises a light chain VJ region amino acid sequence of SEQ ID NO: 36.

32. An isolated antibody or antigen-binding portion thereof, wherein said antibody comprises a heavy chain VDJ region amino acid sequence encoded by the nucleic acid sequence of SEQ ID NO: 31 and wherein said antibody comprises a light chain VJ region amino acid sequence of SEQ ID NO: 37.

33. An isolated antibody or antigen-binding portion thereof, wherein said antibody comprises a heavy chain VDJ region amino acid sequence of SEQ ID NO: 34 and wherein said antibody comprises a light chain VJ region amino acid sequence of SEQ ID NO: 35.

34. An isolated antibody, or antigen-binding portion thereof, comprising a light chain comprising a CDR3-junction amino acid sequence selected from the group consisting of: a CDR3-junction amino acid sequence of SEQ ID NO: 38, a CDR3-junction amino acid sequence of SEQ ID NO: 39, a CDR3-junction amino acid sequence of SEQ ID NO: 40, a CDR3-junction amino acid sequence of SEQ ID NO: 41, a CDR3-junction amino acid sequence of SEQ ID NO: 42, a CDR3-junction amino acid sequence of SEQ ID NO: 43, a CDR3-junction amino acid sequence of SEQ ID NO: 44, a CDR3-junction amino acid sequence of SEQ ID NO: 45 and a CDR3-junction amino acid sequence of SEQ ID NO: 46.

35. An isolated antibody or antigen-binding portion thereof, wherein said antibody comprises a heavy chain VDJ amino acid sequence and a light chain VJ amino acid sequence selected from the group consisting of:
(a) a heavy chain VDJ region amino acid sequence of SEQ ID NO: 1, and a light chain VJ region amino acid sequence encoded by the nucleic acid sequence of SEQ ID NO: 8, or said amino acid sequences lacking the signal sequences;
(b) a heavy chain VDJ region amino acid sequence of SEQ ID NO: 2, and a light chain VJ region amino acid sequence encoded by the nucleic acid sequence of SEQ ID NO: 9, or said amino acid sequences lacking the signal sequences;
(c) a heavy chain VDJ region amino acid sequence of SEQ ID NO: 3, and a light chain VJ region amino acid sequence encoded by the nucleic acid sequence of SEQ ID NO: 10, or said amino acid sequences lacking the signal sequences;
(d) a heavy chain VDJ region amino acid sequence of SEQ ID NO: 55, and a light chain VJ region amino acid sequence encoded by the nucleic acid sequence of SEQ ID NO: 6, or said amino acid sequences lacking the signal sequences;
(e) a heavy chain VDJ region amino acid sequence of SEQ ID NO: 56, and a light chain VJ region amino acid sequence encoded by the nucleic acid sequence of SEQ ID NO: 47, or said amino acid sequences lacking the signal sequences;
(f) a heavy chain VDJ region amino acid sequence of SEQ ID NO: 57, and a light chain VJ region amino acid sequence encoded by the nucleic acid sequence of SEQ ID NO: 7, or said amino acid sequences lacking the signal sequences;
(g) a heavy chain VDJ region amino acid sequence of SEQ ID NO: 58, and a light chain VJ region amino acid sequence encoded by the nucleic acid sequence of SEQ ID NO: 53, or said amino acid sequences lacking the signal sequences;
(h) a heavy chain VDJ region amino acid sequence of SEQ ID NO: 59, and a light chain VJ region amino acid sequence encoded by the nucleic acid sequence of SEQ ID NO: 54, or said amino acid sequences lacking the signal sequences;
(i) a heavy chain VDJ region amino acid sequence of SEQ ID NO: 60, and a light chain VJ region amino acid sequence encoded by the nucleic acid sequence of SEQ ID NO: 51, or said amino acid sequences lacking the signal sequences;
(j) a heavy chain VDJ region amino acid sequence of SEQ ID NO: 61, and a light chain VJ region amino acid sequence encoded by the nucleic acid sequence of SEQ ID NO: 48, or said amino acid sequences lacking the signal sequences;
(k) a heavy chain VDJ region amino acid sequence of SEQ ID NO: 62, and a light chain VJ region amino acid sequence encoded by the nucleic acid sequence of SEQ ID NO: 52, or said amino acid sequences lacking the signal sequences;
(l) a heavy chain VDJ region amino acid sequence of SEQ ID NO: 63 and a light chain VJ region amino acid sequence encoded by the nucleic acid sequence of SEQ ID NO: 50, or said amino acid sequences lacking the signal sequences; and
(m) a heavy chain VDJ region amino acid sequence of SEQ ID NO: 64, and a light chain VJ region amino acid sequence encoded by the nucleic acid sequence of SEQ ID NO: 49, or said amino acid sequences lacking the signal sequences.

36. The isolated antibody according to any one of claims 11-13, 21-35, wherein said antibody is a human antibody.

37. The isolated antibody according to any one of claims 11-13, 21-35, wherein said antibody is a monoclonal antibody.

38. The isolated human antibody according claim 36, wherein said antibody is a monoclonal antibody.

39. A DNA molecule encoding an immunoglobulin comprising an amino acid sequence selected from the group consisting of: SEQ ID NO: 1, SEQ ID NO: 2, SEQ ID NO: 3, SEQ ID NO: 6, SEQ ID NO: 7, SEQ ID NO: 8, SEQ ID NO: 9 and SEQ ID NO: 10.

40. A DNA molecule encoding an immunoblogulin comprising a heavy chain VDJ region amino acid sequence and light chain VJ region amino acid sequence selected from the group consisting of:
(a) a heavy chain VDJ region amino acid sequence encoded by the nucleic acid sequence of SEQ ID NO: 28, and a light chain VJ region amino acid sequence encoded by the nucleic acid sequence of SEQ ID NO: 12, or said amino acid sequences encoded by said nucleic acid sequences encoding the amino acid sequences lacking the signal sequences;
(b) a heavy chain VDJ region amino acid sequence encoded by the nucleic acid sequence of SEQ ID NO: 29, and a light chain VJ region amino acid sequence encoded by the nucleic acid sequence of SEQ ID NO: 13, or said amino acid sequences encoded by said nucleic acid sequences encoding the amino acid sequences lacking the signal sequences;
(c) a heavy chain VDJ region amino acid sequence encoded by the nucleic acid sequence of SEQ ID NO: 24, and a light chain VJ region amino acid sequence encoded by the nucleic acid sequence of SEQ ID NO: 14, or said amino acid sequences encoded by said nucleic acid sequences encoding the amino acid sequences lacking the signal sequences;
(d) a heavy chain VDJ region amino acid sequence encoded by the nucleic acid sequence of SEQ ID NO: 26, and a light chain VJ region amino acid sequence encoded by the nucleic acid sequence of SEQ ID NO: 16, or said amino acid sequences encoded by said nucleic acid sequences encoding the amino acid sequences lacking the signal sequences;
(e) a heavy chain VDJ region amino acid sequence encoded by the nucleic acid sequence of SEQ ID NO: 32, and a light chain VJ region amino acid sequence encoded by the nucleic acid sequence of SEQ ID NO: 17, or said amino acid sequences encoded by said nucleic acid sequences encoding the amino acid sequences lacking the signal sequences;
(f) a heavy chain VDJ region amino acid sequence encoded by the nucleic acid sequence of SEQ ID NO: 25, and a light chain VJ region amino acid sequence encoded by the nucleic acid sequence of SEQ ID NO: 18, or said amino acid sequences encoded by said nucleic acid sequences encoding the amino acid sequences lacking the signal sequences;
(g) a heavy chain VDJ region amino acid sequence encoded by the nucleic acid sequence of SEQ ID NO: 22, and a light chain VJ region amino acid sequence encoded by the nucleic acid sequence of SEQ ID NO: 19, or said amino acid sequences encoded by said nucleic acid sequences encoding the amino acid sequences lacking the signal sequences;
(h) a heavy chain VDJ region amino acid sequence encoded by the nucleic acid sequence of SEQ ID NO: 23, and a light chain VJ region amino acid sequence encoded by the nucleic acid sequence of SEQ ID NO: 20, or said amino acid sequences encoded by said nucleic acid sequences encoding the amino acid sequences lacking the signal sequences; and
(i) a heavy chain VDJ region amino acid sequence encoded by the nucleic acid sequence of SEQ ID NO: 27, and a light chain VJ region amino acid sequence encoded by the nucleic acid sequence of SEQ ID NO: 21, or said amino acid sequences encoded by said nucleic acid sequences encoding the amino acid sequences lacking the signal sequences.

41. A DNA molecule encoding an immunoglobulin comprising an amino acid sequence selected from the group consisting of: SEQ ID NO: 47, SEQ ID NO: 48, SEQ ID NO: 49, SEQ ID NO: 50, SEQ ID NO: 51, SEQ ID NO: 52, SEQ ID NO: 53 and SEQ ID NO: 54.

42. A DNA molecule encoding an immunoglobulin comprising an amino acid sequence selected from the group consisting of: SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63 and SEQ ID NO: 64.

43. A DNA molecule encoding an immunoblogulin comprising a heavy chain VDJ region amino acid sequence and light chain VJ region amino acid sequence selected from the group consisting of:
a) a heavy chain VDJ region amino acid sequence of SEQ ID NO: 1, and a light chain VJ region amino acid sequence encoded by the nucleic acid sequence of SEQ ID NO: 8, or said amino acid sequences lacking the signal sequences;
b) a heavy chain VDJ region amino acid sequence of SEQ ID NO: 2, and a light chain VJ region amino acid sequence encoded by the nucleic acid sequence of SEQ ID NO: 9, or said amino acid sequences lacking the signal sequences;
c) a heavy chain VDJ region amino acid sequence of SEQ ID NO: 3, and a light chain VJ region amino acid sequence encoded by the nucleic acid sequence of SEQ ID NO: 10, or said amino acid sequences lacking the signal sequences;
d) a heavy chain VDJ region amino acid sequence of SEQ ID NO: 55, and a light chain VJ region amino acid sequence encoded by the nucleic acid sequence of SEQ ID NO: 6, or said amino acid sequences lacking the signal sequences;
e) a heavy chain VDJ region amino acid sequence of SEQ ID NO: 56, and a light chain VJ region amino acid sequence encoded by the nucleic acid sequence of SEQ ID NO: 47, or said amino acid sequences lacking the signal sequences;
f) a heavy chain VDJ region amino acid sequence of SEQ ID NO: 57, and a light chain VJ region amino acid sequence encoded by the nucleic acid sequence of SEQ ID NO: 7, or said amino acid sequences lacking the signal sequences;
g) a heavy chain VDJ region amino acid sequence of SEQ ID NO: 58, and a light chain VJ region amino acid sequence encoded by the nucleic acid sequence of SEQ ID NO: 53, or said amino acid sequences lacking the signal sequences;
h) a heavy chain VDJ region amino acid sequence of SEQ ID NO: 59, and a light chain VJ region amino acid sequence encoded by the nucleic acid sequence of SEQ ID NO: 54, or said amino acid sequences lacking the signal sequences;
i) a heavy chain VDJ region amino acid sequence of SEQ ID NO: 60, and a light chain VJ region amino acid sequence encoded by the nucleic acid sequence of SEQ ID NO: 51, or said amino acid sequences lacking the signal sequences;
j) a heavy chain VDJ region amino acid sequence of SEQ ID NO: 61, and a light chain VJ region amino acid sequence encoded by the nucleic acid sequence of SEQ ID NO: 48, or said amino acid sequences lacking the signal sequences;
k) a heavy chain VDJ region amino acid sequence of SEQ ID NO: 62, and a light chain VJ region amino acid sequence encoded by the nucleic acid sequence of SEQ ID NO: 52, or said amino acid sequences lacking the signal sequences;
l) a heavy chain VDJ region amino acid sequence of SEQ ID NO: 63 and a light chain VJ region amino acid sequence encoded by the nucleic acid sequence of SEQ ID NO: 50, or said amino acid sequences lacking the signal sequences; and
m) a heavy chain VDJ region amino acid sequence of SEQ ID NO: 64, and a light chain VJ region amino acid sequence encoded by the nucleic acid sequence of SEQ ID NO: 49, or said amino acid sequences lacking the signal sequences.

44. A isolated cell line that secretes a human antibody that specific binds a mitochondrial antigen bound by a human autoantibody found in patients with primary biliary cirrhosis, wherein said cell line is a hybridoma cell line produced by any one of the methods of claims 1-6, steps a)-c) or wherein said cell line secretes any one of the antibodies of claims 7-38 or the immunoglobulins of claim 39-43.

45. The cell line according to claim 44, wherein said cell line is a hybridoma cell line.

46. A transgenic non-human animal that makes a human antibody that binds a mitochondrial antigen bound by a human autoantibody found in patients with primary biliary cirrhosis, wherein said transgenic non-human animal has the ability to make human antibodies.

47. The transgenic non-human animal according to claim 46, wherein said animal is deficient in the ability to make said animal's antibodies.

48. The transgenic non-human animal according to claim 46, wherein said transgenic animal is a mouse.

49. The transgenic non-human animal according to claim 48, wherein said mouse is a XENOMOUSE® mouse.

50. The transgenic non-human animal according to claim 46, wherein said mitochondrial antigen is selected from the group consisting of the E2 subunit of the pyruvate dehydrogenase complex, the E2 subunit of the branched-chain 2-oxo-acid dehydrogenase complex, the E2 subunit of the 2-oxoglutarate dehydrogenase complex and E3-binding protein.

51. The transgenic non-human animal according to claim 46, wherein said isolated human antibody binds to one or more peptide, polypeptide or protein selected from the group consisting of the inner lipoyl domain of the E2 subunit of pyruvate dehydrogenase complex; the E2 subunit of the pyruvate dehydrogenase complex; the E2 subunit of the 2-oxoglutarate dehydrogenase complex; the E2 subunit of the branched-chain 2-oxo-acid dehydrogenase complex, MIT3 and p100.

52. A method of obtaining an antagonist of a human antibody that binds to a mitochondrial antigen bound by a human autoantibody found in patients with primary biliary cirrhosis, comprising the step of screening for said antagonist in a transgenic non-human animal according to any one of claims 46-51, wherein said antagonist reduces the production of said antibody in said transgenic non-human animal.

53. A method of obtaining a antagonist of an isolated human antibody that binds to a mitochondrial antigen bound by a human autoantibody found in patients with primary biliary cirrhosis, comprising the step of screening for said antagonist in an in vitro assay, wherein said antagonist interrupts the interaction of an antibody according to any one of claims 7-38 or the immunoglobulin according to claims 39-43 with one or more antigens that said antibody binds.

54. A method of treating or preventing primary biliary cirrhosis, comprising the step of modifying or blocking the epitope on some or all mitochondrial antigens for a human antibody that binds to mitochondrial antigens bound by a human autoantibody found in patients with primary biliary cirrhosis.

55. A method of treating or preventing primary biliary cirrhosis, comprising the step of administering the antagonist identified in any one of claims 52-53 comprising the step of administering a pharmaceutical composition comprising said antagonist to a subject in need thereof.

56. An isolated protein bound by human monoclonal antibody 3E7, said antibody having a heavy chain VDJ region encoded by a nucleic acid molecule comprising a nucleic acid sequence of SEQ ID NO: 32 and a light chain VJ region encoded by a nucleic acid molecule comprising a nucleic acid sequence of SEQ ID NO: 17, wherein said isolated protein is a mitochondrial protein and has a molecular weight of approximately 100 kilodaltons as determined by SDS-PAGE.

57. An isolated nucleic acid molecule comprising a nucleic acid sequence selected from the group consisting of: SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20 and SEQ ID NO: 21.

58. An isolated nucleic acid molecule comprising a nucleic acid sequence selected from the group consisting of: SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31 and SEQ ID NO: 32.

59. An isolated, recombinant or synthetic DNA or polynucleotide comprising a nucleic acid sequence that hybridizes under stringent conditions to and that is at least 80% complementary to any of the nucleic acid sequences selected from the group consisting of: SEQ ID NO: 22, SEQ ID NO: 23, SEQ ID NO: 24, SEQ ID NO: 25, SEQ ID NO: 26, SEQ ID. NO: 27, SEQ ID NO: 28, SEQ ID NO: 29, SEQ ID NO: 30, SEQ ID NO: 31 and SEQ ID NO: 32.

60. An isolated nucleic acid molecule comprising a nucleic acid sequence that hybridizes under stringent conditions to and that is at least 80% complementary to any of the nucleic acid sequences selected from the group consisting of: SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20 and SEQ ID NO: 21.

61. An isolated nucleic acid molecule comprising a nucleic acid sequence that is at least 60% homologous to any of the nucleic acid sequence selected from the group consisting of: SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31 and SEQ ID NO: 32.

62. An isolated nucleic acid molecule comprising a nucleic acid sequence that is at least 60% homologous to any of the nucleic acid sequence selected from the group consisting of: SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20 and SEQ ID NO: 21.


SEQUENCE LISTING (64 sequences)

SEQ ID NO: 1 - protein, 111 aa, Artificial Sequence
  Description of Artificial Sequence Human mAb
GGLVKPGGSLRLSCAASGFTFSSYSMNWVRQAPGKGLEWVSSISSSSSYIYYADSVKGRFTISRDNAKNSLYLQMNSLRAEDTAVYYCASSGYSSSWSDYWGQGTLVTVSS

SEQ ID NO: 2 - protein, 111 aa, Artificial Sequence; identical to SEQ ID NO: 1
  Description of Artificial Sequence Human mAb

SEQ ID NO: 3 - protein, 105 aa, Artificial Sequence
  Description of Artificial Sequence Human mAb
GGLVKPGGSLRLSEVSGFTFSSYSMNWVRQAPGKGLEWVSSISSSSSYIYYADSVKGRFTISRDNAKNSLYLQMNSLRAEDTAVYYCAISVVDYWGQGTLVTVSS

SEQ ID NO: 4 - protein, 90 aa, Homo sapiens
GGLVKPGGSLRLSCAASGFTFSSYSMNWVRQAPGKGLEWVSSISSSSSYIYYADSVKGRFTISRDNAKNSLYLQMNSLRAEDTAVYYCAR

SEQ ID NO: 5 - protein, 15 aa, Artificial Sequence
  Description of Artificial Sequence Synthetic Gly- Ser linker
GGGGSGGGGSGGGGS

SEQ ID NO: 6 - protein, 97 aa, Artificial Sequence
  Description of Artificial Sequence Human mAb
LSASVGDRVTITCRASQGIRNDLGWYQQKPGKAPKRLIYAASSLQSGVPSRFSGSGSGTEFTLTISSLQPEDFATYYCLQHNSYPRTFGQGTKVEIK

SEQ ID NO: 7 - protein, 97 aa, Artificial Sequence
  Description of Artificial Sequence Human mAb
LSASVGDRVTITCRASQGIRNNLGWYQQKPGKAPKRLIYAASSLQSGVPSRFSGSGSGTEFTLTISSLQPEDFATYYCLQHNSYPCSFGQGTKLEIK

SEQ ID NO: 8 - protein, 97 aa, Artificial Sequence
  Description of Artificial Sequence Human mAb
LSASVGDRVTITCRASQGIRNDLGWYQQKPGKAPKRLIYAASSLQSGVPSRFSGSGSGTEFTLTISSLQPEDFATYYCLQYNSYPFTFGPGTKVDIK

SEQ ID NO: 9 - protein, 97 aa, Artificial Sequence; identical to SEQ ID NO: 8
  Description of Artificial Sequence Human mAb

SEQ ID NO: 10 - protein, 97 aa, Artificial Sequence
  Description of Artificial Sequence Human mAb
LSASVGDRVTITCRASQGIRNDLGWYQQKPGKAPKRLIYAASSLQSGVPSRFSGSGSGTEFTLTISSLQPEDFATYYCLQYNSYPFIFGPGTKVDIK

SEQ ID NO: 11 - protein, 85 aa, Homo sapiens
LSASVGDRVTITCRASQGIRNDLGWYQQKPGKAPKRLIYAASSLQSGVPSRFSGSGSGTEFTLTISSLQPEDFATYYCLQHNSYP

SEQ ID NO: 12 - DNA, 441 bp, Artificial Sequence
  Description of Artificial Sequence Human mAb
tcctccctgtctgcatctgttggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattattgtctacagtataatagttacccattcattttcggccctgggaccaaagtggatatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaa

SEQ ID NO: 13 - DNA, 435 bp, Artificial Sequence
  Description of Artificial Sequence Human mAb
cctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagtataatagttacccattcactttcggccctgggaccaaagtggatatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctcca

SEQ ID NO: 14 - DNA, 415 bp, Artificial Sequence
  Description of Artificial Sequence Human mAb
gcccgtcacccttggacagccggcctccatctcctgcaggtctagtcaaagcctcgtatacagtgatggaaacaccttcttgaattggtttcagcagaggccaggccaatctccaaggcgcctaatttataaggtttctaactgggactctggggtcccagacagattcagcggcagtgggtcaggcactgatttcacactgaaaatcagcagggtggaggctgaggatgttggggtttattactgcatgcaaggtacacactggcctatcaccttcggccaagggacacgactggagattaaacgaactgtggctgcaccatctgtcttcatcttcccgccatctgatgagcagttgaaatctggaactgcctctgttgtgtgcctgctgaataacttctatcccagagagggc

SEQ ID NO: 15 - DNA, 391 bp, Artificial Sequence
  Description of Artificial Sequence Human mAb
tgtaggagacagagtcaccatcacttgccgggcgagtcagggcattagcaattatttagcctggtatcagcagaaaccagggaaagttcctaagctcctgatctatgctgcatccactttgcaatcaggggtcccatctcggttcagtggcagtggatctgggacagatttcactctcaccatcagcagcctgcagcctgaagatgttgcaacttattactgtcaaaagtataacagtgccccattcactttcggccctgggaccaaagtggatatcaaacgaactgtggctgcaccatctgtcttcatcttcccgccatctgatgagcagttgaaatctggaactgcctctgttgtgtgcctgctgaataacttctataacagagaggng

SEQ ID NO: 16 - DNA, 389 bp, Artificial Sequence
  Description of Artificial Sequence Human mAb
gtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatagttaccctcggacgttcggccaagggaccaaggtggaaatcaaacgaactgtggctgcaccatctgtcttcatcttcccgccatctgatgagcagttgaaatctggaactgcctctgttgtgtgcctgctgaataacttctatacccagagagg

SEQ ID NO: 17 - DNA, 389 bp, Artificial Sequence
  Description of Artificial Sequence Human mAb
atctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactccgtggacgttcggccaagggaccaaggtggaaatcaaacgaactgtggctgcaccatctgtcttcatcttcccgccatctgatgagcagttgaaatctggaactgcctctgttgtgtgcctgctgaataacttctatancanagaggaggg

SEQ ID NO: 18 - DNA, 413 bp, Artificial Sequence
  Description of Artificial Sequence Human mAb
tctgggcgagagggccaccatcaactgcaagtccagccagagtgttttatacagctccaacaataagaactacttagcttggtaccagcagaaaccaggacagcctcctaagttgctcatttactgggcatctacccgggaatccggggtccctgaccgattcagtggcagcgggtctgggacagatttcactctcaccatcagcagcctgcaggctgaagatgtggcagtttattactgtcagcaatattatagtactccgtggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggggatag

SEQ ID NO: 19 - DNA, 383 bp, Artificial Sequence
  Description of Artificial Sequence Human mAb
agagtcaccatcacttgtcgggcgagtcggggtattagcagctggttagcctggtatcagcagaaaccagggaaagcccctaagctcctgatctttgctgcatccagtttgcaaagtggggtcccatcaaggttcagcggcagtggatctgggacagatttcactctcaccatcagcagcctgcagcctgaagattttgcaacttacttttgtcaacaggctaacagtttcccattcactttcggccctgggaccaaagtggatatcaaacgaactgtggctgcaccatctgtcttcatcttcccgccatctgatgagcagttgaaatctggaactgcctctgttgtgtgcctgctgaataacttctatcccagagagggcat

SEQ ID NO: 20 - DNA, 426 bp, Artificial Sequence
  Description of Artificial Sequence Human mAb
gtgtctctgggcgagagggccaccatcaactgcaagtccagccagagtgttttatacagctccaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagtactcctcggacgttcggccaagggaccaaggtggaaatcaaacgaactgtggctgcaccatctgtcttcatcttcccgccatctgatgagcagttgaaatctggaactgcctctgttgtgtgcctgctgaataacttctataacagagagggatnccaaagagtc

SEQ ID NO: 21 - DNA, 435 bp, Artificial Sequence; identical to SEQ ID NO: 13
  Description of Artificial Sequence Human mAb

SEQ ID NO: 22 - DNA, 446 bp, Artificial Sequence
  Description of Artificial Sequence Human mAb
ggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaatactatgcagactccgtgaagggccgattcaccatctccagagacaattccaagaacacgctgtatctgcaaatgaacagcctgagagccgaggacacggctgtgtattactgtgcgagatctcctgcggactggtacttcgatctctggggccgtggcaccctggtcactgtctcctcagcctccaccaagggcccatcggtcttccccctggcgccctgctccaggagcacctccgagagcacagcggccctgggctgcctggtcaaggantacttccccgaaccggtgacg

SEQ ID NO: 23 - DNA, 425 bp, Artificial Sequence
  Description of Artificial Sequence Human mAb
ccaggactggtgaagccctcgcagaccctctcactcacctgtgccatctccggggacagtgtctctagcaacagtgctgcttggaactggatcaggcagtccccatcgagaggccttgagtggctgggaaggacatactacaggtccaagtggtataatgattatgcagtatctgtgaaaagtcgaataaccatcaacccagacacatccaagaaccagttctccctgcagctgaactctgtgactcccgaggacacggctgtgtattactgtgcaagagggtatagcagtggctggtactttgactactggggccagggaaccctggtcaccgtctcctcagggagtgcatccgccccaacccttttccccctcgtctcctgtgagaattccccgtcggatacgagcagcgtggccgttggctg

SEQ ID NO: 24 - DNA, 390 bp, Artificial Sequence
  Description of Artificial Sequence Human mAb
gatccggcagtccccagggaagggactggagtggattggacacatctattacagtgggaacaccaattataacccctccctcaagagtcgactcaccatatcaattgacangtccaagactcagttctccctgaagctgagttctgtgaccgctgcggacacggccatttattactgtgtgcnagatcgagtgactggtgcttttgatatctggggccaagggacaatggtcaccgtctcttcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcgg

SEQ ID NO: 25 - DNA, 378 bp, Artificial Sequence
  Description of Artificial Sequence Human mAb
tctctggtggctccgtcagcagtggtgattactactggacctggatccggcagtccccagggaagggactggagtggattggacacatctattacagtgggaacaccaattataacccctccctcaagagtcgactcaccatatcaattgacacgtccaagactcagttctccctgaagctgagttctgtgaccgctgcggacacggccatttattactgtgtgcgagatcgagtgactggtgcttttgatatctggggccaagggacaatggtcaccgtctcttcagcctccaccaagggcccatcggtcttccccctggcgccctgctccaggagcacctccgagagcacagcggccctgggctgcctggtcaagg

SEQ ID NO: 26 - DNA, 366 bp, Artificial Sequence
  Description of Artificial Sequence Human mAb
gatccggcagtccccagggaagggactggagtggattggacacatctattacagtgggaacaccaattataacccctccctcaagagtcgactcaccatatcaattgacacgtccaagactcagttctccctgaagctgagttctgtgaccgctgcggacacggccatttattactgtgtgcgagatcgagtgactggtgcttttgatatctggggccaagggacaatggtcaccgtctcttcagcctccaccaagggcccatcggtcttccccctggcgccctgctccaggagcacctccgagagcacagcggccctgggctgcctggtcaaggactacttccccgaaccggtgacggtgtcgtg

SEQ ID NO: 27 - DNA, 417 bp, Artificial Sequence
  Description of Artificial Sequence Human mAb
gggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagctccgggtatagcagcagctggtctgactactggggccagggaaccctggtcaccgtctcctcagggagtgcatccgccccaacccttttccccctcgtctcctgtgagaattccccgtcggatacgagcagcgtggccgttggctg

SEQ ID NO: 28 - DNA, 403 bp, Artificial Sequence
  Description of Artificial Sequence Human mAb
ggggaggcctggtcaagcctggggggtccctgagactctcctgtgaagtctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgatatcagtggttgactactggggccagggaaccctggtcaccgtctcctcagggagtgcatccgccccaacccttttccccctcgtctcctgtgagaattccccgtcggatacgagcagcgtggccgttggctg

SEQ ID NO: 29 - DNA, 417 bp, Artificial Sequence; identical to SEQ ID NO: 27
  Description of Artificial Sequence Human mAb

SEQ ID NO: 30 - DNA, 421 bp, Artificial Sequence
  Description of Artificial Sequence Human mAb
caggactggtgaagccctcgcagaccctctcactcacctgtgccatctccggggacagtgtctctagcaacagtgctgcttggaactggatcaggcagtccccatcgagaggccttgagtggctgggaaggacatactacaggtccaagtggtataatgattctgcagtatctgtgaaaagtcgaataaccatcaacccagacacatccaagaaccagttctccctgcagctgaactctgtgactcccgaggacacggctgtgtattactgtgcaagctggattgcagcagctggttttgactcctggggccagggaaccctggtcaccgtctcctcagggagtgcatccgccccaacccttttccccctcgtctcctgtgagaattccccgtcggatacgagcagcgtggccgttggctg

SEQ ID NO: 31 - DNA, 445 bp, Artificial Sequence
  Description of Artificial Sequence Human mAb
gtccaggactggtgaagccctcgcagaccctctcactcacctgtgccatctccggggacagtgtctctagcaacagtgctgcttggaactggatcaggcagtccccatcgagaggccttgagtggctgggaaggacatactacaggtccaagtggtataatgattatacagtatctgtgaaaagtcgaataaccatcaacccagacacatccaagaaccagttctccctgcagctgaactctgtgactcccgaggacacggctgtgtattactgtacaagagtgggatatagcagtggctgggggggctactactacggtatggacgtctggggccaagggaccacggtcaccgtctcctcagggagtgcatccgccccaacccttttccccctcgtctcctgtgagaattccccgtcggatacgagcagcgtggccgttggctg

SEQ ID NO: 32 - DNA, 451 bp, Artificial Sequence
  Description of Artificial Sequence Human mAb
tgggttcaccgtcagtagcaactacatnagctgggtccgccaggctccagggaaggggctggaatgggtntcagttatttataccggtggtaacacatactacgcagactccgtnaagggccgattcaccatctccagagacaattccaagaacacgttgtatcttcaaatgaacagcctgagagccnaggacacggccgtgtattactgtgcgactggatttagtggctacgactactactactacggtttggacgtctggggccaagggaccacggtcaccgtctcctcagcctccaccaagggcccatcggtcttccccctggcgccctgctccaggagcacctccnagagcacancggccctgggctgcctggtcaaggactacttccccgaaccggtgacggtgtcgtggaactcaggcgctctgaccagcggcatgcacaccgag

SEQ ID NO: 33 - protein, 152 aa, Artificial Sequence
  Description of Artificial Sequence Human mAb
QLXESGGGLVQPGGSLRLSCAASGFTFSXYAMXWVRQAPGTGLXWVSTINGTGXSTYYADSVKGRFTISRDNSKNTLYLQMNSLRAEDTAXYYCAKDQYSSHYYYYGMDVWGQGTTVTVSSASTKGPSVFPLAPCSRSTSESTAALGCLVKD

SEQ ID NO: 34 - protein, 157 aa, Artificial Sequence
  Description of Artificial Sequence Human mAb
EVQLVESGGGLVQPGGSLRLSCAASGFTXSSYWMSWVRQAPGKGLEWVANIRQDGSEKYYVDSVKGRFTISRDNAKNSLYLQMNSLRAEDTAVYYCARDRVAVAAPYYYYYGLDVWGQGTTVTVSSASTKGPSVFPLAPCSRSTSESTAALGCLVKD

SEQ ID NO: 35 - protein, 128 aa, Artificial Sequence
  Description of Artificial Sequence Human mAb
GDRVTITCRASQSISTYLNWYQQKPGKAPKLLIYAASSLQSGVPSRFSGSGSGTDFTLTISSLQTEDFATYFCQQRYNTPFTFGPGTKVDIKRTVAAPSVFIFPPSDEQLKSGTASVVCLLNNFYPRE

SEQ ID NO: 36 - protein, 115 aa, Artificial Sequence
  Description of Artificial Sequence Human mAb
PSSLSASVGDRVTITCRASQGIRNNLGWYQQKPGKAPKRLIYAASSLQSGVPSRFSGSGSGTEFTLTISSLQPEDFATYYCLQHNSYPCSFGQGTKLEIKRTVAAPSVFIFPPSD

SEQ ID NO: 37 - protein, 91 aa, Artificial Sequence
  Description of Artificial Sequence Human mAb
SLPVTLGQPASISCRSSQSLVYSDGNTYLNWFQQRPGQSPRRLIYKVSNWDSGVPDRFSGSGSGTDFTLKISRVEAEDVGVYYCMQGTHWP

SEQ ID NO: 38 - protein, 14 aa, Artificial Sequence
  Description of Artificial Sequence Human mAb
CQKYNSAPFTFGPG

SEQ ID NO: 39 - protein, 14 aa, Artificial Sequence
  Description of Artificial Sequence Human mAb
CQQANSFPFTFGPG

SEQ ID NO: 40 - protein, 14 aa, Artificial Sequence
  Description of Artificial Sequence Human mAb
CQQYYSTPRTFGQG

SEQ ID NO: 41 - protein, 14 aa, Artificial Sequence
  Description of Artificial Sequence Human mAb
CQQRYNTPFTFGPG

SEQ ID NO: 42 - protein, 14 aa, Artificial Sequence
  Description of Artificial Sequence Human mAb
CLQHNSYPRTFGQG

SEQ ID NO: 43 - protein, 14 aa, Artificial Sequence
  Description of Artificial Sequence Human mAb
CLQHNSYPCSFGQG

SEQ ID NO: 44 - protein, 14 aa, Artificial Sequence
  Description of Artificial Sequence Human mAb
CQQYYSTPWTFGQG

SEQ ID NO: 45 - protein, 14 aa, Artificial Sequence
  Description of Artificial Sequence Human mAb
CMQGTHWPITFGQG

SEQ ID NO: 46 - protein, 14 aa, Artificial Sequence
  Description of Artificial Sequence Human mAb
CMQGTHWPCSFGQG

SEQ ID NO: 47 - protein, 134 aa, Artificial Sequence
  Description of Artificial Sequence Human mAb
QSPSSVSASVGDRVTITCRASRGISSWLAWYQQKPGKAPKLLIFAASSLQSGVPSRFSGSGSGTDFTLTISSLQPEDFATYFCQQANSFPFTFGPGTKVDIKRTVAAPSVFIFPPSDEQLKSGTASVVCLLNNF

SEQ ID NO: 48 - protein, 125 aa, Artificial Sequence
  Description of Artificial Sequence Human mAb
GDRVXITCRASQGISNYLAWYQQKPGKVPKLLIYXASTLQSGVPSRFSGSGSGTDFTLTISSLQPEDVATYYCQKYNSAPFTFGPGTKVDIKRTVAAPSVFIFPPSDEQLKSGTASVVCLLNNFY

SEQ ID NO: 49 - protein, 136 aa, Artificial Sequence
  Description of Artificial Sequence Human mAb
PSSLSASVGDRVTITCRASQGISNYLAWYQQKPGKVPKLLIYTASTLQSGVPSRFSGSGSGTDFTFTISSLQPEDVATYYCQKYNTAPFTFGPGTKVDIKRTVAAPSVFIFPPSDEQLKSGTASVVCLLNNFYPRE

SEQ ID NO: 50 - protein, 128 aa, Artificial Sequence; identical to SEQ ID NO: 35
  Description of Artificial Sequence Human mAb

SEQ ID NO: 51 - protein, 126 aa, Artificial Sequence
  Description of Artificial Sequence Human mAb
ATINCKSSQSVLYSSNNKNYLAWYQQKPGQPPKLLIYWASTRESGVPDRFSGSGSGTDFTLTISSLQAEDVAVYYCQQYYSTPRTFGQGTKVEIKRTVAAPSVFIFPPSDEQLKSGTASVVCLLNN

SEQ ID NO: 52 - protein, 138 aa, Artificial Sequence
  Description of Artificial Sequence Human mAb
PLSLPVTPGXPASISCRSSQSLLHSNGYTYLDWYLQKPGQSPQLLIYLGSNRASGVPDRFSGSGSGTDFTLKISRVEAEDVGVYYCMQALQIPWTFGQGTKVEIKRTVAAPSVFIFPPSDEQLKSGTASXVCLLNNFL

SEQ ID NO: 53 - protein, 91 aa, Artificial Sequence; identical to SEQ ID NO: 37
  Description of Artificial Sequence Human mAb

SEQ ID NO: 54 - protein, 131 aa, Artificial Sequence
  Description of Artificial Sequence Human mAb
LGQPASISCRSSQSLVYSDGNTFLNWFQQRPGQSPRRLIYKVSNWDSGVPDRFSGSGSGTDFTLKISRVEAEDVGVYYCMQGTHWPITFGQGTRLEIKRTVAAPSVFIFPPSDEQLKSGTASVVCLLNNFY

SEQ ID NO: 55 - protein, 119 aa, Artificial Sequence
  Description of Artificial Sequence Human mAb
IRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPCSRSTSESTAALGCLVKDYFPEPVT

SEQ ID NO: 56 - protein, 98 aa, Artificial Sequence
  Description of Artificial Sequence Human mAb
SGGSVSSGDYYWTWIRQSPGKGLEWIGHIYYSGNTNRLYNPSLKSRLTISIDTSKTQFSLKLSSVTAADTAIYYCVRDRVTGAFDIWGQGTMVTVSSA

SEQ ID NO: 57 - protein, 139 aa, Artificial Sequence
  Description of Artificial Sequence Human mAb
GLVKPSQTLSLTCAISGDSVSSNSAAWNWIRQSPSRGLEWLGRTYYRSKWYNDSAVSVKSRITINPDTSKNQFSLQLNSVTPEDTAVYYCASWIAAAGFDSWGQGTLVTVSSGSASAPTLFPLVSCENSPSDTSSVAVG

SEQ ID NO: 58 - protein, 147 aa, Artificial Sequence
  Description of Artificial Sequence Human mAb
PGLVKPSQTLSLTCAISGDSVSSNSAAWNWIRQSPSRGLEWLGRTYYRSKWYNDYTVSVKSRITINPDTSKNQFSLQLNSVTPEDTAVYYCTRVGYSSGWGGYYYGMDVWGQGTTVTVSSGSASAPTLFPLVSCENSPSDTSSVAVG

SEQ ID NO: 59 - protein, 156 aa, Artificial Sequence
  Description of Artificial Sequence Human mAb
EVQLLESGGGLVQPXGSLRXSCAAXGFTFSSYAMTWVRQAPGKGLEWVSAISGSGGSTYYADSVKGRFTISRDNSENTLYLQMNSLRAEDTAVYYCANVSFQLWGQGTLVTVSSGSASAPTLFPLVSCENSPSDTSSVAVGCLAQDFLPDSITFYL

SEQ ID NO: 60 - protein, 147 aa, Artificial Sequence
  Description of Artificial Sequence Human mAb
DVQLLEXGGGLVQPXGXLRLSCAAXGFTFSSYAMXWVRQAXGKGLEWVSAISGSGGSTYYADSVKGRFTISRDNSKNTLYLQMNSLRAEDTAVYYCAKRWQWLVDNGYYYYYGMDVWGQGTTVTVSSGSASAPTLFPLVSCENSPSD

SEQ ID NO: 61 - protein, 152 aa, Artificial Sequence; identical to SEQ ID NO: 33
  Description of Artificial Sequence Human mAb

SEQ ID NO: 62 - protein, 149 aa, Artificial Sequence
  Description of Artificial Sequence Human mAb
QVQLVESGGGVVQPXRXLRXXCAASGFTFSSYGMHWVRQAXGKGLEWVAVIWYDGSNKYYADSVKGRFTISRDNSKNTLYLQMNSLRAEDTAVYYCARSPADWYFDLWGRGTLVTVSSASTKGPSVFPLAPCSRSTSESTAALGCLVKD

SEQ ID NO: 63 - protein, 157 aa, Artificial Sequence; identical to SEQ ID NO: 34
  Description of Artificial Sequence Human mAb

SEQ ID NO: 64 - protein, 160 aa, Artificial Sequence
  Description of Artificial Sequence Human mAb
EVQVVESGGGLIQPGGSLRLSCAASGFTVSRNYMNWVRQAPGKGLEWVSVIYSGGNTYYADSVKGRFTISRDNSKNTLYLQMNSLRAEDTAVYYCATGXSGYDYYYYGMDVWGQGTTVTVSSASTKGPSVFPLAPCSRSTSESTAALGCLVKDXXPEPVT